(19) United States
(12) Patent Application Publication
(10) Pub. No.: US 2023/0389497 A1
(43) Pub. Date: Dec. 7, 2023

(54) METHODS FOR CEREAL CROP HYBRID TEST CROSS EVALUATION

(71) Applicant: KWS SAAT SE & Co. KGaA, Einbeck (DE)

(72) Inventors: Jacob LAGE, Thriplow (GB); Carus JOHN-BEJAI, Thriplow (GB); Nicholas BIRD, Thriplow (GB)

(73) Assignee: KWS SAAT SE & Co. KGaA, Einbeck (DE)

(21) Appl. No.: 18/034,286

(22) PCT Filed: Oct. 28, 2021
(86) PCT No.: PCT/EP2021/079942

§ 371 Date: Apr. 27, 2023

(30) Foreign Application Priority Data
Oct. 28, 2020 (EP) 20204432.7

(51) Int. Cl. A01H 1/02; A01H 6/46

(52) U.S. Cl. CPC A01H 1/023; A01H 6/4678

(57) ABSTRACT

The present invention relates to plant hybrid cross testing, such as in cereal crops such as Triticum, preferably Triticum aestivum. The invention in particular relates to methods for evaluating test crosses, in particular for evaluating heterosis and/or (general and/or specific) combining ability by using male sterile plants, which allows the use of small scale planting schemes comprising a limited number of parallel rows of tester and breeding lines.

### FIELD OF THE INVENTION

The invention relates to methods for generating and using genetically (male) sterile hybrid plant testers, in particular plants of the genus Triticum. Furthermore, the present invention provides a method for facilitating production of initial test crossing in hybrid test crosses with representative testers from male and female pools.

### BACKGROUND OF THE INVENTION

In hybrid crop breeding, crossing different inbred lines typically results in F1 hybrids that have higher yields than their respective parents. This phenomenon where the hybrid outperforms its parents is known as heterosis. Hybrid crop breeding and in particular the production of sufficient amounts of hybrid seeds in self-pollinating crops such as wheat is very challenging. To combat this issue, the use of chemical hybridizing agents (CHA) has been implemented for instance in wheat hybrid production. This method not only enables the production of hybrid seeds of any parental combination, but is also more convenient for promoting heterosis as no maintainer line or pre-breeding is required.

In hybrid breeding, all lines developed in line breeding can serve as potential parents, rendering the number of factorial crosses rapidly prohibitive. Therefore, lines are usually tested for their general combining ability (GCA) using a tester from the opposite heterotic group.

Currently in hybrid wheat breeding all test crosses for general combining ability (GCA) analysis are made by using chemical hybridization agents (CHA). The term CHA describes this class of chemicals in hybrid seed production that cause male sterility and, depending on mode of action and dosage, can sometimes lead to female sterility (McRae, 1985, Plant Breeding Reviews, Vol. 3, Chapter 3 “Advances in Chemical Hybridization). An advantage inherent to CHA use is that male sterility can be induced in the female inbred parent by simply spraying a chemical, therefore significantly reducing production costs. A CHA is only useful for commercial hybrid seed production if it selectively induces male and not female sterility, is genotype independent, and has systemic activity and persistence to allow for different stages of maturity among the treated plants (Whitford et al. Journal of Experimental Botany, 2013, Vol. 64, No. 18, pp 5411-5428).

However, this method is costly. Although the modern CHAs are effective across a broad range of genotypes and have reduced phytotoxicity, their commercial deployment is still hindered by a narrow window for application, which is subject to the prevailing environmental conditions. Furthermore, many lines do not become completely sterile with CHA and thus do not produce uniform hybrids. Given that the plots need to be sprayed with CHA in field conditions, relatively large plots need to be used.

It is an objective of the present invention to address one or more of the above shortcomings.

### SUMMARY OF THE INVENTION

The present invention relates to plant hybrid cross testing, such as in cereal crops such as Triticum, preferably Triticum aestivum. The invention in particular relates to methods for evaluating test crosses, in particular for evaluating heterosis and/or (general and/or specific) combining ability.

By converting testers from both the male and female pools to for instance the blue aleurone (Bla) hybrid system the inventors have realized that it is possible to generate seed of the testers which will grow into male sterile plants. As no CHA application is needed, the test cross production can be done in small plots saving land but also allowing for testcross production with very little seed. This furthermore allows for testcross production earlier in the breeding process where limited seed is available. In addition, male sterile testers from the male pool can still be used for production of test cross seed with lines relevant for the female pool where pollination ability is not a requirement. The fact that the tester and line can be grow in rows spaced <20 cm apart ensures sufficient seed set even from poor pollinators.

The present invention is in particular captured by any one or any combination of one or more of the below numbered statements 1 to 43, with any other statement and/or embodiments.
1. A method for generating a hybrid plant, preferably a hybrid cereal plant, more preferably a hybrid plant from the genus Triticum, most preferably a hybrid Triticum aestivum plant, comprising
crossing one or more first plant or plant population, line, cultivar, or variety with one or more second plant or plant population, line, cultivar or variety, wherein said first or second plant or plant population, line, cultivar, or variety is a (genetically) male sterile plant or plant population, line, cultivar, or variety.
2. A method for evaluating (general and/or specific) combining ability, comprising crossing one or more first plant or plant population, line, cultivar, or variety with one or more second plant or plant population, line, cultivar, or variety, wherein said first or second plant or plant population, line, cultivar, or variety is a (genetically) male sterile plant or plant population, line, cultivar, or variety, preferably wherein said first and second plant or plant population, line, cultivar, or variety is a cereal plant or plant population, line, cultivar, or variety, more preferably a plant or plant population, line, cultivar, or variety from the species Triticum, most preferably a Triticum aestivum plant or plant population, line, cultivar, or variety.
3. The method according to statement 2, further comprising analysing (general and/or specific) combining ability in (F1) progeny.
4. The method according to statement 3, wherein said (general and/or specific) combining ability is analysed for seed yield, biomass yield, plant height, days to anthesis.
5. A method for evaluating heterosis, comprising crossing one or more first plant or plant population, line, cultivar, or variety with one or more second plant or plant population, line, cultivar, or variety, wherein said first or second plant or plant population, line, cultivar, or variety is a (genetically) male sterile plant line, preferably wherein said first and second plant or plant population, line, cultivar, or variety is a cereal plant or plant population, line, cultivar, or variety, more preferably a plant or plant population, line, cultivar, or variety from the species Triticum, most preferably a Triticum aestivum plant or plant population, line, cultivar, or variety.
6. The method according to statement 5, further comprising analysing heterosis in (F1) progeny.
7. A method for plant hybrid test crossing, comprising crossing one or more first plant or plant population, line, cultivar, or variety tester with one or more second plant or plant population, line, cultivar, or variety selected from a or line, cultivar, or variety pool, wherein said first plant or plant population, line, cultivar, or variety is a (genetically) male sterile plant or plant population, line, cultivar, or variety, preferably wherein said first and second plant or plant population, line, cultivar, or variety is a cereal plant or plant population, line, cultivar, or variety, more preferably a plant or plant population, line, cultivar, or variety from the species Triticum, most preferably a Triticum aestivum plant or plant population, line, cultivar, or variety.
8. The method according to any of statements 1 to 7, comprising sowing seeds of said one or more first plant or plant population, line, cultivar, or variety or planting plants of said one or more first plant or plant population, line, cultivar, or variety in one or more parallel row;
sowing seeds of said one or more second plant or plant population, line, cultivar, or variety or planting plants of said one or more second plant or plant population, line, cultivar, or variety in one or more parallel row flanking or flanked by said one or more parallel row of said one or more first plant or plant population, line, cultivar, or variety.
9. A method for sowing or planting, comprising sowing seeds of one or more first plant or plant population, line, cultivar, or variety or planting plants of one or more first plant or plant population, line, cultivar, or variety in one or more parallel row;
sowing seeds of one or more second plant or plant population, line, cultivar, or variety or planting plants of one or more second plant or plant population, line, cultivar, or variety in one or more parallel row flanking or flanked by said one or more parallel row of said one or more first plant or plant population, line, cultivar, or variety;
wherein said first or second plant or plant population, line, cultivar, or variety is a (genetically) male sterile plant or plant population, line, cultivar, or variety, preferably wherein said first and second plant or plant population, line, cultivar, or variety is a cereal plant or plant population, line, cultivar, or variety, more preferably a plant or plant population, line, cultivar, or variety from the species Triticum, most preferably a Triticum aestivum plant or plant population, line, cultivar, or variety.
10. A method for sowing or planting for generating a hybrid plant, evaluating heterosis or general/specific combining ability, comprising sowing seeds of one or more first plant or plant population, line, cultivar, or variety or planting plants of one or more first plant or plant population, line, cultivar, or variety in one or more parallel row;
sowing seeds of one or more second plant or plant population, line, cultivar, or variety or planting plants of one or more second plant or plant population, line, cultivar, or variety in one or more parallel row flanking or flanked by said one or more parallel row of said one or more first plant or plant population, line, cultivar, or variety;
wherein said first or second plant or plant population, line, cultivar, or variety is a (genetically) male sterile plant or plant population, line, cultivar, or variety, preferably wherein said first and second plant or plant population, line, cultivar, or variety is a cereal plant or plant population, line, cultivar, or variety, more preferably a plant or plant population, line, cultivar, or variety from the species Triticum, most preferably a Triticum aestivum plant or plant population, line, cultivar, or variety.
11. A method for sowing or planting for plant hybrid test crossing, comprising sowing seeds of one or more first plant or plant population, line, cultivar, or variety or planting plants of one or more first plant or plant population, line, cultivar, or variety in one or more parallel row;
sowing seeds of one or more second plant or plant population, line, cultivar, or variety or planting plants of one or more second plant or plant population, line, cultivar, or variety in one or more parallel row flanking or flanked by said one or more parallel row of said one or more first plant or plant population, line, cultivar, or variety;
wherein said first or second plant or plant population, line, cultivar, or variety is a (genetically) male sterile plant or plant population, line, cultivar, or variety, preferably wherein said first and second plant or plant population, line, cultivar, or variety is a cereal plant or plant population, line, cultivar, or variety, more preferably a plant or plant population, line, cultivar, or variety from the species Triticum, most preferably a Triticum aestivum plant or plant population, line, cultivar, or variety.
12. The method according to any of statements 8 to 11, wherein said one or more row of said one or more first plant or plant population, line, cultivar, or variety is (at most) 5 rows, preferably (at most) 4 rows, more preferably (at most) three rows, most preferably (at most) two rows.
13. The method according to any of statements 8 to 12, wherein said one or more row of said one or more second plant or plant population, line, cultivar, or variety is (at most) 5 rows, preferably (at most) 4 rows, more preferably (at most) three rows, most preferably (at most) two rows.
14. The method according to any of statements 8 to 13, wherein each of said rows is (at most) 1 meter spaced apart.
15. The method according to any of statements 8 to 14, wherein each of said rows is (at most) 15 m long.
16. The method according to any of statements 8 to 15, wherein individual plants within a row are spaced apart 1 to 50 cm.
17. The method according to any of statements 8 to 16, wherein the plant density of said first and/or second plant or plant population, line, cultivar, or variety is from 10 to 500 plants/m².
18. The method according to any of statements 1 to 17, wherein the number of individual plants of said one or more first and/or second plant or plant population, line, cultivar, or variety is from 10 to 1000.
19. A method for evaluating plant hybrid test crosses or for evaluating (general and/or specific) combining ability or heterosis (in plant hybrid test crosses) of or in a particular plant or plant population, line, cultivar, or variety (or combination of parent plants) comprising
   providing a (F1) hybrid plant or plant population obtained by crossing said particular plant as a first or plant population, line, cultivar, or variety with a different plant or plant population, line, cultivar, or variety as a second plant or plant population, line, cultivar, or variety, wherein said first or second plant is a (genetically) male sterile plant (and wherein said other plant is a (genetically) male fertile plant);
   determining one or more (agronomic, physiologic, or quality) characteristics or traits of said hybrid plant or plant population (so as to evaluate the plant hybrid test cross or to determine the (general and/or specific) combining ability or heterosis of said particular plant or combination of parent plants),
preferably wherein said first and second plant is a cereal plant line, more preferably a plant from the genus Triticum, most preferably a Triticum aestivum plant.
20. The method according to any of statements 1 to 19, wherein said first plant or plant population, line, cultivar, or variety is a (genetically) male sterile plant or plant population, line, cultivar, or variety.
21. The method according to any of statements 1 to 20, wherein said second plant or plant population, line, cultivar, or variety is a (genetically) male sterile plant or plant population, line, cultivar, or variety.
22. The method according to any of statements 1 to 21, wherein said first and/or said second plant or plant population, line, cultivar, or variety is an inbred plant or plant population, line, cultivar, or variety.
23. The method according to any of statements 1 to 22, wherein said first or second plant or plant population, line, cultivar, or variety is a tester.
24. The method according to any of statements 1 to 23, wherein said first or second plant or plant population, line, cultivar, or variety is selected from plant or plant populations, lines, cultivars, or varieties in a female pool of plants or plant populations, lines, cultivars, or varieties.
25. The method according to statement 24, wherein said female pool of plants or plant populations, lines, cultivars, or varieties is characterised by its appropriateness and use as a female in hybrid breeding.
26. The method according to any of statements 1 to 25, wherein said first or second plant or plant population, line, cultivar, or variety is selected from a male pool of plants or plant populations, lines, cultivars, or varieties.
27. The method according to statement 26, wherein said male pool of plants or plant populations, lines, cultivars, or varieties is characterised by its appropriateness and use as a male in hybrid breeding.
28. The method according to any of statements 1 to 27, wherein said first plant or plant population, line, cultivar, or variety is a (genetically) male sterile plant or plant population, line, cultivar, or variety selected from plants or plant populations, lines, cultivars, or varieties in a female pool of plants or plant populations, lines, cultivars, or varieties.
29. The method according to any of statements 1 to 27, wherein said first plant or plant population, line, cultivar, or variety is a (genetically) male sterile plant or plant population, line, cultivar, or variety selected from plants or plant populations, lines, cultivars, or varieties in a male pool of plants or plant populations, lines, cultivars, or varieties.
30. The method according to any of statements 1 to 27, wherein said second plant or plant population, line, cultivar, or variety is a (genetically) male sterile plant or plant population, line, cultivar, or variety selected from plants or plant populations, lines, cultivars, or varieties in a female pool of plants or plant populations, lines, cultivars, or varieties.
31. The method according to any of statements 1 to 27, wherein said second plant or plant population, line, cultivar, or variety is a (genetically) male sterile plant or plant population, line, cultivar, or variety selected from plants or plant populations, lines, cultivars, or varieties in a male pool of plants or plant populations, lines, cultivars, or varieties.
32. The method according to any of statements 1 to 31, wherein said first and second plant or plant population, line, cultivar, or variety is a cereal.
33. The method according to any of statements 1 to 32, wherein said first and second plant or plant population, line, cultivar, or variety is from the family of Poaceae, preferably the subfamily Pooideae.
34. The method according to any of statements 1 to 33, wherein said first and second plant or plant population, line, cultivar, or variety is from the tribe Triticeae.
35. The method according to any of statements 1 to 34, wherein said first and second plant or plant population, line, cultivar, or variety are from the genus Triticum.
36. The method according to any of statements 1 to 35, wherein said first and said second plant or plant population, line, cultivar, or variety are from the species Triticum aestivum.
37. The method according to any of statements 1 to 36, wherein said method does not involve the use of a chemical hybridization agent and/or the use of cytoplasmic male sterility.
38. The method according to any of statements 1 to 37, wherein said (genetically) male sterile plant or plant population, line, cultivar, or variety comprises a homozygous mutation resulting in (genetical) male sterility.
39. The method according to any of statements 1 to 38, wherein said (genetically) male sterile plant or plant population, line, cultivar, or variety comprises a recessive mutation resulting in (genetical) male sterility.
40. The method according to any of statements 1 to 39, wherein said (genetically) male sterile plant or plant population, line, cultivar, or variety comprises a mutation is the ms1 and/or ms5 gene, preferably ms1, preferably a knockout mutation or a frameshift mutation.
41. The method according to any of statements 1 to 40, wherein said (genetically) male sterile plant or plant population, line, cultivar, or variety comprises the BLue Aleurone (BLA) system.
42. The method according to any of statements 1 to 41, wherein said first plant or plant population, line, cultivar, or variety comprises a sterility restorer gene.
43. The method according to any of statements 1 to 42, wherein said first plant or plant population, line, cultivar, or variety comprises a genetic sterility restorer gene.
44. The method according to any of statements 1 to 43, wherein said first plant or plant population, line, cultivar, or variety comprises a male sterility restorer gene.
45. The method according to any of statements 1 to 44, wherein said first plant or plant population, line, cultivar, or variety comprises a genetic male sterility restorer gene.
46. The method according to any of statements 42 to 45, wherein said first plant or plant population, line, cultivar, or variety comprises a selection marker.
47. The method according to any of statements 42 to 46, wherein said first plant or plant population, line, cultivar, or variety comprises a BLA gene or BLA gene coding sequence.
48. The method according to any of statements 46 to 47, wherein said selection marker or BLA gene and said restorer gene are linked.
49. The method according to any of statements 46 to 48, wherein said selection marker or BLA gene and said restorer gene reside on the same chromosome.
50. The method according to any of statements 46 to 49, wherein said selection marker or BLA gene and said restorer gene reside on the same chromosomal arm.
51. The method according to any of statements 42 to 50, wherein said restorer gene resides on an (alien) addition chromosome.
52. The method according to any of statements 1 to 41, wherein said second plant or plant population, line, cultivar, or variety comprises a sterility restorer gene.
53. The method according to any of statements 1 to 41 or 52, wherein said second plant or plant population, line, cultivar, or variety comprises a genetic sterility restorer gene.
54. The method according to any of statements 1 to 41 or 52 to 53, wherein said second plant or plant population, line, cultivar, or variety comprises a male sterility restorer gene.
55. The method according to any of statements 1 to 41 or 52 to 54, wherein said second plant or plant population, line, cultivar, or variety comprises a genetic male sterility restorer gene.
56. The method according to any of statements 52 to 55, wherein said second plant or plant population, line, cultivar, or variety comprises a selection marker.
57. The method according to any of statements 52 to 56, wherein said second plant or plant population, line, cultivar, or variety comprises a BLA gene or BLA gene coding sequence.
58. The method according to any of statements 56 to 57, wherein said selection marker or BLA gene and said restorer gene are linked.
59. The method according to any of statements 56 to 58, wherein said selection marker or BLA gene and said restorer gene reside on the same chromosome.
60. The method according to any of statements 56 to 59, wherein said selection marker or BLA gene and said restorer gene reside on the same chromosomal arm.
61. The method according to any of statements 52 to 60, wherein said restorer gene resides on an (alien) addition chromosome.
62. The method according to any of statements 1 to 41, wherein said first plant or plant population, line, cultivar, or variety is a (genetically) (male) sterile plant or plant population, line, cultivar, or variety obtained by or is obtainable by crossing a plant or plant population, line, cultivar, or variety comprising a (genetic) (male) sterility restorer gene, preferably comprising a (homozygous) ms (such as ms1) mutation (e.g. deletion or knockout), with a (genetic) (male) sterile plant.
63. The method according to any of statements 1 to 41, wherein said first plant or plant population, line, cultivar, or variety is a (genetically) (male) sterile plant or plant population, line, cultivar, or variety obtained by or is obtainable by crossing a plant or plant population, line, cultivar, or variety comprising a (genetic) (male) sterility restorer gene, preferably comprising a (homozygous) ms (such as ms1) mutation (e.g. deletion or knockout) and a selection marker, with a (genetic) (male) sterile plant.
64. The method according to any of statements 1 to 41, wherein said first plant or plant population, line, cultivar, or variety is a (genetically) (male) sterile plant or plant population, line, cultivar, or variety obtained by or is obtainable by crossing a plant or plant population, line, cultivar, or variety comprising a (genetic) (male) sterility restorer gene, preferably comprising a (homozygous) ms (such as ms1) mutation (e.g. deletion or knockout) and a BLA gene or BLA gene coding sequence, with a (genetic) (male) sterile plant.
65. The method according to any of statements 63 to 64, wherein said selection marker or BLA gene and said restorer gene are linked.
66. The method according to any of statements 63 to 65, wherein said selection marker or BLA gene and said restorer gene reside on the same chromosome.
67. The method according to any of statements 63 to 66, wherein said selection marker or BLA gene and said restorer gene reside on the same chromosomal arm.
68. The method according to any of statements 63 to 67, wherein said restorer gene resides on an (alien) addition chromosome.
69. The method according to any of statements 1 to 41, wherein said second plant or plant population, line, cultivar, or variety is a (genetically) (male) sterile plant or plant population, line, cultivar, or variety obtained by or is obtainable by crossing a plant or plant population, line, cultivar, or variety comprising a (genetic) (male) sterility restorer gene, preferably comprising a (homozygous) ms (such as ms1) mutation (e.g. deletion or knockout), with a (genetic) (male) sterile plant.
70. The method according to any of statements 1 to 41, wherein said second plant or plant population, line, cultivar, or variety is a (genetically) (male) sterile plant or plant population, line, cultivar, or variety obtained by or is obtainable by crossing a plant or plant population, line, cultivar, or variety comprising a (genetic) (male) sterility restorer gene, preferably comprising a (homozygous) ms (such as ms1) mutation (e.g. deletion or knockout) and a selection marker, with a (genetic) (male) sterile plant.
71. The method according to any of statements 1 to 41, wherein said second plant or plant population, line, cultivar, or variety is a (genetically) (male) sterile plant or plant population, line, cultivar, or variety obtained by or is obtainable by crossing a plant or plant population, line, cultivar, or variety comprising a (genetic) (male) sterility restorer gene, preferably comprising a (homozygous) ms (such as ms1) mutation (e.g. deletion or knockout) and a BLA gene or BLA gene coding sequence, with a (genetic) (male) sterile plant.
72. The method according to any of statements 70 to 71, wherein said selection marker or BLA gene and said restorer gene are linked.
73. The method according to any of statements 70 to 72, wherein said selection marker or BLA gene and said restorer gene reside on the same chromosome.
74. The method according to any of statements 70 to 73, wherein said selection marker or BLA gene and said restorer gene reside on the same chromosomal arm.
75. The method according to any of statements 70 to 74, wherein said restorer gene resides on an (alien) addition chromosome.
76. The method according to any of statements 1 to 41, wherein said first plant or plant population, line, cultivar, or variety is a (genetically) (male) sterile plant or plant population, line, cultivar, or variety obtained by or is obtainable by self-fertilizing a plant or plant population, line, cultivar, or variety comprising a (genetic) (male) sterility restorer gene, preferably comprising a (homozygous) ms (such as ms1) mutation (e.g. deletion or knockout).
77. The method according to any of statements 1 to 41, wherein said first plant or plant population, line, cultivar, or variety is a (genetically) (male) sterile plant or plant population, line, cultivar, or variety obtained by or is obtainable by self-fertilizing a plant or plant population, line, cultivar, or variety comprising a (genetic) (male) sterility restorer gene, preferably comprising a (homozygous) ms (such as ms1) mutation (e.g. deletion or knockout) and a selection marker.
78. The method according to any of statements 1 to 41, wherein said first plant or plant population, line, cultivar, or variety is a (genetically) (male) sterile plant or plant population, line, cultivar, or variety obtained by or is obtainable by self-fertilizing a plant or plant population, line, cultivar, or variety comprising a (genetic) (male) sterility restorer gene, preferably comprising a (homozygous) ms (such as ms1) mutation (e.g. deletion or knockout) and a BLA gene or BLA gene coding sequence.
79. The method according to any of statements 63 to 64, wherein said selection marker or BLA gene and said restorer gene are linked.
80. The method according to any of statements 63 to 65, wherein said selection marker or BLA gene and said restorer gene reside on the same chromosome.
81. The method according to any of statements 63 to 66, wherein said selection marker or BLA gene and said restorer gene reside on the same chromosomal arm.
82. The method according to any of statements 63 to 67, wherein said restorer gene resides on an (alien) addition chromosome.
83. The method according to any of statements 1 to 41, wherein said second plant or plant population, line, cultivar, or variety is a (genetically) (male) sterile plant or plant population, line, cultivar, or variety obtained by or is obtainable by self-fertilizing a plant or plant population, line, cultivar, or variety comprising a (genetic) (male) sterility restorer gene, preferably comprising a (homozygous) ms (such as ms1) mutation (e.g. deletion or knockout).
84. The method according to any of statements 1 to 41, wherein said second plant or plant population, line, cultivar, or variety is a (genetically) (male) sterile plant or plant population, line, cultivar, or variety obtained by or is obtainable by self-fertilizing a plant or plant population, line, cultivar, or variety comprising a (genetic) (male) sterility restorer gene, preferably comprising a (homozygous) ms (such as ms1) mutation (e.g. deletion or knockout) and a selection marker.
85. The method according to any of statements 1 to 41, wherein said second plant or plant population, line, cultivar, or variety is a (genetically) (male) sterile plant or plant population, line, cultivar, or variety obtained by or is obtainable by self-fertilizing a plant or plant population, line, cultivar, or variety comprising a (genetic) (male) sterility restorer gene, preferably comprising a (homozygous) ms (such as ms1) mutation (e.g. deletion or knockout) and a BLA gene or BLA gene coding sequence.
86. The method according to any of statements 70 to 71, wherein said selection marker or BLA gene and said restorer gene are linked.
87. The method according to any of statements 70 to 72, wherein said selection marker or BLA gene and said restorer gene reside on the same chromosome.
88. The method according to any of statements 70 to 73, wherein said selection marker or BLA gene and said restorer gene reside on the same chromosomal arm.
89. The method according to any of statements 70 to 74, wherein said restorer gene resides on an (alien) addition chromosome.
90. The method according to any of statements 1 to 41, wherein said first or second plant is obtained or obtainable by selecting white or non-blue seed derived from (a mix of seed collected from) a self-fertilized ms-deletion plant, preferably a ms1-deletion plant, having an (alien) addition chromosome comprising a restorer gene and a selection marker, preferably a BLA gene of BLA gene coding sequence.
91. The method according to any of statements 1 to 90, comprising
(a) crossing a plant or plant population, line, cultivar, or variety comprising a (genetic) (male) sterility restorer gene, preferably comprising a (homozygous) ms (such as ms1) mutation (e.g. deletion or knockout), with a (genetic) (male) sterile plant;
(b) selecting (genetic) (male) sterile offspring, e.g. seed (not containing the restorer gene);
(c) crossing said offspring as a first plant or plant population, line, cultivar, or variety with one or more second plant or plant population, line, cultivar or variety (which is a (genetically) (male) fertile plant or plant population, line, cultivar, or variety) to generate a hybrid plant (or part thereof, e.g. seed) or plant population;
(d) optionally further determining one or more (agronomic, physiologic, or quality) characteristics or traits of said hybrid plant or plant population (so as to evaluate the plant hybrid test cross or to determine the (general and/or specific) combining ability or heterosis of said particular plant or combination of parent plants).
92. The method according to any of statements 1 to 90, comprising
(a) self-fertilizing a plant or plant population, line, cultivar, or variety comprising a (genetic) (male) sterility restorer gene, preferably comprising a (homozygous) ms (such as ms1) mutation (e.g. deletion or knockout);
(b) selecting (genetic) (male) sterile offspring, e.g. seed (not containing the restorer gene);
(c) crossing said offspring as a first plant or plant population, line, cultivar, or variety with one or more second plant or plant population, line, cultivar or variety (which is a (genetically) (male) fertile plant or plant population, line, cultivar, or variety) to generate a hybrid plant (or part thereof, e.g. seed) or plant population;
(d) optionally further determining one or more (agronomic, physiologic, or quality) characteristics or traits of said hybrid plant or plant population (so as to evaluate the plant hybrid test cross or to determine the (general and/or specific) combining ability or heterosis of said particular plant or combination of parent plants).
93. The method according to any of statements 91 or 92, wherein said plant or plant population, line, cultivar, or variety of step (a) comprising a (genetic) (male) sterility restorer gene further comprises a selection marker.
94. The method according to any of statements 91 to 93, wherein said plant or plant population, line, cultivar, or variety of step (a) comprising a (genetic) (male) sterility restorer gene further comprises a BLA gene or BLA gene coding sequence.
95. The method according to any of statements 93 to 94, wherein said selection marker or BLA gene and said restorer gene are linked.
96. The method according to any of statements 93 to 95, wherein said selection marker or BLA gene and said restorer gene reside on the same chromosome.
97. The method according to any of statements 93 to 96, wherein said selection marker or BLA gene and said restorer gene reside on the same chromosomal arm.
98. The method according to any of statements 93 to 97, wherein said restorer gene resides on an (alien) addition chromosome.
99. The method according to any of statements 93 to 98, wherein said selection in step (b) is based on said selection marker.
100. The method according to any of statements 94 to 99, wherein selecting in step (b) comprises selecting non-blue seed derived from a mix of seed collected from the cross or self-feralization of step (a).
101. Use of a (genetically) male sterile plant or plant population, line, cultivar, or variety for generating a hybrid plant, evaluating heterosis or general/specific combining ability, or for plant hybrid test crossing, preferably wherein said plant or plant population, line, cultivar, or variety is as defined in any of statements 1 to 100, preferably wherein said plant or plant population, line, cultivar, or variety is a cereal plant line, more preferably a plant or plant population, line, cultivar, or variety from the species Triticum, most preferably a Triticum aestivum plant or plant population, line, cultivar, or variety.
102. The method or use according to any of the previous statements, wherein said plant, plant line, first plant, first plant line, second plant, and/or second plant line is transgenic or mutagenized.

### BRIEF DESCRIPTION OF THE FIGURES

FIG. 1: A. Schematic representation of conventional use of a female tester which is treated with CHA. This can then be crossed with lines already in the male pool or “exotic” lines which are being evaluated for the usefulness in the male pool; B. Schematic representation of conventional use of a male tester which is used to pollinate lines already in the female pool or “exotic” lines which are being evaluated for the usefulness in the female pool. For hybrid seed to be produced, all female lines need to be treated with CHA; C. Schematic representation of an embodiment of the invention, where the female tester is a Bla line, and non-blue seed are selected and grow into sterile (ms1 ms1) plants. This can then be crossed with lines already in the male pool or “exotic” lines which are being evaluated for the usefulness in the male pool; D. Schematic representation of an embodiment of the invention, where the Bla system is incorporated into the male tester. Non-blue seed of this can be grown with any potential female line (exotic or already in pool) which does not contain the Bla system.

FIG. 2: A. Schematic representation of traditional setup using CHA and planting large plots to accommodate spraying; B. Schematic representation of an embodiment of the invention using genetically sterile tester drilling in rows saving seed, space and chemical.

FIG. 3: Representation of the seed set in kg harvested from 6 m of double rows of females in relation to anther extrusion of the male used.

FIG. 4: Representation of the seed set in kg harvested from 6 m of double rows of females in relation to difference in heading date between the male and female.

FIG. 5 Representation of the amount of harvested grain (in grams) in relation to the difference in heading time (in days) between female and male parents (negative=male earlier than female).

FIG. 6 Representation of the amount of harvested grain (in grams) in relation to the anther extrusion level for the male parent (higher scores are better than lower scores).

FIG. 7 shows the amount of harvested grain (in grams) in relation to the difference in plant height (in cm) between the female and male parent (negative=female shorter than male).

### DETAILED DESCRIPTION OF THE INVENTION

Before the present system and method of the invention are described, it is to be understood that this invention is not limited to particular systems and methods or combinations described, since such systems and methods and combinations may, of course, vary. It is also to be understood that the terminology used herein is not intended to be limiting, since the scope of the present invention will be limited only by the appended claims.

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. It will be appreciated that the terms “comprising”, “comprises” and “comprised of” as used herein comprise the terms “consisting of”, “consists” and “consists of”, as well as the terms “consisting essentially of”, “consists essentially” and “consists essentially of”.

The recitation of numerical ranges by endpoints includes all numbers and fractions subsumed within the respective ranges, as well as the recited endpoints.

The term “about” or “approximately” as used herein when referring to a measurable value such as a parameter, an amount, a temporal duration, and the like, is meant to encompass variations of +/−20% or less, preferably +/−10% or less, more preferably +/−5% or less, and still more preferably +/−1% or less of and from the specified value, insofar such variations are appropriate to perform in the disclosed invention. It is to be understood that the value to which the modifier “about” or “approximately” refers is itself also specifically, and preferably, disclosed.

Whereas the terms “one or more” or “at least one”, such as one or more or at least one member(s) of a group of members, is clear per se, by means of further exemplification, the term encompasses inter alia a reference to any one of said members, or to any two or more of said members, such as, e.g., any ≥3, ≤4, ≥5, ≥6 or ≥7 etc. of said members, and up to all said members.

All references cited in the present specification are hereby incorporated by reference in their entirety. In particular, the teachings of all references herein specifically referred to are incorporated by reference.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the present invention.

Standard reference works setting forth the general principles of recombinant DNA technology include Molecular Cloning: A Laboratory Manual, 2nd ed., vol. 1-3, ed. Sambrook et al., Cold Spring Harbor Laboratory Press, Cold Spring Harbor, N.Y., 1989; Current Protocols in Molecular Biology, ed. Ausubel et al., Greene Publishing and Wiley-Interscience, New York, 1992 (with periodic updates) (“Ausubel et al. 1992”); the series Methods in Enzymology (Academic Press, Inc.); Innis et al., PCR Protocols: A Guide to Methods and Applications, Academic Press: San Diego, 1990; PCR 2: A Practical Approach (M. J. MacPherson, B. D. Hames and G. R. Taylor eds. (1995); Harlow and Lane, eds. (1988) Antibodies, a Laboratory Manual; and Animal Cell Culture (R. I. Freshney, ed. (1987). General principles of microbiology are set forth, for example, in Davis, B. D. et al., Microbiology, 3rd edition, Harper & Row, publishers, Philadelphia, Pa. (1980).

In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art. For example, in the appended claims, any of the claimed embodiments can be used in any combination. In the following detailed description of the invention, reference is made to the accompanying drawings that form a part hereof, and in which are shown by way of illustration only of specific embodiments in which the invention may be practiced. It is to be understood that other embodiments may be utilised, and structural or logical changes may be made without departing from the scope of the present invention. The following detailed description, therefore, is not to be taken in a limiting sense, and the scope of the present invention is defined by the appended claims.

Preferred statements (features) and embodiments of this invention are set herein below. Each statements and embodiments of the invention so defined may be combined with any other statement and/or embodiments unless clearly indicated to the contrary. In particular, any feature indicated as being preferred or advantageous may be combined with any other feature or features or statements indicated as being preferred or advantageous.

As used herein, the term “plant” includes whole plants, including descendants or progeny thereof. As used herein unless clearly indicated otherwise, the term “plant” intends to mean a plant at any developmental stage. Preferably, the plant according to the present invention are (predominantly) self-pollinating, i.e. a significant portion of the seeds produced result from self-pollination and not cross-pollination. Cross-pollination, also called allogamy, occurs when pollen is delivered from the stamen of one flower to the stigma of a flower on another plant of the same species. Self-pollination, as opposed to cross-pollination refers to fertilization of ovules/female gametes in a plant by pollen from the same plant. Self-pollination occurs when pollen from one flower pollinates the same flower or other flowers of the same individual. Self-pollination may include autogamy, where pollen is transferred to the female part of the same flower; or geitonogamy, when pollen is transferred to another flower on the same plant. In certain embodiments, sel-pollination involves cleistogamy. Preferably at least 25% of the seeds produced result from self-pollination, more preferably at least 50%, even more preferably as at least 75%, most preferably at least 90%. The term “plant part” includes any part or derivative of the plant, including particular plant tissues or structures, plant cells, plant protoplast, plant cell or tissue culture from which plants can be regenerated, plant calli, plant clumps and plant cells that are intact in plants or parts of plants, such as seeds, kernels, cobs, flowers, cotyledons, leaves, stems, buds, roots, root tips, stover, and the like. Plant parts may include processed plant parts or derivatives, including flower, oils, extracts etc. “Parts of a plant” are e.g. shoot vegetative organs/structures, e.g., leaves, stems and tubers; roots, flowers and floral organs/structures, e.g. bracts, sepals, petals, stamens, carpels, anthers and ovules; seed, including embryo, endosperm, and seed coat; fruit and the mature ovary; plant tissue, e.g. vascular tissue, ground tissue, and the like; and cells, e.g. guard cells, egg cells, pollen, trichomes and the like; and progeny of the same. Parts of plants may be attached to or separate from a whole intact plant. Such parts of a plant include, but are not limited to, organs, tissues, and cells of a plant, and preferably seeds. A “plant cell” is a structural and physiological unit of a plant, comprising a protoplast and a cell wall. The plant cell may be in form of an isolated single cell or a cultured cell, or as a part of higher organized unit such as, for example, plant tissue, a plant organ, or a whole plant. “Plant cell culture” means cultures of plant units such as, for example, protoplasts, cell culture cells, cells in plant tissues, pollen, pollen tubes, ovules, embryo sacs, zygotes and embryos at various stages of development. “Plant material” refers to leaves, stems, roots, flowers or flower parts, fruits, pollen, egg cells, zygotes, seeds, cuttings, cell or tissue cultures, or any other part or product of a plant. This also includes callus or callus tissue as well as extracts (such as extracts from taproots) or samples. A “plant organ” is a distinct and visibly structured and differentiated part of a plant such as a root, stem, leaf, flower bud, or embryo. “Plant tissue” as used herein means a group of plant cells organized into a structural and functional unit. Any tissue of a plant in planta or in culture is included. This term includes, but is not limited to, whole plants, plant organs, plant seeds, tissue culture and any groups of plant cells organized into structural and/or functional units. The use of this term in conjunction with, or in the absence of, any specific type of plant tissue as listed above or otherwise embraced by this definition is not intended to be exclusive of any other type of plant tissue.

The invention may be applied to plant parts or derivatives. In certain embodiments, the plant part or derivative is or comprises (functional) propagation material, such as germplasm, a seed, or plant embryo or other material from which a plant can be regenerated. In certain embodiments, the plant part or derivative is not (functional) propagation material, such as germplasm, a seed, or plant embryo or other material from which a plant can be regenerated. In certain embodiments, the plant part or derivative does not comprise (functional) male and female reproductive organs. In certain embodiments, the plant part or derivative is or comprises propagation material, but propagation material which does not or cannot be used (anymore) to produce or generate new plants, such as propagation material which have been chemically, mechanically or otherwise rendered non-functional, for instance by heat treatment, acid treatment, compaction, crushing, chopping, etc.

As used herein, the terms “progeny” and “progeny plant” refer to a plant generated from sexual reproduction from one or more parent plants. A progeny plant can be obtained by selfing a single parent plant, or by crossing two parental plants. For instance, a progeny plant can be obtained by selfing of a parent plant or by crossing two parental plants and include selfings as well as the F1 or F2 or still further generations. An F1 is a first-generation progeny produced from parents at least one of which is used for the first time as donor of a trait, while progeny of second generation (F2) or subsequent generations (F3, F4, and the like) are specimens produced from selfings, intercrosses, backcrosses, and/or other crosses of F1 s, F2 s, and the like. An F1 can thus be (and in some embodiments is) a hybrid resulting from a cross between two true breeding parents (i.e., parents that are true-breeding are each homozygous for a trait of interest or an allele thereof), while an F2 can be (and in some embodiments is) a progeny resulting from self-pollination of the F1 hybrids. The term “progeny” can in certain embodiments be used interchangeably with “offspring”, in particular when the plant or plant material is derived from sexual crossing of parent plants. According to the present invention, progeny preferably refers to the F1 progeny.

The invention described herein may be applied to a plant. In certain embodiments, the plant is a crop plant, such as a cash crop or subsistence crop, such as food or non-food crops, including agriculture, horticulture, floriculture, or industrial crops. The term crop plant has its ordinary meaning as known in the art. By means of further guidance, and without limitation, a crop plant is a plant grown by humans for food and other resources, and can be grown and harvested extensively for profit or subsistence, typically in an agricultural setting or context.

The term “cereal plant” as used herein refers to a crop plant of the grass family (i.e., Graminaceae or Poaceae) cultivated for the food value of their grains, such as, but not limited to, wheat, triticale, corn, rice, barley, oat, rye, sorghum, millet, buckwheat, fonio, and quino. In certain embodiments, the cereal plant is a tetraploid wheat, a hexaploid wheat, triticale, maize, rice, barley, or oats. In certain embodiments, the cereal plant is wheat (e.g., any species of the genus Triticum, including progenitors thereof, as well as progeny thereof produced by crosses with other species). In certain embodiments, the cereal plant is a tetraploid wheat or a hexaploid wheat. Hexaploid wheat (e.g., genome organization of AABBDD), comprised of 42 chromosomes, and includes, for example, T. aestivum, T. spelta, T. mocha, T. compaction, T. sphaerococcum, T. vavilovii, and interspecies cross thereof. Tetraploid wheat (e.g., genome organization of AABB), comprised of 28 chromosomes, and includes, for example, T. durum (also referred to as durum wheat or Triticum turgidum ssp. durum), T. dicoccoides, T. dicoccum, T polonicum, and interspecies cross thereof. Wheat can also include possible progenitors of hexaploid or tetraploid Triticum sp. such as T. uartu, T. monococcum or T. boeoticum for the A genome, Aegilops speltoides for the B genome, and T. tauschii (also known as Aegilops squarrosa or Aegilops tauschii) for the D genome. In certain embodiments, the cereal plant is a Triticum durum or Triticum aestivum.

As used herein, the term “Poaceae” refers to the family of grasses, or Gramineae. Preferably, the Poaceae are cereals (or cereal grasses), which are in particular cultivated for the edible components of its grain.

As used herein, the term “Pooideae” refers to the subfamily of Poaceae in the Poaceae family. Preferably, the Pooideae are cereals (or cereal grasses), which are in particular cultivated for the edible components of its grain.

As used herein, the term “Triticeae” refers to the tribe of Triticeae in the ppoideae subfamily.

Preferably, the Triticeae are cereals (or cereal grasses), which are in particular cultivated for the edible components of its grain. Non limiting genera in the tribe Triticaceae include Aegilops, Agropyron, Amblyopyrum, Anthosachne, Australopyrum, Cockaynea, Crithopsis, Dasypyrum, Elymus, Elytrigia, Eremium, Eremopyrum, Festucopsis, Haynaldia, Henrardia, Heteranthelium, Hordelymus, Hordeum, Hystrix, Kengyilia, Leymus, Lophopyrum, Malacurus, Pascopyrum, Peridictyon, Psathyrostachys, Pseudoroegneria, Secale, Sitanion, Stenostachys, Taeniatherum, Thinopyrum, Triticum. Preferably, the Triticeae genus is Triticum or Hordeum.

As used herein, the term “Triticum” refers to the genus Triticum in the Triticeae tribe. The term Triticum may be used herein interchangeably with wheat. Non limiting species in the genus Triticum include T. aestivum, T. aethiopicum, T. araraticum, T. boeoticum, T. carthlicum, T. compactum, T. dicoccoides, T. dicoccon, T. durum, T. ispahanicum, T. karamyschevii, T. macha, T. militinae, T. monococcum, T. polonicum, T. spelta, T. sphaerococcum, T. timopheevii, T. turanicum, T. turgidum, T. urartu, T. vavilovii, T. zhukovskyi; or any subspecies or hybrid thereof, including all ploidy levels, such as (allo)tetraploid and (allo)hexaploid. Preferably, the Triticum species is Triticum aestivum.

The present invention relates to methods for generating a hybrid plant comprising crossing one or more first plant or plant population, line, cultivar, or variety with one or more second plant or plant population, line, cultivar or variety, wherein said first or second plant or plant population, line, cultivar, or variety is a (genetically) male sterile plant or plant population, line, cultivar, or variety.

The term “hybrid”, “hybrid plant”, or hybrid seed” as used in the context of the present invention has its ordinary meaning known in the art. By means of further guidance, and without limitation in the context of the present invention this term refers to the offspring of two (genetically distinct or different) parent plants, which may be different plant lines, cultivars, or varieties. It will be understood that according to the present invention, the parents of a hybrid plant preferably are from the same genus, preferably the same species. Preferably, the parents of a hybrid each are stable populations, having a high degree of homozygosity. The parents typically differ from each other in one or more traits or (agronomic, physiologic, or quality) characteristics. The hybrid therefore typically is heterozygous for such trait or (agronomic, physiologic, or quality) characteristic. According to the present invention, hybrids preferably are the F1 hybrids, i.e. the first generation of offspring resulting from the two parents (e.g. the two parental lines, cultivars, or varieties). The seed produced by crossing two parents is therefore the F1 hybrid seed.

The terms “plant line”, “plant cultivar”, and “plant variety” as used herein have their ordinary meaning in the art, and may be used herein interchangeably, unless explicitly indicated otherwise. By means of further guidance, and without limitation, different plant lines, cultivars, or varieties typically can be distinguished from each other by one or more traits or (agronomic, physiologic, or quality) characteristics. Cultivar and variety are commonly used to describe a line selected in a breeding program for mass production by farmers, variety being the most common term. According to the present invention, the term “plant line” is preferred. Preferably according to the invention, the plant line is an inbred plant line. Inbred lines can be produced as is known in the art, for instance through successive rounds of backcrossing. Inbred lines typically have a high degree of homozygosity. In certain embodiments, the plants, plant parts, or plant population have a (average) degree of homozygosity of at least 50%, preferably at least 60%, more preferably at least 70%, most preferably at least 80%, such as at least 90%. Preferably, a plant line as used herein is completely or almost homozygous (preferably identical plants all with the same ancestry), preferably having a (average) degree of homozygosity of at least 50%, preferably at least 60%, more preferably at least 70%, most preferably at least 80%, such as at least 90%.

As used herein, the term “plant population” may be used interchangeably with population of plants. A plant population preferably comprises a multitude of individual plants (often or typically related to each other through common ancestry), such as preferably at least 10, such as 20, 30, 40, 50, 60, 70, 80, or 90, more preferably at least 100, such as 200, 300, 400, 500, 600, 700, 800, or 900, even more preferably at least 1000, such as at least 10000 or at least 100000. In certain embodiments, a plant population as used herein refers to a population of plants of a single plant species, line, cultivar, or variety. In certain embodiments, the plant population (or plant parts thereof) is a plant line, strain, or variety. In certain embodiments, the plant population (or plant parts thereof) is not a plant line, strain, or variety. In certain embodiments, the plant population (or plant parts thereof) is an inbred plant line, strain, or variety. In certain embodiments, the plant population (or plant parts thereof) is not an inbred plant line, strain, or variety. In certain embodiments, the plant population (or plant parts thereof) is an outbred plant line, strain, or variety. In certain embodiments, the plant population (or plant parts thereof) is not an outbred plant line, strain, or variety.

As used herein, the terms “crossed” or “cross” or “crossing” means the fusion of gametes via pollination to produce progeny (i.e., cells, seeds, or plants). The term encompasses both sexual crosses (the pollination of one plant by another) and self-fertilization (selfing, self-pollination, i.e., when the pollen and ovule (or microspores and megaspores) are from the same plant or genetically identical plants). Preferably, crossing as referred to herein fertilization of one plant by another plant, i.e. not self-pollination.

As used herein, the term “male sterile” plant (line, cultivar, or variety) has its ordinary meaning in the art. By means of further guidance, and without limitation, the term refers to a plant which is unable to produce offspring as a pollen donor, and may result from the failure to produce (functional) anthers, pollen, or gametes. Cytoplasmic male sterile plants have cytoplasmic genes, usually in the mitochondria, that encode factors that disrupt or prevent pollen development, making them male-sterile, with male sterility inherited maternally. The utilization of cytoplasmic male sterility for hybrid seed production requires three separate plant lines: the male-sterile line, an isogeneic male-fertile line for propagation (“maintainer line”) and a line for restoring fertility to the hybrid so that it can produce seed (“restorer line”). The male-sterile line is used as the receptive parent in a hybrid cross, the maintainer line is genetically identical to the male-sterile line, excepting that it lacks the cytoplasmic sterility factors, and the restorer line is any line that masks the cytoplasmic sterility factor. The restorer line is very important for those plants, such as grain sorghum or cotton, the useful crop of which is the seed itself or seed-associated structures. Genetic male sterility is similar to cytoplasmic male sterility, but differs in that the sterility factors are encoded in nuclear DNA. Typically, genetic male sterility refers to a change in a plant's genetic structure which results in its ability to produce and/or spread viable pollen. Genetic male sterile plant lines may occur naturally. It is also possible to create a male-sterile plant line using recombinant techniques. Whether naturally occurring or transgenic, male-sterile lines still require the use of a sister maintainer line for their propagation, which of necessity leads to a minimum of 50% male-fertile plants in propagated seed. This is a result of the genetics of male-sterility and maintainer lines. If the male-sterility factor is recessive, as most are, a male-sterile plant would have to be homozygous recessive in order to display the trait. Preferably, according to the invention male sterility refers to genetical male sterility. Preferably, according to the invention male sterility is not or does not encompass cytoplasmic male sterility.

Essential for any hybrid system (in particular in self-pollinating crops) is the production of male-sterile female parents. WO 92/01366 A1 from Pacific Seeds Pty. Ltd. discloses a (genetical) male sterility system which allows the maintenance of male sterility. Male sterility can be achieved in a plant by a homozygous deletion on the short arm of chromosome 4B, such as in wheat. The deletion typically used is the well-known ‘Probus’ deletion (Fossati A, Ingold M. 1970. A male sterile mutant in Triticum aestivum. Wheat Inform Serv 30:8-10). Recently, the ms1 gene located in the region concerned by the deletion has been identified as the causative gene. If this gene is deleted physically or knocked out/down by a mutation or targeted modification (e.g. WO 2016/048891 A1, which is incorporated herein in its entirety for all intended purposes) then a reliable male sterility can be established. Fertility can in that case also be easily restored when a wheat line carrying the deletion or the mutation/modification homozygously, is crossed with any normal wheat. Resulting progenies are fertile as the deletion or the mutation/modification is only heterozygously present. However, in order to maintain the male-sterile female parent further components are needed. As such, WO 92/01366 teaches the use of a male parent that is isogenic to the female but has an alien addition chromosome bearing a dominant male fertility restorer gene from Triticum boeticum (trivial name is Triticum thaoudar) on the short arm and the BLue Aleurone (BLA) gene from Agropyron elongatum on the long arm, in a cross with the female parent for maintenance of the male sterile female parent, whereby the BLA gene, if expressed, confers a characteristic blue coloration of the progeny seed. The restorer gene and BLA gene may equally reside on the same chromosomal arm, i.e. on the same side of the centromere of the chromosome (see for instance WO2019043082, incorporated herein in its entirety). Recent studies indicate that the alien addition chromosome may also bear chromatin of Triticum aestivum. For the harvest from that cross, which generates a population of progeny seeds consisting of a mixture of the two parents, it is possible to physically separate the progeny seeds on the basis of the color marker, whereby, in theory, white seeds are still male-sterile due to the presence of the defect in the ms1 gene (deletion or mutation/modification) and the absence of the alien addition chromosome. These white seeds can be used as female parents in subsequent hybrid wheat production. The harvested blue seeds can be used as male parents for maintenance breeding. A similar concept is applicable for the ms2, ms3, ms4, and ms5 gene. A method for producing cereal plant comprising a monosomic alien addition chromosome carrying a male fertility restorer gene and at least one selection marker gene has been disclosed in WO 2019/043082, incorporated herein by reference in its entirety.

The systems described in WO 92/01366 and WO 2019/043082 may be used for practicing the present invention. Preferably, in such case, the plant is a cereal, preferably from the genus Triticum, more preferably Triticum aestivum. Accordingly, in certain embodiments a first or second plant, or plant population, line, cultivar, or variety as described herein may be (genetically) male sterile and may comprise a mutation resulting in (genetic) male sterility. In certain embodiments a first or second plant, or plant population, line, cultivar, or variety as described herein may be (genetically) male sterile and may comprise a mutation in any one or more of the ms1, ms2, ms3, ms4, and ms5 gene (resulting in (genetic) male sterility), preferably ms1. In certain embodiments a first or second plant, or plant population, line, cultivar, or variety as described herein may be (genetically) male sterile and may comprise a mutation in the ms1 gene (resulting in (genetic) male sterility). In certain embodiments a first or second plant, or plant population, line, cultivar, or variety as described herein may be (genetically) male sterile and may comprise a mutation in the ms2 gene (resulting in (genetic) male sterility). In certain embodiments a first or second plant, or plant population, line, cultivar, or variety as described herein may be (genetically) male sterile and may comprise a mutation in the ms3 gene (resulting in (genetic) male sterility). In certain embodiments a first or second plant, or plant population, line, cultivar, or variety as described herein may be (genetically) male sterile and may comprise a mutation in the ms4 gene (resulting in (genetic) male sterility). In certain embodiments a first or second plant, or plant population, line, cultivar, or variety as described herein may be (genetically) male sterile and may comprise a mutation in the ms5 gene (resulting in (genetic) male sterility). Preferably, all alleles of ms(1) are mutated. The mutations may be the same or different. In certain embodiments, the mutation is homozygous. It will be understood that the term “mutation” in this context includes physical mutations, such as deletions (of parts of the gene or coding sequence), point mutations, insertions, knockout mutations, etc., as also described herein elsewhere, as well as for instance knockdown of the gene (e.g. knockdown of expression). As used herein, the “mutations” result in decreased (preferably at least 50% decreased, more preferably at least 75% decreased, most preferably at least 90%, 95%, or 99% decreased) or (substantially) absent protein and/or mRNA expression levels or activity compared to the wild type/native/unmutated protein.

As used herein, the terms “restorer”, “restorer gene”, and “(male) fertility restorer (gene)” may be used interchangeably. The term “restorer gene” as used herein also refers to the coding sequence of the gene. These terms refer to a gene or a chromosomal segment or locus comprising a gene which is capable of restoring fertility, in particular male fertility, in otherwise (male) sterile plants, in particular plants characterised by genetic or nuclear male sterility, such as having mutations in one or more ms gene (conferring (genetic/nuclear) male sterility, such as (recessive) mutations in the ms1 gene which cause (genetic/nuclear) male sterility. Such mutations in ms genes, including ms1 mutations, are known in the art, and include among others ms(1) gene deletion, ms(1) gene knockdown, or ms(1) gene knockout. In certain embodiments the mutation is homozygous. In certain embodiments, the restorer gene is an (dominant) unmutated/wild type/native ms gene (i.e. Ms), such as Ms1. In certain embodiments, the restorer gene has a sequence corresponding to SEQ ID NO: 1, 6, 7, 8, or 10 of WO2019043082, or fragments or variants thereof that produce functional amino acid sequences; (ii) a nucleic acid sequence with at least 80%, at least 85%, at least 90%, at least 95%, at least 96%), at least 97%, at least 98%, or at least 99% sequence identity to the nucleic acid sequence as set forth in SEQ ID NO: 1, 6, 7, 8, or 10 of WO2019043082, or fragments thereof that produce functional amino acid sequences; (iii) a nucleic acid sequence having a coding sequence as set forth in SEQ ID NO: 2, 4, 9, 11, or 14 of WO2019043082, or fragments or variants thereof that produce functional amino acid sequences; (iv) a nucleic acid sequence having a coding sequence with at least 80%, at least 85%, at least 90%, at least 95%), at least 96%, at least 97%, at least 98%, or at least 99% sequence identity to the nucleic acid sequence as set forth in SEQ ID NO: 2, 4, 9, 11, or 14 of WO2019043082, or fragments thereof that produce functional amino acid sequences; (v) a nucleic acid sequence encoding an amino acid sequence as set forth in SEQ ID NO: 3, 5, 15, 42, or 43 of WO2019043082, or fragments or variants thereof that produce functional amino acid sequences; (vi) a nucleic acid sequence encoding an amino acid sequence with at least 80%, at least 85%, at least 90%, at least 95%, at least 96%, at least 97%, at least 98%, or at least 99% sequence identity to the amino acid sequence as set forth in SEQ ID NO: 3, 5, 15, 42, or 43 of WO2019043082, or fragments thereof. In a preferred embodiment, the restorer gene is located on an (monosomic) (alien) addition chromosome. In certain embodiments, the restorer gene is located on an additional chromosome to the plant's euploid number of chromosomes. In certain embodiments, the (dominant) male fertility restorer gene and at least one selection marker gene are on the additional chromosome. In a preferred embodiment, the restorer gene is located on a chromosome also carrying a selection marker, preferably a colour marker, preferably BLA, preferably BLA from Agropyron elongatum, Agropyron trichophorum, or Triticum monococcum. The selection marker may be in (close) linkage with the restorer gene, as described herein elsewhere (e.g. on the same chromosomal arm). In certain embodiments, the restorer gene and at least one selection marker gene are on the same side of the centromere of the chromosome. In certain embodiments, as used herein, the BLA gene has or comprises a sequence as set forth in (i) a nucleic acid sequence having a coding sequence of SEQ ID NO: 44 or 12 of WO2019043082, or fragments or variants thereof that produce functional amino acid sequences; (ii) a nucleic acid sequence having a coding sequence with at least 80%, at least 85%, at least 90%>, at least 95%), at least 96%>, at least 97%, at least 98%, or at least 99% sequence identity to the nucleic acid sequence of SEQ ID NO: 44 or 12 of WO2019043082, or fragments thereof that produce functional amino acid sequences; (iii) a nucleic acid sequence encoding an amino acid sequence of SEQ ID NO: 45 or 13 of WO2019043082, or fragments or variants thereof that produce functional amino acid sequences; (iv) a nucleic acid sequence encoding an amino acid sequence with at least 80%, at least 85%, at least 90%, at least 95%), at least 96%, at least 97%, at least 98%, or at least 99% sequence identity to the amino acid sequence of SEQ ID NO: 45 or 13 of WO2019043082, or fragments thereof.

As used herein, when reference is made to a plant (population) comprising a restorer gene, it is to be understood that such plant expresses or is capable of expressing (e.g. conditionally) the restorer gene product. Hereto, the restorer gene may be operably linked to a regulatory sequence, such as a promoter, which may be a native or endogenous promoter (or a promoter which is naturally linked to the restorer gene) or an artificial promoter (e.g. an exogenous promoter or a promoter which is not naturally linked to the restorer gene).

As used herein, “alien addition chromosome” can refer to a chromosome that is not native to the cereal plant in that it derived from a non-native chromosome (i.e., from a wholly different plant or different plant species, or from a wild relative of the cereal plant species) or at least a portion of the alien addition chromosome is derived from a non-native nucleic acid (e.g., at least the selection marker gene). With respect to the methods and cereal plants disclosed herein, the alien addition chromosome confers fertility to the cereal plant as it carries the male fertility restorer gene. Also, the alien addition chromosome confers a measurable phenotypic characteristic as it carriers a selection marker gene. In certain embodiments, the alien addition chromosome is monosomic, which results in a cereal plant with an odd number of chromosomes. In certain embodiments, the alien addition chromosome is translocated into the genome of the cereal plant, which can result in a cereal plant with an even number of chromosomes. In certain embodiments, the alien addition chromosome is disomic, which results in a cereal plant with an even number of chromosomes. In certain embodiments, the male fertility restorer gene of the alien species is located in a similar location as the male fertility gene of the cereal plant.

As used herein, the term “alien chromosome fragment” can refer to a portion of a chromosome that is derived from a non-native nucleic acid (e.g., at least the selection marker gene) or a native nucleic acid that is integrated into the genome in a location other than its natural location. With respect to the methods and cereal plants disclosed herein, the alien chromosome fragment confers fertility to the cereal plant as it carries the male fertility restorer gene. Also, the alien chromosome fragment confers a measurable phenotypic characteristic as it carriers a selection marker gene. In certain embodiments, the alien chromosome fragment is part of a homologous chromosome pair within the genome of the cereal plant.

As used herein, “non-native” or “exogenous” can refer to a nucleic acid or polypeptide sequence that is not found in a native nucleic acid or protein of the subject cereal plant. Non-native can refer to a naturally occurring nucleic acid or polypeptide sequence that comprises mutations, insertions and/or deletions. A non-native nucleic acid or polypeptide sequence may be linked to a naturally-occurring nucleic acid or polypeptide sequence (or a variant thereof) by genetic engineering to generate a chimeric nucleic acid and/or polypeptide sequence encoding a chimeric nucleic acid and/or polypeptide.

As used herein, the term “endogenous”, “native”, “original”, or “wild-type” refers to a naturally-occurring nucleic acid or polypeptide/protein. The native nucleic acid or protein may have been physically derived from a particular organism in which it is naturally occurring or may be a synthetically constructed nucleic acid or protein that is identical to the naturally-occurring nucleic acid or protein.

In certain embodiments of the disclosure, a “fertile plant” is a plant that produces viable male and female gametes and is self-fertile. Such a self-fertile plant can produce a progeny plant without the contribution from any other plant of a gamete and the genetic material contained therein. Other embodiments of the disclosure can involve the use of a plant that is not self-fertile because the plant does not produce male gametes, or female gametes, or both, that are viable or otherwise capable of fertilization.

As used herein, a “male sterile plant” is a plant that does not produce male gametes that are viable or otherwise capable of fertilization. As used herein, a “female sterile plant” is a plant that does not produce female gametes that are viable or otherwise capable of fertilization. It is recognized that male-sterile and female-sterile plants can be female-fertile and male-fertile, respectively. It is further recognized that a male fertile (but female sterile) plant can produce viable progeny when crossed with a female fertile plant and that a female-fertile (but male-sterile) plant can produce viable progeny when crossed with a male fertile plant. In certain embodiments, a male-sterile female parent is one in which no viable male can be produced if self-fertilized.

As used herein, the term “euploid” refers a normal complement of chromosomes. In certain embodiments, euploid refers to the number of chromosomes occurring in the wild-type plant.

Also provided herein, are selection marker genes that can be used to identify (male-)fertile or alternatively (male-)sterile plants cereal plants and/or seeds. The selection marker gene encodes a scoreable or screenable marker. In order to accurately identify the (male-)fertile/sterile plants, the selection marker must be associated with the male-fertility restorer gene. In certain embodiments, the marker gene and the male fertility restorer gene are located on the same side of centromere of the same chromosome, such that there is a significant reduction of while fertile seeds and plants from blue sterile seeds (due to mis-division). This is because there would be reduced chance of a mis-division causing the selection marker gene being separated or disassociated with the male fertility restorer gene (i.e., leading to two telocentric chromosomes with one carrying only the selection marker gene and the other carrying only the male fertility restorer gene).

For example, but not limitation, the selection marker gene can be a colour marker gene (e.g., seed, silks, husks, tassels, flowers, and/or grain), a plant height gene, a texture gene, an aroma gene, microsatellites (e.g., short tandem repeats, STRs, or simple sequence repeats, SSRs), restriction fragment length polymorphism (RFLP), random amplification of polymorphic DNA (RAPD), amplified fragment length polymorphism (AFLP), single nucleotide polymorphisms (SNPs), or a combination thereof.

In certain aspects, the selection marker is a colour marker (e.g., visual and/or fluorescent). When the selectable marker is a colour marker, it is possible to separate the cereal plants or seeds, depending on how the colour phenotype is expressed to determine which plants or seeds possess the male-fertility restorer gene. For examine, if the colour marker results in a seed having a specification (e.g., blue aleurone or other an endosperm colouring trait), it is possible to separate the seeds into coloured seeds (e.g., blue seeds) from which male-fertile plants (i.e., maintainer line) are developed, and natively coloured (e.g., red/white) seeds from which male-sterile plants (i.e., female line). The possibility to sort out the seeds of the male-sterile female line directly from the progeny simplifies the system and reduces to a great extent the production cost of the hybrid seeds. For example, a seed sorter would be able to detect the difference between the native colour and seeds expressing the colour marker.

In certain embodiments, the colour selection marker gene can come from, for example but not limited to, a blue aleurone gene (e.g., from Agropyron elongatum, Agropyron trichophorum, Triticum thaoudar, or Triticum monococcum).

In certain embodiments, the selection maker can be for example, without limitation, p-glucuronidase; uidA gene (GUS) (encoding an enzyme for which various chromogenic substrates are known (e.g., U.S. Pat. Nos. 5,268,463 and 5,599,670)); chloramphenicol acetyl transferase; alkaline phosphatase; anthocyanin/flavonoid polynucleotides (e.g., an R-locus polynucleotide (encoding a product that regulates the production of anthocyanin pigments (red colour) in plant tissues); genes controlling biosynthesis of flavonoid pigments (e.g., maize C1 and C2, the B gene, the pi gene, and the bronze locus genes); cyan fluorescent protein (CYP) gene; a the yellow fluorescent protein gene (YFP); red fluorescent protein gene (FP), yellow-green fluorescent protein (mNeonGreen), a lux gene (encoding luciferase); a green fluorescent protein (GFP), and Ds ed2 (Clontech Laboratories, Inc., Mountain View, Calif); p-lactamase gene encoding an enzyme for which various chromogenic substrates are known (e.g., PADAC, a chromogenic cephalosporin); a xylE gene (encoding a catechol dioxygenase that can convert chromogenic catechols); and a tyrosinase gene (encoding an enzyme capable of oxidizing tyrosine to DOPA and dopaquinone, which in turn condenses to form the easily detectable compound melanin). Also included are any selection markers the presence of which may be detected using, for example, X-ray film, scintillation counting, fluorescent spectrophotometry, low-light video cameras, photon counting detectors (e.g., cameras), and/or multiwell luminometry. Additional markers can be found at Yarranton, Curr Opin Biotech (1992) 3:506-11; Christopherson et al., Proc. Natl. Acad. Sci. USA (1992) 89:6314-8; Yao et al., Cell (1992) 71:63-72; Reznikoff, Mol Microbiol (1992) 6:2419-22; Hu et al., Cell (1987) 48:555-66; Brown et al., Cell (1987) 49:603-12; Figge et al., Cell (1988) 52:713-22; Deuschle et al., Proc. Natl. Acad. Sci. USA (1989) 86:5400-4; Fuerst et al., Proc. Natl. Acad. Sci. USA (1989) 86:2549-53; Deuschle et al., Science (1990) 248:480-3; Gossen, Ph.D. Thesis, University of Heidelberg (1993); Reines et al., Proc. Natl. Acad. Sci. USA (1993) 90: 1917-21; Labow et al., Mol Cell Biol (1990) 10:3343-56; Zambretti et al., Proc. Natl. Acad. Sci. USA (1992) 89:3952-6; Bairn et al., Proc. Natl. Acad. Sci. USA (1991) 88:5072-6; Wyborski et al., Nucleic Acids Res (1991) 19:4647-53; Hillen and Wissman, Topics Mol Struc Biol (1989) 10: 143-62; Degenkolb et al., Antimicrob Agents Chemother (1991) 35: 1591-5; Kleinschnidt et al., Biochemistry (1988) 27: 1094-104; Bonin, Ph.D. Thesis, University of Heidelberg (1993); Gossen et al., Proc. Natl. Acad. Sci. USA (1992) 89:5547-51; Oliva et al., Antimicrob Agents Chemother (1992) 36:913-9; Hlavka et al., Handbook of Experimental Pharmacology (1985), Vol. 78 (Springer-Verlag, Berlin); Gill et al., Nature (1988) 334:721-4; all of which are incorporated by reference herein in their entirety for all intended purposes.

“Linkage” refers to the tendency for alleles to segregate together more often than expected by chance if their transmission was independent. Typically, linkage refers to alleles on the same chromosome. Genetic recombination occurs with an assumed random frequency over the entire genome. Genetic maps are constructed by measuring the frequency of recombination between pairs of traits or markers. The closer the traits or markers are to each other on the chromosome, the lower the frequency of recombination, and the greater the degree of linkage. Traits or markers are considered herein to be linked if they generally co-segregate. A 1/100 probability of recombination per generation is defined as a genetic map distance of 1.0 centiMorgan (1.0 cM). The term “linkage disequilibrium” refers to a non-random segregation of genetic loci or traits (or both). In either case, linkage disequilibrium implies that the relevant loci are within sufficient physical proximity along a length of a chromosome so that they segregate together with greater than random (i.e., non-random) frequency. Markers that show linkage disequilibrium are considered linked. Linked loci co-segregate more than 50% of the time, e.g., from about 51% to about 100% of the time. In other words, two markers that co-segregate have a recombination frequency of less than 50% (and by definition, are separated by less than 50 cM on the same linkage group.) As used herein, linkage can be between two markers, or alternatively between a marker and a locus affecting a phenotype. A marker locus can be “associated with” (linked to) a trait. The degree of linkage of a marker locus and a locus affecting a phenotypic trait is measured, e.g., as a statistical probability of co-segregation of that molecular marker with the phenotype (e.g., an F statistic or LOD score).

The term “mutation” or “mutated” as used herein refers to a gene or protein product thereof which is altered or modified such that the function normally attributed to the gene or protein product thereof is altered, or alternatively such that the expression, stability, and/or activity normally associated with the gene or protein product thereof is altered. Typically, a mutation as referred to herein results in a phenotypic effect, such as male sterility, as described herein elsewhere. It will be understood that a mutation in a gene or protein product thereof is referred to in comparison with a gene or protein product thereof not having such mutation, such as a wild type or endogenous gene or protein product thereof. Typically, a mutation refers to a modification at the DNA level, and includes changes in the genetics and/or epigenetics. An alteration in the genetics may include an insertion, a deletion, an introduction of a stop codon, a base change (e.g. transition or transversion), or an alteration in splice junctions. These alterations may arise in coding or non-coding regions (e.g. promoter regions, exons, introns or splice junctions) of the endogenous DNA sequence. For example, an alteration in the genetics may be the exchange (including insertions, deletions) of at least one nucleotide in the endogenous DNA sequence or in a regulatory sequence of the endogenous DNA sequence. If such a nucleotide exchange takes place in a promoter, for example, this may lead to an altered activity of the promoter, since, for example, cis-regulator elements are modified such that the affinity of a transcription factor to the mutated cis-regulatory elements is altered in comparison to the wild-type promoter, so that the activity of the promoter with the mutated cis-regulatory elements is increased or reduced, depending upon whether the transcription factor is a repressor or inductor, or whether the affinity of the transcription factor to the mutated cis-regulatory elements is intensified or weakened. If such a nucleotide exchange occurs, e.g., in an encoding region of the endogenous DNA sequence, this may lead to an amino acid exchange in the encoded protein, which may produce an alteration in the activity or stability of the protein, in comparison to the wild-type protein. An alteration in the epigenetics may take place via an altered methylation pattern of the DNA. In certain embodiments, a mutation as referred to herein relates to the insertion of one or more nucleotides in a gene. In certain embodiments, a mutation as referred to herein relates to the deletion of one or more nucleotides in a gene. In certain embodiments, the mutation as referred to herein relates to the deletion as well as the insertion of one or more nucleotides. In certain embodiments, certain nucleotide stretches, such as for instance encoding a particular protein domain are deleted. In certain embodiments, a mutation as referred to herein relates to the exchange of one or more nucleotides in a gene by different nucleotides. In certain embodiments, the mutation is a nonsense mutation (i.e. the mutation results in the generation of a stop codon in a protein encoding sequence). In certain embodiments, the mutation is a frameshift mutation (i.e. an insertion or deletion of one or more nucleotides (not equal to three or a product thereof) in a protein encoding sequence). In certain embodiments, the mutation results in a truncated protein product. In certain embodiments, the mutation results in an N-terminally truncated protein product. In certain embodiments, the mutation results in a C-terminally truncated protein product. In certain embodiments, the mutation results in an N-terminally and C-terminally truncated protein product. In certain embodiments, the mutation results in an altered splice site (such as an altered splice donor and/or splice acceptor site). In certain embodiments, the mutation is in an exon. In certain embodiments, the mutation is in an intron. In certain embodiments, the mutation is in a regulatory sequence, such as a promoter. In certain embodiments, the mutation results in a codon encoding a different amino acid. In certain embodiments, the mutation results in the insertion or deletion of one or more codons (i.e. nucleotide triplets). In certain embodiments, the mutation is a knockout mutation. Both frameshift and nonsense mutations can in certain embodiments be considered as knockout mutations, in particular if the mutation is present in an early exon. A knockout mutation as used herein preferably means that a functional gene product, such as a functional protein, is not produced anymore. In particular, frameshift and nonsense mutations will lead to premature termination of protein translation, such that a truncated protein will result, which often lacks the required stability and/or activity to perform the function naturally attributed to it. In certain embodiments, the mutation is a knockdown mutation. In contrast to a knockout mutation, a knockdown mutation results in a decreased activity, stability, and/or expression rate of the native functional gene product, such as a protein, and thereby ultimately in a decreased functionality. For instance, mutations in promoter regions affecting transcriptional activator binding (or other regulatory sequences), in particular reducing transcription rate, can be considered knockdown mutations. Also mutations negatively affecting protein stability (such as to increase ubiquitination and subsequent protein degradation) can be considered knockdown mutations). In addition, mutations negatively affecting protein activity (such as binding strength or enzymatic activity) can be considered knockdown mutations. It will be understood that the mutations described herein according to the invention confer (genetic) male sterility, as described herein elsewhere. While the mutation envisaged herein may be non-naturally occurring, this need not necessarily be the case. In certain embodiments, a wild type/endogenous allele is replaced by a mutated allele, preferably all wild type/endogenous alleles are replaced by a mutated allele. Replacement can be effected by any means known in the art, as also described herein elsewhere. Replacement, as used herein also includes (direct) mutagenesis of the wild type/endogenous allele(s) at its native genomic locus. Accordingly, in certain embodiments, a wild type/endogenous allele is mutated, as described herein elsewhere, preferably all wild type/endogenous alleles are mutated. The skilled person will understand that only one copy of a wild type/endogenous allele may be mutated and that homozygosity (if so desired) may be obtained by selfing and subsequent selection. In certain embodiments, a reduced number of wild type/endogenous alleles is present (i.e. the wild type/endogenous allele is heterozygous). In certain embodiments, a wild type/endogenous allele is knocked out, preferably all wild type/endogenous alleles are knocked out, and a mutated allele is transgenically introduced, transiently or genomically integrated, preferably genomically integrated. In certain embodiments, a wild type/endogenous allele is knocked out, preferably all wild type/endogenous alleles are knocked out, and is transgenically replaced by a mutated allele (at the native genomic location of the wild type allele). The skilled person will understand that only one copy of a wild type/endogenous allele may be knocked out and that homozygosity (if so desired) may be obtained by selfing and subsequent selection.

Mutations as described herein may be introduced by mutagenesis, which may be performed in accordance with any of the techniques known in the art. As used herein, “mutagenization” or “mutagenesis” includes both conventional mutagenesis and location-specific mutagenesis or “genome editing” or “gene editing”. In conventional mutagenesis, modification at the DNA level is not produced in a targeted manner. The plant cell or the plant is exposed to mutagenic conditions, such as TILLING, via UV light exposure or the use of chemical substances (Till et al., 2004). An additional method of random mutagenesis is mutagenesis with the aid of a transposon. Location-specific mutagenesis, such as gene editing, enables the introduction of modification at the DNA level in a target-oriented manner at predefined locations in the DNA. For example, TALENS, meganucleases, homing endonucleases, zinc finger nucleases, or a CRISPR/Cas system may be used for this.

In certain embodiments, the mutations as defined herein are homozygous. Accordingly, in diploid plants the two alleles are identical (at least with respect to the particular mutation), in tetraploid plants the four alleles are identical, and in hexaploid plants the six alleles are identical with respect to the mutation or marker. In certain embodiments, the mutation/marker as defined herein is heterozygous. Accordingly, in diploid plants the two alleles are not identical, in tetraploid plants the four alleles are not identical (for instance only one, two, or three alleles comprise the specific mutation/marker), and in hexaploid plants the six alleles are not identical with respect to the mutation or marker (for instance only one, two, three, four or five alleles comprise the specific mutation/marker). Similar considerations apply in case of pseudopolyploid pants.

As used herein, the terms “phenotype,” “phenotypic trait” or “trait” refer to one or more traits of a plant or plant cell. The phenotype can be observable to the naked eye, or by any other means of evaluation known in the art, e.g., microscopy, biochemical analysis, or an electromechanical assay. In some cases, a phenotype is directly controlled by a single gene or genetic locus (i.e., corresponds to a “single gene trait”). In the case of haploid induction use of color markers, such as R Navajo, and other markers including transgenes visualized by the presences or absences of color within the seed evidence if the seed is an induced haploid seed. The use of R Navajo as a color marker and the use of transgenes is well known in the art as means to detect induction of haploid seed on the female plant. In other cases, a phenotype is the result of interactions among several genes, which in some embodiments also results from an interaction of the plant and/or plant cell with its environment.

As used herein, the term “homozygote” refers to an individual cell or plant having the same alleles at one or more or all loci. When the term is used with reference to a specific locus or gene, it means at least that locus or gene has the same alleles. As used herein, the term “homozygous” means a genetic condition existing when identical alleles reside at corresponding loci on homologous chromosomes. Accordingly, for diploid organisms, the two alleles are identical, for tetraploid organisms, the 4 alleles are identical, etc. As used herein, the term “heterozygote” refers to an individual cell or plant having different alleles at one or more or all loci. When the term is used with reference to a specific locus or gene, it means at least that locus or gene has different alleles. Accordingly, for diploid organisms, the two alleles are not identical, for tetraploid organisms, the 4 alleles are not identical (i.e. at least one allele is different than the other alleles), etc. As used herein, the term “heterozygous” means a genetic condition existing when different alleles reside at corresponding loci on homologous chromosomes. In certain embodiments, the proteins, genes, or coding sequences as described herein is/are homozygous. In certain embodiments, the proteins, genes, or coding sequences as described herein are heterozygous. In certain embodiments, proteins, genes, or coding sequence alleles as described herein is/are homozygous. In certain embodiments, the proteins, genes, or coding sequence alleles as described herein are heterozygous. It will be understood that homozygosity or heterozygosity preferably relates to at least a gene, i.e. the locus comprising the gene (or coding sequence derived thereof, or protein encoded thereby). However, more specifically, homozygosity or heterozygosity may equally refer to a particular mutation, such as a mutation described herein. Accordingly, a particular mutation can be considered to be homozygous (i.e. all alleles carry the mutation), whereas for instance the remainder of the gene, coding sequence, or protein may comprise differences between alleles. In certain embodiments, the mutations as defined herein are recessive. In certain embodiments, the mutations as defined herein are recessive and homozygous.

As used herein “chemical hybridization agent” or “CHA” refers to chemical agents which are used to induce (male) sterility in plants, and hence can be used as a tool for hybrid plant production, in particular in self-pollinating plants. Chemicals used to induce male sterility include chemical hybridizing agents (CHAs), male gametocides and pollen suppressants (Razzaq et al, 2015, Seed Technology, 37(1): 23-31; Sleper and Poehlman, 2006; Kaul, 2012). Male sterility induced through CHAs is an important tool for the exploitation of hybrid vigor in field crops (Cheng et al., 2013). Accurate CHA dosages at critical stages of head development could induce complete male sterility (Cross and Ladyman, 1991). Selective CHAs have been exploited in many breeding lines, eliminating lengthy procedures involved in cytoplasmic male sterility (CMS) and maintenance of fertility restoration, and mitigating the negative impact on performance of inbred lines due to induction of CMS from other species (Cisar and Cooper, 2003). CHAs can also be utilized for assessing large numbers of genotypes for general and specific combining ability during the early evaluation phase of the candidate inbred lines, and can be exploited as a substitute to hand emasculation in interspecific and intervarietal crosses, as well as recurrent back crosses. The present invention preferably avoids the use of CHA. Accordingly, in certain embodiments the methods of the invention as described herein do not involve the use of CHA.

As used herein, the term “heterosis” has its ordinary meaning in the art, and may also be referred to as hybrid vigor or outbreeding enhancement. By means of further guidance, and without limitation, heterosis refers to the improved or increased function of any biological quality (such as one or more agronomic or physiologic characteristics or traits) in a (hybrid) offspring. An offspring is heterotic if one or more of its traits are enhanced as a result of mixing the genetic contributions of its parents. These effects can be due to Mendelian or non-Mendelian inheritance. Heterosis may result in the offspring of a cross of (inbred) lines. Heterosis may manifest in any one or more plant characteristics and may therefore be evaluated by testing, analysing, or determining such characteristic, either quantitatively, or qualitatively. Typically, such characteristic is compared to the corresponding characteristic in one or both of the parent plants. Heterosis is performance of F1 compared to the average of the parents individual value. This is called “mid-parent” heterosis. “Best-parent heterosis” is performance compared to the value of the best parent. “Commercial heterosis” relates to performance of F1 compared to best commercial comparison. By means of example, mid-parent and best-parent heterosis (for a specific characteristic or trait) can be quantified respectively as follows:

% Ht=(F1−M.P)/M.P×100

Ht=Heterosis, M.P=Mid parent

% Hbt=(F1−B.P)/B.P×100

Hbt=Heterobeltiosis, B.P=Better parent

As used herein, the terms “combining ability”, “general combining ability” or “GCA”, and “specific combining ability” or “SCA” have their ordinary meaning in the art. By means of further guidance, and without limitation, combining ability refers to the parents ability to combine among each other during hybridization process such that desirable genes or characters are transmitted to their progenies (Fasahat et al., DOI: 10.15406/bbij.2016.04.00085). In another definition, combining ability is an estimation of the value of genotypes on the basis of their offspring performance in some definite mating design (Allard R W. Principles of Plant Breeding, John Wiley and Sons Inc, New York, USA; 1960). It can seldom be envisaged only based on parental phenotype and thus it is measured by progeny testing. When parental plants produce potent offspring, they are said to have good combining ability Vasal S K, Cordova H, Pandey S, et al. Tropical maize and heterosis. CIMMYT research highlights, Mexico, DF, CIMMYT. 1986). At first, combining ability was a general concept used collectively for classifying an inbred line respective to its cross performance but was later amended. Two concepts of general combining ability (GCA) and specific combining ability (SCA) are important for on inbred line evaluation and population development in crop breeding. Sprague and Tatum (General versus specific combining ability in single crosses of corn. Journal of the American Society of Agronomy. 1942; 34:923-932) defined GCA as the average performance of a genotype in a series of hybrid combinations. They defined SCA as those cases in which certain hybrid combinations perform better or poorer than would be expected on the basis of the average performance of the parental inbred lines. Parents showing a high average combining ability in crosses are considered to have good GCA while if their potential to combine well is bounded to a particular cross, they are considered to have good SCA. From a statistical point of view, the GCA is a main effect and the SCA is an interaction effect. GCA is owing to the activity of genes which are largely additive in their effects as well as additive×additive interactions. Specific combining ability is regarded as an indication of loci with dominance variance (non-additive effects) and all the three types of epistatic interaction components if epistasis were present. They include additive×dominance and dominance×dominance interactions. The combining ability of lines for main characteristics is estimated by examining a set of designed progeny in good trial design accompanied by statistical analysis. Furthermore, parent selection for combining ability is conducted through growing and evaluating the progenies. Several techniques are available for the estimation of combining ability (Fasahat et al., DOI: 10.15406/bbij.2016.04.00085). These include top cross suggested by Davis (Report of Plant breeder. Annual Report of the Puerto Rico Agriculture Experimental Station. 1927. p. 14-15) and developed by Jenkins and Brunaon, (A method of testing inbred line of maize in cross bed combinations. J Ann Sci Agron. 1932; 24:523-530) poly cross technique proposed by Tysdal et al., (Alfalfa breeding. Coll Agric Univ Nebraska Agric. Exp Sta Res Bull. 1942; 124:1-46) diallel cross analysis by Griffing, (Concept of general and specific combining ability in relation to diallel crossing system. Australian Journal of Biological Sciences. 1956b; 9(4):463-493), line×tester analysis by Kempthorne, (An introduction of genetics statistics. John Wiley and Sons, New York, USA. 1957 p. 458-471), partial diallel cross by Kempthorne & Curnow, (The partial diallel cross. Biometrics. 1961; 17(2):229-240), North Carolina design by Comstock & Robinson, (The components of genetic variance in populations of biparental progenies and their uses in estimating the average degree of dominance. Biometrics. 1948; 4(4):254-266.), and triallel cross by Rawlings & Cockerham (Analysis of double cross hybrid populations. Biometrics. 1962; 18:229-244) are used to estimate combining ability.

The evaluation of heterosis according to certain embodiments of the invention, as does the evaluation of (general or specific) combining ability or more in general the evaluation of hybrid test crosses, entails the evaluation of one or more plant characteristics, in particular one or more agronomic, physiologic, or quality characteristics. Such characteristics may be compared to the corresponding characteristics in one or both parents, as is known in the art (mid-parent or best-parent).

By means of example, and without limitation, relevant plant characteristics, include a variety of characteristics such as agronomic, physiologic, or quality characteristics. Some exemplary agronomic, physiologic, or quality characteristics or traits include: seed yield, plant height, number of (productive) tillers per plant, spike length, number of spikelets per spike, number of grains per spike, grain yield per plant, grain yield, total (above ground) biomass yield, disease resistance, drought resistance, stress resistance, days to anthesis, harvest index, straw yield, grain weight per spike, thousand seed weight, grain volume weight, tillering, hectoliter weight, frost damage, date of ear emergence, lodging, seed hardness, seed protein content, seed total gluten content, seed gluten index, seed moisture content, days to heading, etc. In certain preferred embodiments, the (agronomic, physiological, or quality) characteristic is seed (or grain) yield. The skilled person will understand that seed yield may be expressed in a number of different ways, such as seed yield per plant, seed yield per growth area, seed yield per spike, seed yield per unit of (total or above ground) biomass, etc.

An agronomically relevant characteristic in the context of the present invention may be associated with a phenotype of a plant, which exhibits one or more novel or optimized trait(s) that provide an improved agricultural performance with respect to e.g. yield, biomass, architecture, morphology, fertility, pollen shedding, nutrient partitioning, photosynthesis, carbon sequestration, disease resistance, abiotic and biotic stress tolerance, herbicide tolerance, hormone signaling, and other trait categories. A phenotype may be caused by any one or a combination of one or more allelic variations in one or more coding, non-coding or regulatory regions of the genetic material of the plant. The modifications may be associated in terms of spatial proximity or genomic context or they may be completely unrelated. An phenotype may thus exhibit one or more polygenic traits.

Hybrid test crosses according to the invention as described herein, refer to the set-up of particular breeding schemes in order to evaluation hybrid breeding, such as by evaluating heterosis or (general and/or specific) combining ability. The line×tester is the most widely used mating design for hybrid development (Fasahat et al., DOI: 10.15406/bbij.2016.04.00085). Line×tester analysis which involves ‘l’ lines and ‘t’ testers is an extension of the analysis of two factor factorial experiment introduced by Fisher and Yates (Fisher R A. The arrangement of field experiments. Journal of Ministry of Agriculture. 1926; 33:503-513; Yates F. Complex experiments. Supplement to the Journal of the Royal Statistical Society. 1935; 2:181-223). In this design, full-sib progenies are generated through crossing ‘l’ lines to ‘t’ testers. Then, developed progenies as well as parents, are evaluated in developed field trials.

A tester is a genotype (line, cultivar, variety) that is used to identify superior germplasm in accordance with breeding objectives in a hybrid-oriented program. A tester line as defined by different researchers (Matzinger D F. Comparison of three types of testers for the evaluation of inbred lines of corn. Agronomy Journal. 1953; 45:493-495; Rawlings J O, Thompson D L. Performance level as criterion for the choice of maize testers. Crop Science. 1962; 2:217-220; Allison J C S, Curnow R W. On the choice of tester parent for the breeding of synthetic varieties of maize (Zea mays L.). Crop Science. 1966; 6(6):541-544) is the one that have simplicity in use, provide information that classifies relative performance of lines into heterotic groups or heterotic patterns, and maximize the expected mean yield. Heterotic patterns are populations or lines with high mean heterosis as a result of high genetic divergence, different in allele frequency and have high combining ability. The materials which can be considered as testers consist of inbred lines, single cross hybrids and heterogeneous materials, which encompass open pollinated varieties, synthetic or populations. These materials fall into two broad groups namely broad genetic base (heterogeneous materials) as well as narrow genetic base testers (single crosses and inbred lines). A broad genetic based tester is considered for GCA selection while a narrow genetic based tester is used for SCA selection. Testers can be selected according to the program goals and the types of hybrids developed. The initial tester is usually chosen based on experience with most commercial hybrid improvement programs using inbred parents with proven hybrid performance. The choice is made through using information on the pedigree of the genotypes being tested along with the knowledge of the performance of the tester. No single tester fulfils all these needs for all circumstances as the value of a tester is specified to a considerable proportion by the use to be made of a special group of lines. In a reciprocal recurrent selection (RRS), a suitable tester is selected from a population of the opposite heterotic group. If the objective is to evaluate lines of unknown origin at least two testers from established heterotic groups are employed as suitable testers to determine heterotic orientation of new lines. At least two elite lines from opposite heterotic groups or showing high levels of heterosis between them can be used as testers when the objective is to divide a broad-baas used herein sed population into two heterotic groups.

As used herein, the terms “male pool” and “female pool” relate respectively to collections of populations, lines, cultivars, or varieties which are typically used as male or female plants, i.e. which typically provide respectively the male or female gametes in crosses. Designation of a particular plant population, line, cultivar, or variety in the male or female pool typically resides in their suitability for use as a male or female plant, respectively, as is known by the skilled person in the art. By means of example, and without limitation, such suitability may be assigned based on relevant characteristics associated with or attributed to development and functionality of male or female gametes or reproductive organs. For instance in cereals, such as from the genus Triticum, (adequate) anther extrusion may quality a particular line for inclusion in the male pool. In general, underlying assignment to the male pool is the line's ability to disperse sufficient amount of pollen to ensure high level of fertilization of the sterile female. Important characters include, but not limited to, extrusion of anthers, release of pollen after and not before anthers are extruded, high volume of pollen produced, staggered flowering to increase the period of pollen dispersal, dispersal of viable pollen, and pollen morphology facilitating spreading by wind. By means of example and without limitation, for the female pool, wide “gaping” (opening of flower), duration of gaping, duration of stigma receptivity, large number of florets produced per unit area of land are relevant characteristics.

“Transgenic” or “genetically modified organisms” (GMOs) as used herein are organisms whose genetic material has been altered using techniques generally known as “recombinant DNA technology”. Recombinant DNA technology encompasses the ability to combine DNA molecules from different sources into one molecule ex vivo (e.g. in a test tube). The term “transgenic” here means genetically modified by the introduction of a non-endogenous nucleic acid sequence. Typically a species-specific nucleic acid sequence is introduced in a form, arrangement or quantity into the cell in a location where the nucleic acid sequence does not occur naturally in the cell. This terminology generally does not cover organisms whose genetic composition has been altered by conventional cross-breeding or by “mutagenesis” breeding, as these methods predate the discovery of recombinant DNA techniques. “Non-transgenic” as used herein refers to plants and food products derived from plants that are not “transgenic” or “genetically modified organisms” as defined above.

“Gene editing” or “genome editing” refers to genetic engineering in which in which DNA or RNA is inserted, deleted, modified or replaced in the genome of a living organism. Gene editing may comprise targeted or non-targeted (random) mutagenesis. Targeted mutagenesis may be accomplished for instance with designer nucleases, such as for instance with meganucleases, zinc finger nucleases (ZFNs), transcription activator-like effector-based nucleases (TALEN), and the clustered regularly interspaced short palindromic repeats (CRISPR/Cas9) system. These nucleases create site-specific double-strand breaks (DSBs) at desired locations in the genome. The induced double-strand breaks are repaired through nonhomologous end-joining (NHEJ) or homologous recombination (HR), resulting in targeted mutations or nucleic acid modifications. The use of designer nucleases is particularly suitable for generating gene knockouts or knockdowns. In certain embodiments, designer nucleases are developed which specifically introduce one or more of the molecular marker (allele) according to the invention as described herein. Delivery and expression systems of designer nuclease systems are well known in the art.

The term “gene” when used herein refers to a polymeric form of nucleotides of any length, either ribonucleotides or desoxyribonucleotides. The term includes double- and single-stranded DNA and RNA. It also includes known types of modifications, for example, methylation, “caps”, substitutions of one or more of the naturally occurring nucleotides with an analog. Preferably, a gene comprises a coding sequence encoding the herein defined polypeptide. A “coding sequence” is a nucleotide sequence which is transcribed into mRNA and/or translated into a polypeptide when placed or being under the control of appropriate regulatory sequences. The boundaries of the coding sequence are determined by a translation start codon at the 5′-terminus and a translation stop codon at the 3′-terminus. A coding sequence can include, but is not limited to mRNA, cDNA, recombinant nucleic acid sequences or genomic DNA, while introns may be present as well under certain circumstances.

In an aspect, the invention relates to a method for generating a hybrid plant, comprising crossing one or more first plant or plant population, line, cultivar, or variety with one or more second plant or plant population, line, cultivar, or variety wherein said first or second plant or plant population, line, cultivar, or variety is a (genetically) male sterile plant or plant population, line, cultivar, or variety. Preferably the first and second plant are from the genus Triticum, preferably Triticum aestivum.

In an aspect, the invention relates to a method for generating a hybrid plant, comprising
(a) crossing a plant or plant population, line, cultivar, or variety comprising a (genetic) (male) sterility restorer gene, preferably comprising a (homozygous) ms (such as ms1) mutation (e.g. deletion or knockout), with a (genetic) (male) sterile plant, preferably comprising a (homozygous) ms (such as ms1) mutation (e.g. deletion or knockout);
(b) selecting (genetic) (male) sterile offspring, e.g. seed (not containing the restorer gene), such as for instance based on a selection marker, such as BLA (linked to the restorer gene, as described herein elsewhere), wherein white or non-blue seeds are selected;
(c) crossing said offspring as a first plant or plant population, line, cultivar, or variety with one or more second plant or plant population, line, cultivar or variety (which is a (genetically) (male) fertile plant or plant population, line, cultivar, or variety) to generate a hybrid plant (or part thereof, e.g. seed) or plant population;
(d) optionally further determining one or more (agronomic, physiologic, or quality) characteristics or traits of said hybrid plant or plant population (so as to evaluate the plant hybrid test cross or to determine the (general and/or specific) combining ability or heterosis of said particular plant or combination of parent plants).

In an aspect, the invention relates to a method for generating a hybrid plant, comprising
(a) self-fertilizing a plant or plant population, line, cultivar, or variety comprising a (genetic) (male) sterility restorer gene, preferably comprising a (homozygous) ms (such as ms1) mutation (e.g. deletion or knockout);
(b) selecting (genetic) (male) sterile offspring, e.g. seed (not containing the restorer gene), such as for instance based on a selection marker, such as BLA (linked to the restorer gene, as described herein elsewhere), wherein white or non-blue seeds are selected;
(c) crossing said offspring as a first plant or plant population, line, cultivar, or variety with one or more second plant or plant population, line, cultivar or variety (which is a (genetically) (male) fertile plant or plant population, line, cultivar, or variety) to generate a hybrid plant (or part thereof, e.g. seed) or plant population;
(d) optionally further determining one or more (agronomic, physiologic, or quality) characteristics or traits of said hybrid plant or plant population (so as to evaluate the plant hybrid test cross or to determine the (general and/or specific) combining ability or heterosis of said particular plant or combination of parent plants).

The methods of the present invention are of particular interest in the context of testing combining ability of plants or plant populations. Accordingly, in an aspect, the invention relates to a method for testing, analyzing, evaluating, or determining (general and/or specific) combining ability, comprising crossing one or more first plant or plant population, line, cultivar, or variety with one or more second plant or plant population, line, cultivar, or variety wherein said first or second plant or plant population, line, cultivar, or variety is a (genetically) male sterile plant or plant population, line, cultivar, or variety. Preferably the first and second plant are from the genus Triticum, preferably Triticum aestivum.

The methods of the present invention make it possible to determine heterosis for a combination of plants or plant populations. Accordingly, in an aspect, the invention relates to a method for testing, analyzing, evaluating, or determining heterosis, comprising crossing one or more first plant or plant population, line, cultivar, or variety with one or more second plant or plant population, line, cultivar, or variety wherein said first or second plant or plant population, line, cultivar, or variety is a (genetically) male sterile plant or plant population, line, cultivar, or variety. Preferably the first and second plant are from the genus Triticum, preferably Triticum aestivum.

The methods of the invention can be used for test crossing for hybrid plants. In an aspect, the invention relates to a method for plant hybrid test crossing, comprising crossing one or more first plant or plant population, line, cultivar, or variety with one or more second plant or plant population, line, cultivar, or variety wherein said first or second plant or plant population, line, cultivar, or variety is a (genetically) male sterile plant or plant population, line, cultivar, or variety. Preferably the first and second plant are from the genus Triticum, preferably Triticum aestivum.

The methods of the invention typically involve analyzing the offspring of the crossing of the first and second plant or plant population. In certain embodiments, the methods of the invention as described herein further comprise harvesting the first and/or second plants or plant parts thereof, preferably seeds.

The analysis of the offspring can be performed in different ways. In certain embodiments, the methods of the invention as described herein further comprise determining the yield, such as (biomass) yield of the plants or plant parts, preferably grains or seeds. In certain embodiments, the methods of the invention as described herein further comprise testing, analyzing, evaluating, or determining one or more (agronomic, physiologic, or quality) characteristics or traits (in the (F1) progeny). In certain embodiments, the methods of the invention as described herein further comprise testing, analyzing, evaluating, or determining heterosis or one or more (agronomic, physiologic, or quality) characteristics or traits (in or of the (F1) progeny or of the first and second plant). In certain embodiments, the methods of the invention as described herein further comprise testing, analyzing, evaluating, or determining general and/or specific combining ability or one or more agronomic (agronomic, physiologic, or quality) or traits (in or of the (F1) progeny or of the first and second plant).

The methods of the present invention can be achieved in practice by specific sowing methods. In certain embodiments, the methods of the invention as described herein comprise sowing seeds of said one or more first plant or plant population, line, cultivar, or variety or planting plants of said one or more first plant or plant population, line, cultivar, or variety in one or more parallel row; and sowing seeds of said one or more second plant or plant population, line, cultivar, or variety or planting plants of said one or more second plant or plant population, line, cultivar, or variety in one or more parallel row flanking or flanked by said one or more parallel row of said one or more first plant or plant population, line, cultivar, or variety. Planting schemes in certain embodiments are as defined herein elsewhere.

In an aspect, the invention relates to a method for testing, analyzing, evaluating, or determining (general and/or specific) combining ability, providing an (F1) progeny plant or plant population resulting from crossing one or more first plant or plant population, line, cultivar, or variety with one or more second plant or plant population, line, cultivar, or variety wherein said first or second plant or plant population, line, cultivar, or variety is a (genetically) male sterile plant or plant population, line, cultivar, or variety; and test, analyze, evaluate, or determine general and/or specific combining ability or one or more (agronomic, physiologic, or quality) characteristics or traits (in or of the (F1) progeny or of the first and second plant). Preferably the first and second plant are from the genus Triticum, preferably Triticum aestivum. In certain embodiments, seeds of said one or more first plant or plant population, line, cultivar, or variety or plants of said one or more first plant or plant population, line, cultivar, or variety have been sown or planted in one or more parallel row; and seeds of said one or more second plant or plant population, line, cultivar, or variety or plants of said one or more second plant or plant population, line, cultivar, or variety have been sown or planted in one or more parallel row flanking or flanked by said one or more parallel row of said one or more first plant or plant population, line, cultivar, or variety. Planting schemes in certain embodiments are as defined herein elsewhere.

In an aspect, the invention relates to a method for testing, analyzing, evaluating, or determining heterosis, comprising providing an (F1) progeny plant or plant population resulting from crossing one or more first plant or plant population, line, cultivar, or variety with one or more second plant or plant population, line, cultivar, or variety wherein said first or second plant or plant population, line, cultivar, or variety is a (genetically) male sterile plant or plant population, line, cultivar, or variety; and test, analyze, evaluate, or determine heterosis or one or more (agronomic, physiologic, or quality) characteristics or traits (in or of the (F1) progeny or of the first and second plant). Preferably the first and second plant are from the genus Triticum, preferably Triticum aestivum. In certain embodiments, seeds of said one or more first plant or plant population, line, cultivar, or variety or plants of said one or more first plant or plant population, line, cultivar, or variety have been sown or planted in one or more parallel row; and seeds of said one or more second plant or plant population, line, cultivar, or variety or plants of said one or more second plant or plant population, line, cultivar, or variety have been sown or planted in one or more parallel row flanking or flanked by said one or more parallel row of said one or more first plant or plant population, line, cultivar, or variety. Planting schemes in certain embodiments are as defined herein elsewhere.

In an aspect, the invention relates to a method for evaluating plant hybrid test crosses, comprising providing an (F1) progeny plant or plant population resulting from crossing one or more first plant or plant population, line, cultivar, or variety with one or more second plant or plant population, line, cultivar, or variety wherein said first or second plant or plant population, line, cultivar, or variety is a (genetically) male sterile plant or plant population, line, cultivar, or variety; and test, analyze, evaluate, or determine general and/or specific combining ability or heterosis, or one or more (agronomic, physiologic, or quality) characteristics or traits (in or of the (F1) progeny or of the first and second plant). Preferably the first and second plant are from the genus Triticum, preferably Triticum aestivum. In certain embodiments, seeds of said one or more first plant or plant population, line, cultivar, or variety or plants of said one or more first plant or plant population, line, cultivar, or variety have been sown or planted in one or more parallel row; and seeds of said one or more second plant or plant population, line, cultivar, or variety or plants of said one or more second plant or plant population, line, cultivar, or variety have been sown or planted in one or more parallel row flanking or flanked by said one or more parallel row of said one or more first plant or plant population, line, cultivar, or variety. Planting schemes in certain embodiments are as defined herein elsewhere.

In an aspect, the invention relates to a method for sowing or planting, comprising sowing seeds of one or more first plant or plant population, line, cultivar, or variety or planting plants of one or more first plant or plant population, line, cultivar, or variety in one or more parallel row; sowing seeds of one or more second plant or plant population, line, cultivar, or variety or planting plants of one or more second plant or plant population, line, cultivar, or variety in one or more parallel row flanking or flanked by said one or more parallel row of said one or more first plant or plant population, line, cultivar, or variety; wherein said first or second plant or plant population, line, cultivar, or variety is a (genetically) male sterile plant or plant population, line, cultivar, or variety. Preferably the first and second plant are from the genus Triticum, preferably Triticum aestivum.

In an aspect, the invention relates to a method for sowing or planting for generating a hybrid plant, comprising sowing seeds of one or more first plant or plant population, line, cultivar, or variety or planting plants of one or more first plant or plant population, line, cultivar, or variety in one or more parallel row; sowing seeds of one or more second plant or plant population, line, cultivar, or variety or planting plants of one or more second plant or plant population, line, cultivar, or variety in one or more parallel row flanking or flanked by said one or more parallel row of said one or more first plant or plant population, line, cultivar, or variety; wherein said first or second plant or plant population, line, cultivar, or variety is a (genetically) male sterile plant or plant population, line, cultivar, or variety. Preferably the first and second plant are from the genus Triticum, preferably Triticum aestivum.

In an aspect, the invention relates to a method for sowing or planting for testing, evaluating, analyzing, or determining (general and/or specific) combining ability, comprising sowing seeds of one or more first plant or plant population, line, cultivar, or variety or planting plants of one or more first plant or plant population, line, cultivar, or variety in one or more parallel row; sowing seeds of one or more second plant or plant population, line, cultivar, or variety or planting plants of one or more second plant or plant population, line, cultivar, or variety in one or more parallel row flanking or flanked by said one or more parallel row of said one or more first plant or plant population, line, cultivar, or variety; wherein said first or second plant or plant population, line, cultivar, or variety is a (genetically) male sterile plant or plant population, line, cultivar, or variety. Preferably the first and second plant are from the genus Triticum, preferably Triticum aestivum.

In an aspect, the invention relates to a method for sowing or planting for testing, evaluating, analyzing, or determining heterosis, comprising sowing seeds of one or more first plant or plant population, line, cultivar, or variety or planting plants of one or more first plant or plant population, line, cultivar, or variety in one or more parallel row; sowing seeds of one or more second plant or plant population, line, cultivar, or variety or planting plants of one or more second plant or plant population, line, cultivar, or variety in one or more parallel row flanking or flanked by said one or more parallel row of said one or more first plant or plant population, line, cultivar, or variety; wherein said first or second plant or plant population, line, cultivar, or variety is a (genetically) male sterile plant or plant population, line, cultivar, or variety. Preferably the first and second plant are from the genus Triticum, preferably Triticum aestivum.

In an aspect, the invention relates to a method for sowing or planting for plant hybrid test crossing or test cross evaluation (such as for evaluating (general and/or specific) combining ability or heterosis), comprising sowing seeds of one or more first plant or plant population, line, cultivar, or variety or planting plants of one or more first plant or plant population, line, cultivar, or variety in one or more parallel row; sowing seeds of one or more second plant or plant population, line, cultivar, or variety or planting plants of one or more second plant or plant population, line, cultivar, or variety in one or more parallel row flanking or flanked by said one or more parallel row of said one or more first plant or plant population, line, cultivar, or variety; wherein said first or second plant or plant population, line, cultivar, or variety is a (genetically) male sterile plant or plant population, line, cultivar, or variety. Preferably the first and second plant are from the genus Triticum, preferably Triticum aestivum.

In an aspect, the invention relates to a method for generating a hybrid plant, comprising sowing seeds of one or more first plant or plant population, line, cultivar, or variety or planting plants of one or more first plant or plant population, line, cultivar, or variety in one or more parallel row; sowing seeds of one or more second plant or plant population, line, cultivar, or variety or planting plants of one or more second plant or plant population, line, cultivar, or variety in one or more parallel row flanking or flanked by said one or more parallel row of said one or more first plant or plant population, line, cultivar, or variety; wherein said first or second plant or plant population, line, cultivar, or variety is a (genetically) male sterile plant or plant population, line, cultivar, or variety. Preferably the first and second plant are from the genus Triticum, preferably Triticum aestivum.

In an aspect, the invention relates to a method for testing, evaluating, analyzing, or determining (general and/or specific) combining ability, comprising sowing seeds of one or more first plant or plant population, line, cultivar, or variety or planting plants of one or more first plant or plant population, line, cultivar, or variety in one or more parallel row; sowing seeds of one or more second plant or plant population, line, cultivar, or variety or planting plants of one or more second plant or plant population, line, cultivar, or variety in one or more parallel row flanking or flanked by said one or more parallel row of said one or more first plant or plant population, line, cultivar, or variety; wherein said first or second plant or plant population, line, cultivar, or variety is a (genetically) male sterile plant or plant population, line, cultivar, or variety. Preferably the first and second plant are from the genus Triticum, preferably Triticum aestivum.

In an aspect, the invention relates to a method for testing, evaluating, analyzing, or determining heterosis, comprising sowing seeds of one or more first plant or plant population, line, cultivar, or variety or planting plants of one or more first plant or plant population, line, cultivar, or variety in one or more parallel row; sowing seeds of one or more second plant or plant population, line, cultivar, or variety or planting plants of one or more second plant or plant population, line, cultivar, or variety in one or more parallel row flanking or flanked by said one or more parallel row of said one or more first plant or plant population, line, cultivar, or variety; wherein said first or second plant or plant population, line, cultivar, or variety is a (genetically) male sterile plant or plant population, line, cultivar, or variety. Preferably the first and second plant are from the genus Triticum, preferably Triticum aestivum.

In an aspect, the invention relates to a method for plant hybrid test crossing or test cross evaluation (such as for evaluating (general and/or specific) combining ability or heterosis), comprising sowing seeds of one or more first plant or plant population, line, cultivar, or variety or planting plants of one or more first plant or plant population, line, cultivar, or variety in one or more parallel row; sowing seeds of one or more second plant or plant population, line, cultivar, or variety or planting plants of one or more second plant or plant population, line, cultivar, or variety in one or more parallel row flanking or flanked by said one or more parallel row of said one or more first plant or plant population, line, cultivar, or variety; wherein said first or second plant or plant population, line, cultivar, or variety is a (genetically) male sterile plant or plant population, line, cultivar, or variety. Preferably the first and second plant are from the genus Triticum, preferably Triticum aestivum.

In certain embodiments, the methods of the invention as described herein further comprise analyzing or determining one or more (agronomic, physiologic, or quality) characteristics or traits, such as for evaluating (general and/or specific) combining ability or heterosis or for evaluating hybrid test crossing or crosses.

It will be understood that according to the invention, if the first plant or plant population, line, cultivar, or variety is a (genetically) male sterile plant or plant population, line, cultivar, or variety, the second plant or plant population, line, cultivar, or variety is not a (genetically) male sterile plant or plant population, line, cultivar, or variety, and vice versa. Accordingly, if the first plant or plant population, line, cultivar, or variety is a (genetically) male sterile plant or plant population, line, cultivar, or variety, the second plant or plant population, line, cultivar, or variety is a (genetically) male fertile plant or plant population, line, cultivar, or variety, and vice versa.

In certain embodiments, the first plant or plant population, line, cultivar, or variety is a (genetically) male sterile plant or plant population, line, cultivar, or variety. In certain embodiments, the second plant or plant population, line, cultivar, or variety is a (genetically) male sterile plant or plant population, line, cultivar, or variety.

In certain embodiments, the first plant or plant population, line, cultivar, or variety is (from) an inbred line. In certain embodiments, the second plant or plant population, line, cultivar, or variety is (from) an inbred line. In certain embodiments, the first and second plant or plant population, line, cultivar, or variety is (from) an inbred line.

In certain embodiments, the first plant or plant population, line, cultivar, or variety is a tester plant or plant population, line, cultivar, or variety. In certain embodiments, the second plant or plant population, line, cultivar, or variety is tester plant or plant population, line, cultivar, or variety.

In certain embodiments, the first plant or plant population, line, cultivar, or variety is a (genetically) male sterile plant or plant population, line, cultivar, or variety selected from plants or plant populations, lines, cultivars, or varieties in a female pool of plants or plant populations, lines, cultivars, or varieties. In certain embodiments, the first plant or plant population, line, cultivar, or variety is a (genetically) male sterile plant or plant population, line, cultivar, or variety selected from plants or plant populations, lines, cultivars, or varieties in a male pool of plants or plant populations, lines, cultivars, or varieties. In certain embodiments, the second plant or plant population, line, cultivar, or variety is a (genetically) male sterile plant or plant population, line, cultivar, or variety selected from plants or plant populations, lines, cultivars, or varieties in a female pool of plants or plant populations, lines, cultivars, or varieties. In certain embodiments, the second plant or plant population, line, cultivar, or variety is a (genetically) male sterile plant or plant population, line, cultivar, or variety selected from plants or plant populations, lines, cultivars, or varieties in a male pool of plants or plant populations, lines, cultivars, or varieties. Advantageously, according to the present invention, male sterile testers from the male pool can still be used for production of test cross seed with lines relevant for the female pool where (robust) pollination ability is not a requirement. The fact that the tester and line can be grow for instance in rows spaced <20 cm apart ensures sufficient seed set even from poor pollinators.

In certain embodiments, the first plant or plant population, line, cultivar, or variety comprises an (alien) addition chromosome as described herein elsewhere. In certain embodiments, the first plant or plant population, line, cultivar, or variety comprises an (alien) addition chromosome comprising a genetic sterility restorer gene or genetic sterility restorer gene containing chromosomal fragment or locus. In certain embodiments, the first plant or plant population, line, cultivar, or variety comprises an (alien) addition chromosome comprising a genetic male sterility restorer gene or genetic male sterility restorer gene containing chromosomal fragment or locus. In certain embodiments, the first plant or plant population, line, cultivar, or variety comprises an (alien) addition chromosome comprising a male sterility restorer gene or a male sterility restorer gene containing chromosomal fragment or locus.

In certain embodiments, the first plant or plant population, line, cultivar, or variety comprises an (alien) addition chromosome comprising a genetic sterility restorer gene or genetic sterility restorer gene containing chromosomal fragment or locus and a selection marker. In certain embodiments, the first plant or plant population, line, cultivar, or variety comprises an (alien) addition chromosome comprising a genetic male sterility restorer gene or genetic male sterility restorer gene containing chromosomal fragment or locus and a selection marker. In certain embodiments, the first plant or plant population, line, cultivar, or variety comprises an (alien) addition chromosome comprising a male sterility restorer gene or male sterility restorer gene containing chromosomal fragment or locus and a selection marker.

In certain embodiments, the first plant or plant population, line, cultivar, or variety comprises an (alien) addition chromosome comprising a genetic sterility restorer gene or genetic sterility restorer gene containing chromosomal fragment or locus and a BLA gene or BLA encoding sequence. In certain embodiments, the first plant or plant population, line, cultivar, or variety comprises an (alien) addition chromosome comprising a genetic male sterility restorer gene or genetic male sterility restorer gene containing chromosomal fragment or locus and a BLA gene or BLA encoding sequence. In certain embodiments, the first plant or plant population, line, cultivar, or variety comprises an (alien) addition chromosome comprising a male sterility restorer gene or male sterility restorer gene containing chromosomal fragment or locus and a BLA gene or BLA encoding sequence.

In certain embodiments, the second plant or plant population, line, cultivar, or variety comprises an (alien) addition chromosome as described herein elsewhere. In certain embodiments, the second plant or plant population, line, cultivar, or variety comprises an (alien) addition chromosome comprising a genetic sterility restorer gene or genetic sterility restorer gene containing chromosomal fragment or locus. In certain embodiments, the second plant or plant population, line, cultivar, or variety comprises an (alien) addition chromosome comprising a genetic male sterility restorer gene or genetic male sterility restorer gene containing chromosomal fragment or locus. In certain embodiments, the second plant or plant population, line, cultivar, or variety comprises an (alien) addition chromosome comprising a male sterility restorer gene or male sterility restorer gene containing chromosomal fragment or locus.

In certain embodiments, the second plant or plant population, line, cultivar, or variety comprises an (alien) addition chromosome comprising a genetic sterility restorer gene or genetic sterility restorer gene containing chromosomal fragment or locus and a selection marker. In certain embodiments, the second plant or plant population, line, cultivar, or variety comprises an (alien) addition chromosome comprising a genetic male sterility restorer gene or genetic male sterility restorer gene containing chromosomal fragment or locus and a selection marker. In certain embodiments, the second plant or plant population, line, cultivar, or variety comprises an (alien) addition chromosome comprising a male sterility restorer gene or male sterility restorer gene containing chromosomal fragment or locus and a selection marker.

In certain embodiments, the second plant or plant population, line, cultivar, or variety comprises an (alien) addition chromosome comprising a genetic sterility restorer gene or genetic sterility restorer gene containing chromosomal fragment or locus and a BLA gene or BLA encoding sequence. In certain embodiments, the second plant or plant population, line, cultivar, or variety comprises an (alien) addition chromosome comprising a genetic male sterility restorer gene or genetic male sterility restorer gene containing chromosomal fragment or locus and a BLA gene or BLA encoding sequence. In certain embodiments, the second plant or plant population, line, cultivar, or variety comprises an (alien) addition chromosome comprising a male sterility restorer gene or male sterility restorer gene containing chromosomal fragment or locus and a BLA gene or BLA encoding sequence.

In certain embodiments, the tester plant or plant population, line, cultivar, or variety comprises an (alien) addition chromosome as described herein elsewhere. In certain embodiments, the tester plant or plant population, line, cultivar, or variety comprises an (alien) addition chromosome comprising a genetic sterility restorer gene or genetic sterility restorer gene containing chromosomal fragment or locus. In certain embodiments, the tester plant or plant population, line, cultivar, or variety comprises an (alien) addition chromosome comprising a genetic male sterility restorer gene or genetic male sterility restorer gene containing chromosomal fragment or locus. In certain embodiments, the tester plant or plant population, line, cultivar, or variety comprises an (alien) addition chromosome comprising a male sterility restorer gene or male sterility restorer gene containing chromosomal fragment or locus.

In certain embodiments, the tester plant or plant population, line, cultivar, or variety comprises an (alien) addition chromosome comprising a genetic sterility restorer gene or genetic sterility restorer gene containing chromosomal fragment or locus and a selection marker. In certain embodiments, the tester plant or plant population, line, cultivar, or variety comprises an (alien) addition chromosome comprising a genetic male sterility restorer gene or genetic male sterility restorer gene containing chromosomal fragment or locus and a selection marker. In certain embodiments, the tester plant or plant population, line, cultivar, or variety comprises an (alien) addition chromosome comprising a male sterility restorer gene or male sterility restorer gene containing chromosomal fragment or locus and a selection marker.

In certain embodiments, the tester plant or plant population, line, cultivar, or variety comprises an (alien) addition chromosome comprising a genetic sterility restorer gene or genetic sterility restorer gene containing chromosomal fragment or locus and a BLA gene or BLA encoding sequence. In certain embodiments, the tester plant or plant population, line, cultivar, or variety comprises an (alien) addition chromosome comprising a genetic male sterility restorer gene or genetic male sterility restorer gene containing chromosomal fragment or locus and a BLA gene or BLA encoding sequence. In certain embodiments, the tester plant or plant population, line, cultivar, or variety comprises an (alien) addition chromosome comprising a male sterility restorer gene or male sterility restorer gene containing chromosomal fragment or locus and a BLA gene or BLA encoding sequence.

In certain embodiments, the first plant or plant population, line, cultivar, or variety is a (genetically) (male) sterile plant or plant population, line, cultivar, or variety obtained by selecting seeds resulting from a cross between a (genetically) (male) sterile plant or plant population, line, cultivar, or variety and a (isogenic) plant or plant population, line, cultivar, or variety comprising an (alien) addition chromosome as described herein elsewhere, or obtained by selecting seeds resulting from self-fertilization of a plant or plant population, line, cultivar, or variety comprising an (alien) addition chromosome as described herein elsewhere.

In certain embodiments, the first plant or plant population, line, cultivar, or variety is a (genetically) (male) sterile plant or plant population, line, cultivar, or variety obtained by selecting seeds resulting from a cross between a (genetically) (male) sterile plant or plant population, line, cultivar, or variety and a (isogenic) plant or plant population, line, cultivar, or variety comprising an (alien) addition chromosome comprising a genetic sterility restorer gene or genetic sterility restorer gene containing chromosomal fragment or locus, or obtained by selecting seeds resulting from self-fertilization of a plant or plant population, line, cultivar, or variety comprising an (alien) addition chromosome comprising a genetic sterility restorer gene or genetic sterility restorer gene containing chromosomal fragment or locus.

In certain embodiments, the first plant or plant population, line, cultivar, or variety is a (genetically) (male) sterile plant or plant population, line, cultivar, or variety obtained by selecting seeds resulting from a cross between a (genetically) (male) sterile plant or plant population, line, cultivar, or variety and a (isogenic) plant or plant population, line, cultivar, or variety comprising an (alien) addition chromosome comprising a genetic male sterility restorer gene or genetic male sterility restorer gene containing chromosomal fragment or locus, or obtained by selecting seeds resulting from self-fertilization of a plant or plant population, line, cultivar, or variety comprising an (alien) addition chromosome comprising a genetic male sterility restorer gene or genetic male sterility restorer gene containing chromosomal fragment or locus.

In certain embodiments, the first plant or plant population, line, cultivar, or variety is a (genetically) (male) sterile plant or plant population, line, cultivar, or variety obtained by selecting seeds resulting from a cross between a (genetically) (male) sterile plant or plant population, line, cultivar, or variety and a (isogenic) plant or plant population, line, cultivar, or variety comprising an (alien) addition chromosome comprising a male sterility restorer gene or male sterility restorer gene containing chromosomal fragment or locus, or obtained by selecting seeds resulting from self-fertilization of a plant or plant population, line, cultivar, or variety comprising an (alien) addition chromosome comprising a male sterility restorer gene or male sterility restorer gene containing chromosomal fragment or locus.

In certain embodiments, the first plant or plant population, line, cultivar, or variety is a (genetically) (male) sterile plant or plant population, line, cultivar, or variety obtained by selecting seeds resulting from a cross between a (genetically) (male) sterile plant or plant population, line, cultivar, or variety and a (isogenic) plant or plant population, line, cultivar, or variety comprising an (alien) addition chromosome comprising a genetic sterility restorer gene or genetic sterility restorer gene containing chromosomal fragment or locus and a selection marker, or obtained by selecting seeds resulting from self-fertilization of a plant or plant population, line, cultivar, or variety comprising an (alien) addition chromosome comprising a genetic sterility restorer gene or genetic sterility restorer gene containing chromosomal fragment or locus and a selection marker.

In certain embodiments, the first plant or plant population, line, cultivar, or variety is a (genetically) (male) sterile plant or plant population, line, cultivar, or variety obtained by selecting seeds resulting from a cross between a (genetically) (male) sterile plant or plant population, line, cultivar, or variety and a (isogenic) plant or plant population, line, cultivar, or variety comprising an (alien) addition chromosome comprising a genetic male sterility restorer gene or genetic male sterility restorer gene containing chromosomal fragment or locus and a selection marker, or obtained by selecting seeds resulting from self-fertilization of a plant or plant population, line, cultivar, or variety comprising an (alien) addition chromosome comprising a genetic male sterility restorer gene or genetic male sterility restorer gene containing chromosomal fragment or locus and a selection marker.

In certain embodiments, the first plant or plant population, line, cultivar, or variety is a (genetically) (male) sterile plant or plant population, line, cultivar, or variety obtained by selecting seeds resulting from a cross between a (genetically) (male) sterile plant or plant population, line, cultivar, or variety and a (isogenic) plant or plant population, line, cultivar, or variety comprising an (alien) addition chromosome comprising a male sterility restorer gene or male sterility restorer gene containing chromosomal fragment or locus and a selection marker, or obtained by selecting seeds resulting from self-fertilization of a plant or plant population, line, cultivar, or variety comprising an (alien) addition chromosome comprising a male sterility restorer gene or male sterility restorer gene containing chromosomal fragment or locus and a selection marker.

In certain embodiments, the first plant or plant population, line, cultivar, or variety is a (genetically) (male) sterile plant or plant population, line, cultivar, or variety obtained by selecting seeds resulting from a cross between a (genetically) (male) sterile plant or plant population, line, cultivar, or variety and a (isogenic) plant or plant population, line, cultivar, or variety comprising an (alien) addition chromosome comprising a genetic sterility restorer gene or genetic sterility restorer gene containing chromosomal fragment or locus and a BLA gene or BLA encoding sequence, or obtained by selecting seeds resulting from self-fertilization of a plant or plant population, line, cultivar, or variety comprising an (alien) addition chromosome comprising a genetic sterility restorer gene or genetic sterility restorer gene containing chromosomal fragment or locus and a BLA gene or BLA encoding sequence.

In certain embodiments, the first plant or plant population, line, cultivar, or variety is a (genetically) (male) sterile plant or plant population, line, cultivar, or variety obtained by selecting seeds resulting from a cross between a (genetically) (male) sterile plant or plant population, line, cultivar, or variety and a (isogenic) plant or plant population, line, cultivar, or variety comprising an (alien) addition chromosome comprising a genetic male sterility restorer gene or genetic male sterility restorer gene containing chromosomal fragment or locus and a BLA gene or BLA encoding sequence, or obtained by selecting seeds resulting from self-fertilization of a plant or plant population, line, cultivar, or variety comprising an (alien) addition chromosome comprising a genetic male sterility restorer gene or genetic male sterility restorer gene containing chromosomal fragment or locus and a BLA gene or BLA encoding sequence.

In certain embodiments, the first plant or plant population, line, cultivar, or variety is a (genetically) (male) sterile plant or plant population, line, cultivar, or variety obtained by selecting seeds resulting from a cross between a (genetically) (male) sterile plant or plant population, line, cultivar, or variety and a (isogenic) plant or plant population, line, cultivar, or variety comprising an (alien) addition chromosome comprising a male sterility restorer gene or male sterility restorer gene containing chromosomal fragment or locus and a BLA gene or BLA encoding sequence, or obtained by selecting seeds resulting from self-fertilization of a plant or plant population, line, cultivar, or variety comprising an (alien) addition chromosome comprising a male sterility restorer gene or male sterility restorer gene containing chromosomal fragment or locus and a BLA gene or BLA encoding sequence.

In certain embodiments, the second plant or plant population, line, cultivar, or variety is a (genetically) (male) sterile plant or plant population, line, cultivar, or variety obtained by selecting seeds resulting from a cross between a (genetically) (male) sterile plant or plant population, line, cultivar, or variety and a (isogenic) plant or plant population, line, cultivar, or variety comprising an (alien) addition chromosome as described herein elsewhere, or obtained by selecting seeds resulting from self-fertilization of a plant or plant population, line, cultivar, or variety comprising an (alien) addition chromosome as described herein elsewhere.

In certain embodiments, the second plant or plant population, line, cultivar, or variety is a (genetically) (male) sterile plant or plant population, line, cultivar, or variety obtained by selecting seeds resulting from a cross between a (genetically) (male) sterile plant or plant population, line, cultivar, or variety and a (isogenic) plant or plant population, line, cultivar, or variety comprising an (alien) addition chromosome comprising a genetic sterility restorer gene or genetic sterility restorer gene containing chromosomal fragment or locus, or obtained by selecting seeds resulting from self-fertilization of a plant or plant population, line, cultivar, or variety comprising an (alien) addition chromosome comprising a genetic sterility restorer gene or genetic sterility restorer gene containing chromosomal fragment or locus.

In certain embodiments, the second plant or plant population, line, cultivar, or variety is a (genetically) (male) sterile plant or plant population, line, cultivar, or variety obtained by selecting seeds resulting from a cross between a (genetically) (male) sterile plant or plant population, line, cultivar, or variety and a (isogenic) plant or plant population, line, cultivar, or variety comprising an (alien) addition chromosome comprising a genetic male sterility restorer gene or genetic male sterility restorer gene containing chromosomal fragment or locus, or obtained by selecting seeds resulting from self-fertilization of a plant or plant population, line, cultivar, or variety comprising an (alien) addition chromosome comprising a genetic male sterility restorer gene or genetic male sterility restorer gene containing chromosomal fragment or locus.

In certain embodiments, the second plant or plant population, line, cultivar, or variety is a (genetically) (male) sterile plant or plant population, line, cultivar, or variety obtained by selecting seeds resulting from a cross between a (genetically) (male) sterile plant or plant population, line, cultivar, or variety and a (isogenic) plant or plant population, line, cultivar, or variety comprising an (alien) addition chromosome comprising a male sterility restorer gene or male sterility restorer gene containing chromosomal fragment or locus, or obtained by selecting seeds resulting from self-fertilization of a plant or plant population, line, cultivar, or variety comprising an (alien) addition chromosome comprising a male sterility restorer gene or male sterility restorer gene containing chromosomal fragment or locus.

In certain embodiments, the second plant or plant population, line, cultivar, or variety is a (genetically) (male) sterile plant or plant population, line, cultivar, or variety obtained by selecting seeds resulting from a cross between a (genetically) (male) sterile plant or plant population, line, cultivar, or variety and a (isogenic) plant or plant population, line, cultivar, or variety comprising an (alien) addition chromosome comprising a genetic sterility restorer gene or genetic sterility restorer gene containing chromosomal fragment or locus and a selection marker, or obtained by selecting seeds resulting from self-fertilization of a plant or plant population, line, cultivar, or variety comprising an (alien) addition chromosome comprising a genetic sterility restorer gene or genetic sterility restorer gene containing chromosomal fragment or locus and a selection marker.

In certain embodiments, the second plant or plant population, line, cultivar, or variety is a (genetically) (male) sterile plant or plant population, line, cultivar, or variety obtained by selecting seeds resulting from a cross between a (genetically) (male) sterile plant or plant population, line, cultivar, or variety and a (isogenic) plant or plant population, line, cultivar, or variety comprising an (alien) addition chromosome comprising a genetic male sterility restorer gene or genetic male sterility restorer gene containing chromosomal fragment or locus and a selection marker, or obtained by selecting seeds resulting from self-fertilization of a plant or plant population, line, cultivar, or variety comprising an (alien) addition chromosome comprising a genetic male sterility restorer gene or genetic male sterility restorer gene containing chromosomal fragment or locus and a selection marker.

In certain embodiments, the second plant or plant population, line, cultivar, or variety is a (genetically) (male) sterile plant or plant population, line, cultivar, or variety obtained by selecting seeds resulting from a cross between a (genetically) (male) sterile plant or plant population, line, cultivar, or variety and a (isogenic) plant or plant population, line, cultivar, or variety comprising an (alien) addition chromosome comprising a male sterility restorer gene or male sterility restorer gene containing chromosomal fragment or locus and a selection marker, or obtained by selecting seeds resulting from self-fertilization of a plant or plant population, line, cultivar, or variety comprising an (alien) addition chromosome comprising a male sterility restorer gene or male sterility restorer gene containing chromosomal fragment or locus and a selection marker.

In certain embodiments, the second plant or plant population, line, cultivar, or variety is a (genetically) (male) sterile plant or plant population, line, cultivar, or variety obtained by selecting seeds resulting from a cross between a (genetically) (male) sterile plant or plant population, line, cultivar, or variety and a (isogenic) plant or plant population, line, cultivar, or variety comprising an (alien) addition chromosome comprising a genetic sterility restorer gene or genetic sterility restorer gene containing chromosomal fragment or locus and a BLA gene or BLA encoding sequence, or obtained by selecting seeds resulting from self-fertilization of a plant or plant population, line, cultivar, or variety comprising an (alien) addition chromosome comprising a genetic sterility restorer gene or genetic sterility restorer gene containing chromosomal fragment or locus and a BLA gene or BLA encoding sequence.

In certain embodiments, the second plant or plant population, line, cultivar, or variety is a (genetically) (male) sterile plant or plant population, line, cultivar, or variety obtained by selecting seeds resulting from a cross between a (genetically) (male) sterile plant or plant population, line, cultivar, or variety and a (isogenic) plant or plant population, line, cultivar, or variety comprising an (alien) addition chromosome comprising a genetic male sterility restorer gene or genetic male sterility restorer gene containing chromosomal fragment or locus and a BLA gene or BLA encoding sequence, or obtained by selecting seeds resulting from self-fertilization of a plant or plant population, line, cultivar, or variety comprising an (alien) addition chromosome comprising a genetic male sterility restorer gene or genetic male sterility restorer gene containing chromosomal fragment or locus and a BLA gene or BLA encoding sequence.

In certain embodiments, the second plant or plant population, line, cultivar, or variety is a (genetically) (male) sterile plant or plant population, line, cultivar, or variety obtained by selecting seeds resulting from a cross between a (genetically) (male) sterile plant or plant population, line, cultivar, or variety and a (isogenic) plant or plant population, line, cultivar, or variety comprising an (alien) addition chromosome comprising a male sterility restorer gene or male sterility restorer gene containing chromosomal fragment or locus and a BLA gene or BLA encoding sequence, or obtained by selecting seeds resulting from self-fertilization of a plant or plant population, line, cultivar, or variety comprising an (alien) addition chromosome comprising a male sterility restorer gene or male sterility restorer gene containing chromosomal fragment or locus and a BLA gene or BLA encoding sequence.

In certain embodiments, the tester plant or plant population, line, cultivar, or variety is a (genetically) (male) sterile plant or plant population, line, cultivar, or variety obtained by selecting seeds resulting from a cross between a (genetically) (male) sterile plant or plant population, line, cultivar, or variety and a (isogenic) plant or plant population, line, cultivar, or variety comprising an (alien) addition chromosome as described herein elsewhere, or obtained by selecting seeds resulting from self-fertilization of a plant or plant population, line, cultivar, or variety comprising an (alien) addition chromosome as described herein elsewhere.

In certain embodiments, the tester plant or plant population, line, cultivar, or variety is a (genetically) (male) sterile plant or plant population, line, cultivar, or variety obtained by selecting seeds resulting from a cross between a (genetically) (male) sterile plant or plant population, line, cultivar, or variety and a (isogenic) plant or plant population, line, cultivar, or variety comprising an (alien) addition chromosome comprising a genetic sterility restorer gene or genetic sterility restorer gene containing chromosomal fragment or locus, or obtained by selecting seeds resulting from self-fertilization of a plant or plant population, line, cultivar, or variety comprising an (alien) addition chromosome comprising a genetic sterility restorer gene or genetic sterility restorer gene containing chromosomal fragment or locus.

In certain embodiments, the tester plant or plant population, line, cultivar, or variety is a (genetically) (male) sterile plant or plant population, line, cultivar, or variety obtained by selecting seeds resulting from a cross between a (genetically) (male) sterile plant or plant population, line, cultivar, or variety and a (isogenic) plant or plant population, line, cultivar, or variety comprising an (alien) addition chromosome comprising a genetic male sterility restorer gene or genetic male sterility restorer gene containing chromosomal fragment or locus, or obtained by selecting seeds resulting from self-fertilization of a plant or plant population, line, cultivar, or variety comprising an (alien) addition chromosome comprising a genetic male sterility restorer gene or genetic male sterility restorer gene containing chromosomal fragment or locus.

In certain embodiments, the tester plant or plant population, line, cultivar, or variety is a (genetically) (male) sterile plant or plant population, line, cultivar, or variety obtained by selecting seeds resulting from a cross between a (genetically) (male) sterile plant or plant population, line, cultivar, or variety and a (isogenic) plant or plant population, line, cultivar, or variety comprising an (alien) addition chromosome comprising a male sterility restorer gene or male sterility restorer gene containing chromosomal fragment or locus, or obtained by selecting seeds resulting from self-fertilization of a plant or plant population, line, cultivar, or variety comprising an (alien) addition chromosome comprising a male sterility restorer gene or male sterility restorer gene containing chromosomal fragment or locus.

In certain embodiments, the tester plant or plant population, line, cultivar, or variety is a (genetically) (male) sterile plant or plant population, line, cultivar, or variety obtained by selecting seeds resulting from a cross between a (genetically) (male) sterile plant or plant population, line, cultivar, or variety and a (isogenic) plant or plant population, line, cultivar, or variety comprising an (alien) addition chromosome comprising a genetic sterility restorer gene or genetic sterility restorer gene containing chromosomal fragment or locus and a selection marker, or obtained by selecting seeds resulting from self-fertilization of a plant or plant population, line, cultivar, or variety comprising an (alien) addition chromosome comprising a genetic sterility restorer gene or genetic sterility restorer gene containing chromosomal fragment or locus and a selection marker.

In certain embodiments, the tester plant or plant population, line, cultivar, or variety is a (genetically) (male) sterile plant or plant population, line, cultivar, or variety obtained by selecting seeds resulting from a cross between a (genetically) (male) sterile plant or plant population, line, cultivar, or variety and a (isogenic) plant or plant population, line, cultivar, or variety comprising an (alien) addition chromosome comprising a genetic male sterility restorer gene or genetic male sterility restorer gene containing chromosomal fragment or locus and a selection marker, or obtained by selecting seeds resulting from self-fertilization of a plant or plant population, line, cultivar, or variety comprising an (alien) addition chromosome comprising a genetic male sterility restorer gene or genetic male sterility restorer gene containing chromosomal fragment or locus and a selection marker.

In certain embodiments, the tester plant or plant population, line, cultivar, or variety is a (genetically) (male) sterile plant or plant population, line, cultivar, or variety obtained by selecting seeds resulting from a cross between a (genetically) (male) sterile plant or plant population, line, cultivar, or variety and a (isogenic) plant or plant population, line, cultivar, or variety comprising an (alien) addition chromosome comprising a male sterility restorer gene or male sterility restorer gene containing chromosomal fragment or locus and a selection marker, or obtained by selecting seeds resulting from self-fertilization of a plant or plant population, line, cultivar, or variety comprising an (alien) addition chromosome comprising a male sterility restorer gene or male sterility restorer gene containing chromosomal fragment or locus and a selection marker.

In certain embodiments, the tester plant or plant population, line, cultivar, or variety is a (genetically) (male) sterile plant or plant population, line, cultivar, or variety obtained by selecting seeds resulting from a cross between a (genetically) (male) sterile plant or plant population, line, cultivar, or variety and a (isogenic) plant or plant population, line, cultivar, or variety comprising an (alien) addition chromosome comprising a genetic sterility restorer gene or genetic sterility restorer gene containing chromosomal fragment or locus and a BLA gene or BLA encoding sequence, or obtained by selecting seeds resulting from self-fertilization of a plant or plant population, line, cultivar, or variety comprising an (alien) addition chromosome comprising a genetic sterility restorer gene or genetic sterility restorer gene containing chromosomal fragment or locus and a BLA gene or BLA encoding sequence.

In certain embodiments, the tester plant or plant population, line, cultivar, or variety is a (genetically) (male) sterile plant or plant population, line, cultivar, or variety obtained by selecting seeds resulting from a cross between a (genetically) (male) sterile plant or plant population, line, cultivar, or variety and a (isogenic) plant or plant population, line, cultivar, or variety comprising an (alien) addition chromosome comprising a genetic male sterility restorer gene or genetic male sterility restorer gene containing chromosomal fragment or locus and a BLA gene or BLA encoding sequence, or obtained by selecting seeds resulting from self-fertilization of a plant or plant population, line, cultivar, or variety comprising an (alien) addition chromosome comprising a genetic male sterility restorer gene or genetic male sterility restorer gene containing chromosomal fragment or locus and a BLA gene or BLA encoding sequence.

In certain embodiments, the tester plant or plant population, line, cultivar, or variety is a (genetically) (male) sterile plant or plant population, line, cultivar, or variety obtained by selecting seeds resulting from a cross between a (genetically) (male) sterile plant or plant population, line, cultivar, or variety and a (isogenic) plant or plant population, line, cultivar, or variety comprising an (alien) addition chromosome comprising a male sterility restorer gene or male sterility restorer gene containing chromosomal fragment or locus and a BLA gene or BLA encoding sequence, or obtained by selecting seeds resulting from self-fertilization of a plant or plant population, line, cultivar, or variety comprising an (alien) addition chromosome comprising a male sterility restorer gene or male sterility restorer gene containing chromosomal fragment or locus and a BLA gene or BLA encoding sequence.

The skilled person will understand that preferably according to the invention as described herein, only one of both (parent) plants/populations (i.e. the first or second plant/population) comprises the addition chromosome, if at all present. The other (parent) plant/population preferably is sterile, preferably male sterile or genetically sterile, more preferably genetically male sterile, as described herein elsewhere, e.g. having an (homozygous) ms mutation, such as an (homozygous) ms1 mutation, such as deletion, knockdown, or knockout.

The skilled person will understand that preferably the (parent) plants/populations having the (alien) addition chromosome or having at least the restorer gene (and selection marker), preferably comprised in an (alien) addition chromosome also comprise the genetic event leading to sterility, which however is (phenotypically) suppressed or negated by the presence of the restorer gene. Accordingly, in certain preferred embodiments the (parent) plants/populations having the (alien) addition chromosome or having at least the restorer gene (and selection marker), preferably comprised in an (alien) addition chromosome, comprise a mutation, as referred to herein elsewhere, in an ms gene, such as preferably ms1, preferably homozygous, or in all alleles.

The skilled person will understand that selection of plants or plant populations can be performed genotypically or phenotypically, as is known in the art, such as for instance by selection based on the selection marker as described herein elsewhere, such as for instance the BLA gene, which allows selection based on seed colour (i.e. blue seeds comprise the BLA gene and the restorer gene and hence are (genetically) (male) fertile, and non-blue seeds or white seeds lack the BLA gene and the restorer gene and hence are (genetically) (male) sterile (when resulting from crosses involving (genetic) (male) sterile plants and (isogenic) plants comprising the same (genetic) (male) sterility factors/genes and in addition a restorer gene and a BLA gene)). Accordingly, in certain embodiments, selection involves selecting blue seeds (if (genetic) (male) fertile plants are to be selected). In certain embodiments, selection involves selecting non-blue or white seeds (if (genetic) (male) sterile plants are to be selected).

Preferably, the (genetic) (male) sterile plant as used herein is a genetic (male) sterile plant or a (genetic) male sterile plant, preferably a genetically male sterile plant. Preferably, such plants have a mutated ms gene, as described herein elsewhere, preferably a mutated ms1 gene, as described herein elsewhere, such as a ms(1) gene deletion, knockout, or knockdown.

Preferably, as used herein, the sterile plant or plant population, line, cultivar, or variety, is a genetically male sterile plant or plant population, line, cultivar, or variety.

In certain embodiments, the first plant or plant population, line, cultivar, or variety is from the family of Poaceae. In certain embodiments, the second plant or plant population, line, cultivar, or variety is from the family of Poaceae. In certain embodiments, the first and second plant or plant population, line, cultivar, or variety is from the family of Poaceae. In certain embodiments, the first plant or plant population, line, cultivar, or variety is from the subfamily of Pooideae. In certain embodiments, the second plant or plant population, line, cultivar, or variety is from the subfamily of Pooideae. In certain embodiments, the first and second plant or plant population, line, cultivar, or variety is from the subfamily of Pooideae. In certain embodiments, the first plant or plant population, line, cultivar, or variety is from the tribe of Triticeae. In certain embodiments, the second plant or plant population, line, cultivar, or variety is from the tribe of Triticeae. In certain embodiments, the first and second plant or plant population, line, cultivar, or variety is from the tribe of Triticeae. In certain embodiments, the first plant or plant population, line, cultivar, or variety is from the genus of Triticum. In certain embodiments, the second plant or plant population, line, cultivar, or variety is from the genus of Triticum. In certain embodiments, the first and second plant or plant population, line, cultivar, or genus is from the family of Triticum. In certain embodiments, the first plant or plant population, line, cultivar, or variety is from the species Triticum aestivum. In certain embodiments, the second plant or plant population, line, cultivar, or variety is from the species Triticum aestivum. In certain embodiments, the first and second plant or plant population, line, cultivar, or genus is from the species Triticum aestivum.

In an aspect, the invention relates to the use of a planting scheme of the invention as described herein elsewhere (in a method) for testing, analyzing, evaluating, or determining heterosis or general and/or specific combining ability of or in plants or (in a method) for plant hybrid test crossing. Preferably the plants are from the genus Triticum, preferably Triticum aestivum.

In an aspect, the invention relates to the use of a (genetically) male sterile plant or plant population, line, cultivar, or variety (in a method) for testing, analyzing, evaluating, or determining heterosis or general and/or specific combining ability of or in plants or (in a method) for plant hybrid test crossing. Preferably the plants are from the genus Triticum, preferably Triticum aestivum.

In an aspect, the invention relates to a hybrid plant obtained or obtainable by the methods of the invention as described herein, or a plant part, such as a seed.

Planting Schemes

In certain embodiments, planting schemes (or sowing schemes) as described herein further below are employed according to the methods of the invention as described herein. Such planting schemes may include one or more of planting or sowing patterns, dimensions, density, etc. as further described below.

In certain embodiments, planting schemes, combine any one or more of the aspects and sub-aspects detailed hereafter.

In certain embodiments, planting schemes combine any one or more of aspects {A}, {B}, {C}, {D}, {E}, {F}, {G}, {A+B}, {A+C}, {A+D}, {A+E}, {A+F}, {A+G}, {B+C}, {B+D}, {B+E}, {B+F}, {B+G}, {C+D}, {C+E}, {C+F}, {C+G}, {D+E}, {D+F}, {D+G}, {E+F}, {E+G}, {F+G}, {A+B+C}, {A+B+D}, {A+B+E}, {A+B+F}, {A+B+G}, {A+C+D}, {A+C+E}, {A+C+F}, {A+C+G}, {A+D+E}, {A+D+F}, {A+D+G}, {A+E+F}, {A+E+G}, {A+F+G}, {B+C+D}, {B+C+E}, {B+C+F}, {B+C+G}, {B+D+E}, {B+D+F}, {B+D+G}, {B+E+F}, {B+E+G}, {B+F+G}, {C+D+E}, {C+D+F}, {C+D+G}, {C+E+F}, {C+E+G}, {C+F+G}, {D+E+F}, {D+E+G}, {D+F+G}, {E+F+G}, {A+B+C+D}, {A+B+C+E}, {A+B+C+F}, {A+B+C+G}, {A+B+D+E}, {A+B+D+F}, {A+B+D+G}, {A+B+E+F}, {A+B+E+G}, {A+B+F+G}, {A+C+D+E}, {A+C+D+F}, {A+C+D+G}, {A+C+E+F}, {A+C+E+G}, {A+C+F+G}, {A+D+E+F}, {A+D+E+G}, {A+D+F+G}, {A+E+F+G}, {B+C+D+E}, {B+C+D+F}, {B+C+D+G}, {B+C+E+F}, {B+C+E+G}, {B+C+F+G}, {B+D+E+F}, {B+D+E+G}, {B+D+F+G}, {B+E+F+G}, {C+D+E+F}, {C+D+E+G}, {C+D+F+G}, {C+E+F+G}, {D+E+F+G}, {A+B+C+D+E}, {A+B+C+D+F}, {A+B+C+D+G}, {A+B+C+E+F}, {A+B+C+E+G}, {A+B+C+F+G}, {A+B+D+E+F}, {A+B+D+E+G}, {A+B+D+F+G}, {A+B+E+F+G}, {A+C+D+E+F}, {A+C+D+E+G}, {A+C+D+F+G}, {A+C+E+F+G}, {A+D+E+F+G}, {B+C+D+E+F}, {B+C+D+E+G}, {B+C+D+F+G}, {B+C+E+F+G}, {B+D+E+F+G}, {C+D+E+F+G}, {A+B+C+D+E+F}, {A+B+C+D+E+G}, {A+B+C+D+F+G}, {A+B+C+E+F+G}, {A+B+D+E+F+G}, {A+C+D+E+F+G}, {B+C+D+E+F+G}, {A+B+C+D+E+F+G}.

Aspect A
A1 In certain embodiments, plants are planted, or seeds thereof are sown or have been sown in one or more rows. In certain embodiments, plants of a first plant or plant population, line, cultivar, or variety are planted or seeds thereof are sown or have been sown in one or more rows. In certain embodiments, plants of a second plant or plant population, line, cultivar, or variety are planted or seeds thereof are sown or have been sown in one or more rows. In certain embodiments, plants of a first and a second plant or plant population, line, cultivar, or variety are planted or seeds thereof are sown or have been sown in one or more rows. In certain embodiments, plants of a first plant or plant population, line, cultivar, or variety are planted or seeds thereof are sown or have been sown in one or more rows flanking or flanked (i.e. adjacent) by one or more rows of plants or seeds of a second plant or plant population, line, cultivar, or variety. In certain embodiments, plants of a first plant or plant population, line, cultivar, or variety are planted or seeds thereof are sown or have been sown in one or more rows flanking or flanked by plants or seeds of a second plant or plant population, line, cultivar, or variety. In certain embodiments, plants of a first plant or plant population, line, cultivar, or variety are planted or seeds thereof are sown or have been sown flanking or flanked by one or more rows of plants or seeds of a second plant or plant population, line, cultivar, or variety.
A2 In certain embodiments, plants are planted, or seeds thereof are sown or have been sown in one or more parallel rows. In certain embodiments, plants of a first plant or plant population, line, cultivar, or variety are planted or seeds thereof are sown or have been sown in one or more parallel rows. In certain embodiments, plants of a second plant or plant population, line, cultivar, or variety are planted or seeds thereof are sown or have been sown in one or more parallel rows. In certain embodiments, plants of a first and a second plant or plant population, line, cultivar, or variety are planted or seeds thereof are sown or have been sown in one or more parallel rows. In certain embodiments, plants of a first plant or plant population, line, cultivar, or variety are planted or seeds thereof are sown or have been sown in one or more parallel rows flanking or flanked by one or more parallel rows of plants or seeds of a second plant or plant population, line, cultivar, or variety. In certain embodiments, plants of a first plant or plant population, line, cultivar, or variety are planted or seeds thereof are sown or have been sown in one or more parallel rows flanking or flanked by one or more rows of plants or seeds of a second plant or plant population, line, cultivar, or variety. In certain embodiments, plants of a first plant or plant population, line, cultivar, or variety are planted or seeds thereof are sown or have been sown in one or more rows flanking or flanked by one or more parallel rows of plants or seeds of a second plant or plant population, line, cultivar, or variety.
A3 In certain embodiments, the number of rows is (at most) 10, such as ranging from 2 to 10. In certain embodiments, the number of rows is (at most) 9, such as ranging from 2 to 9. In certain embodiments, the number of rows is (at most) 8, such as ranging from 2 to 8. In certain embodiments, the number of rows is (at most) 7, such as ranging from 2 to 7. In certain embodiments, the number of rows is (at most) 6, such as ranging from 2 to 6. In certain embodiments, the number of rows is (at most) 5, such as ranging from 2 to 5. In certain embodiments, the number of rows is (at most) 4, such as ranging from 2 to 4. In certain embodiments, the number of rows is (at most) 3, such as ranging from 2 to 3. In certain embodiments, the number of rows is (at most) 2. In certain embodiments, the number of rows is 1.
A4 In certain embodiments, the number of parallel rows is (at most) 10, such as ranging from 2 to 10. In certain embodiments, the number of parallel rows is (at most) 9, such as ranging from 2 to 9. In certain embodiments, the number of parallel rows is (at most) 8, such as ranging from 2 to 8. In certain embodiments, the number of parallel rows is (at most) 7, such as ranging from 2 to 7. In certain embodiments, the number of parallel rows is (at most) 6, such as ranging from 2 to 6. In certain embodiments, the number of parallel rows is (at most) 5, such as ranging from 2 to 5. In certain embodiments, the number of parallel rows is (at most) 4, such as ranging from 2 to 4. In certain embodiments, the number of parallel rows is (at most) 3, such as ranging from 2 to 3. In certain embodiments, the number of parallel rows is (at most) 2. In certain embodiments, the number of parallel rows is 1.
A5 In certain embodiments, the number of rows of the first plant or plant population, line, cultivar, or variety is (at most) 10, such as ranging from 2 to 10. In certain embodiments, the number of rows of the first plant or plant population, line, cultivar, or variety is (at most) 9, such as ranging from 2 to 9. In certain embodiments, the number of rows of the first plant or plant population, line, cultivar, or variety is (at most) 8, such as ranging from 2 to 8. In certain embodiments, the number of rows of the first plant or plant population, line, cultivar, or variety is (at most) 7, such as ranging from 2 to 7. In certain embodiments, the number of rows of the first plant or plant population, line, cultivar, or variety is (at most) 6, such as ranging from 2 to 6. In certain embodiments, the number of rows of the first plant or plant population, line, cultivar, or variety is (at most) 5, such as ranging from 2 to 5. In certain embodiments, the number of rows of the first plant or plant population, line, cultivar, or variety is (at most) 4, such as ranging from 2 to 4. In certain embodiments, the number of rows of the first plant or plant population, line, cultivar, or variety is (at most) 3, such as ranging from 2 to 3. In certain embodiments, the number of rows of the first plant or plant population, line, cultivar, or variety is (at most) 2. In certain embodiments, the number of rows of the first plant or plant population, line, cultivar, or variety is 1.
A6 In certain embodiments, the number of parallel rows of the first plant or plant population, line, cultivar, or variety is (at most) 10, such as ranging from 2 to 10. In certain embodiments, the number of parallel rows of the first plant or plant population, line, cultivar, or variety is (at most) 9, such as ranging from 2 to 9. In certain embodiments, the number of parallel rows of the first plant or plant population, line, cultivar, or variety is (at most) 8, such as ranging from 2 to 8. In certain embodiments, the number of parallel rows of the first plant or plant population, line, cultivar, or variety is (at most) 7, such as ranging from 2 to 7. In certain embodiments, the number of parallel rows of the first plant or plant population, line, cultivar, or variety is (at most) 6, such as ranging from 2 to 6. In certain embodiments, the number of parallel rows of the first plant or plant population, line, cultivar, or variety is (at most) 5, such as ranging from 2 to 5. In certain embodiments, the number of parallel rows of the first plant or plant population, line, cultivar, or variety is (at most) 4, such as ranging from 2 to 4. In certain embodiments, the number of parallel rows of the first plant or plant population, line, cultivar, or variety is (at most) 3, such as ranging from 2 to 3. In certain embodiments, the number of parallel rows of the first plant or plant population, line, cultivar, or variety is (at most) 2. In certain embodiments, the number of parallel rows of the first plant or plant population, line, cultivar, or variety is 1.
A7 In certain embodiments, the number of rows of the second plant or plant population, line, cultivar, or variety is (at most) 10, such as ranging from 2 to 10. In certain embodiments, the number of rows of the second plant or plant population, line, cultivar, or variety is (at most) 9, such as ranging from 2 to 9. In certain embodiments, the number of rows of the second plant or plant population, line, cultivar, or variety is (at most) 8, such as ranging from 2 to 8. In certain embodiments, the number of rows of the second plant or plant population, line, cultivar, or variety is (at most) 7, such as ranging from 2 to 7. In certain embodiments, the number of rows of the second plant or plant population, line, cultivar, or variety is (at most) 6, such as ranging from 2 to 6. In certain embodiments, the number of rows of the second plant or plant population, line, cultivar, or variety is (at most) 5, such as ranging from 2 to 5. In certain embodiments, the number of rows of the second plant or plant population, line, cultivar, or variety is (at most) 4, such as ranging from 2 to 4. In certain embodiments, the number of rows of the second plant or plant population, line, cultivar, or variety is (at most) 3, such as ranging from 2 to 3. In certain embodiments, the number of rows of the second plant or plant population, line, cultivar, or variety is (at most) 2. In certain embodiments, the number of rows of the second plant or plant population, line, cultivar, or variety is 1.
A8 In certain embodiments, the number of parallel rows of the second plant or plant population, line, cultivar, or variety is (at most) 10, such as ranging from 2 to 10. In certain embodiments, the number of parallel rows of the second plant or plant population, line, cultivar, or variety is (at most) 9, such as ranging from 2 to 9. In certain embodiments, the number of parallel rows of the second plant or plant population, line, cultivar, or variety is (at most) 8, such as ranging from 2 to 8. In certain embodiments, the number of parallel rows of the second plant or plant population, line, cultivar, or variety is (at most) 7, such as ranging from 2 to 7. In certain embodiments, the number of parallel rows of the second plant or plant population, line, cultivar, or variety is (at most) 6, such as ranging from 2 to 6. In certain embodiments, the number of parallel rows of the second plant or plant population, line, cultivar, or variety is (at most) 5, such as ranging from 2 to 5. In certain embodiments, the number of parallel rows of the second plant or plant population, line, cultivar, or variety is (at most) 4, such as ranging from 2 to 4. In certain embodiments, the number of parallel rows of the second plant or plant population, line, cultivar, or variety is (at most) 3, such as ranging from 2 to 3. In certain embodiments, the number of parallel rows of the second plant or plant population, line, cultivar, or variety is (at most) 2. In certain embodiments, the number of parallel rows of the second plant or plant population, line, cultivar, or variety is 1.
A9 In certain embodiments, the number of rows of the first and second plant or plant population, line, cultivar, or variety is (at most) 10, such as ranging from 2 to 10. In certain embodiments, the number of rows of the first and second plant or plant population, line, cultivar, or variety is (at most) 9, such as ranging from 2 to 9. In certain embodiments, the number of rows of the first and second plant or plant population, line, cultivar, or variety is (at most) 8, such as ranging from 2 to 8. In certain embodiments, the number of rows of the first and second plant or plant population, line, cultivar, or variety is (at most) 7, such as ranging from 2 to 7. In certain embodiments, the number of rows of the first and second plant or plant population, line, cultivar, or variety is (at most) 6, such as ranging from 2 to 6. In certain embodiments, the number of rows of the first and second plant or plant population, line, cultivar, or variety is (at most) 5, such as ranging from 2 to 5. In certain embodiments, the number of rows of the first and second plant or plant population, line, cultivar, or variety is (at most) 4, such as ranging from 2 to 4. In certain embodiments, the number of rows of the first and second plant or plant population, line, cultivar, or variety is (at most) 3, such as ranging from 2 to 3. In certain embodiments, the number of rows of the first and second plant or plant population, line, cultivar, or variety is (at most) 2. In certain embodiments, the number of rows of the first and second plant or plant population, line, cultivar, or variety is 1.
A10 In certain embodiments, the number of parallel rows of the first and second plant or plant population, line, cultivar, or variety is (at most) 10, such as ranging from 2 to 10. In certain embodiments, the number of parallel rows of the first and second plant or plant population, line, cultivar, or variety is (at most) 9, such as ranging from 2 to 9. In certain embodiments, the number of parallel rows of the first and second plant or plant population, line, cultivar, or variety is (at most) 8, such as ranging from 2 to 8. In certain embodiments, the number of parallel rows of the first and second plant or plant population, line, cultivar, or variety is (at most) 7, such as ranging from 2 to 7. In certain embodiments, the number of parallel rows of the first and second plant or plant population, line, cultivar, or variety is (at most) 6, such as ranging from 2 to 6. In certain embodiments, the number of parallel rows of the first and second plant or plant population, line, cultivar, or variety is (at most) 5, such as ranging from 2 to 5. In certain embodiments, the number of parallel rows of the first and second plant or plant population, line, cultivar, or variety is (at most) 4, such as ranging from 2 to 4. In certain embodiments, the number of parallel rows of the first and second plant or plant population, line, cultivar, or variety is (at most) 3, such as ranging from 2 to 3. In certain embodiments, the number of parallel rows of the first and second plant or plant population, line, cultivar, or variety is (at most) 2. In certain embodiments, the number of parallel rows of the first and second plant or plant population, line, cultivar, or variety is 1.

In certain embodiments one or more row of a first plant or plant population, line, cultivar, or variety may be surrounded by one or more rows of a second plant, or plant population, line, cultivar, or variety. In certain embodiments an area (which may or may not be organized in rows) of a first plant or plant population, line, cultivar, or variety may be surrounded (on all or some sides, such as flanked on opposing sides) by one or more rows of a second plant, or plant population, line, cultivar, or variety.

Aspect B
B1 In certain embodiments, each row is at most 1 m spaced apart, preferably ranging from 10 cm to 1 m, such as from 10 cm to 90 cm, 10 cm to 80 cm, 10 cm to 70 cm, 10 cm to 50 cm, 10 cm to 40 cm 10 cm to 30 cm, or 10 cm to 20 cm. In certain embodiments, each parallel row is at most 1 m spaced apart, preferably ranging from 10 cm to 1 m, such as from 10 cm to 90 cm, 10 cm to 80 cm, 10 cm to 70 cm, 10 cm to 50 cm, 10 cm to 40 cm, 10 cm to 30 cm, or 10 cm to 20 cm.
B2 In certain embodiments, each row of the first plant or plant population, line, cultivar, or variety is at most 1 m spaced apart, preferably ranging from 10 cm to 1 m, such as from 10 cm to 90 cm, 10 cm to 80 cm, 10 cm to 70 cm, 10 cm to 50 cm, 10 cm to 40 cm, 10 cm to 30 cm, or 10 cm to 20 cm. In certain embodiments, each parallel row of the first plant or plant population, line, cultivar, or variety is at most 1 m spaced apart, preferably ranging from 10 cm to 1 m, such as from 10 cm to 90 cm, 10 cm to 80 cm, 10 cm to 70 cm, 10 cm to 50 cm, 10 cm to 40 cm, 10 cm to 30 cm, or 10 cm to 20 cm.
B3 In certain embodiments, each row of the second plant or plant population, line, cultivar, or variety is at most 1 m spaced apart, preferably ranging from 10 cm to 1 m, such as from 10 cm to 90 cm, 10 cm to 80 cm, 10 cm to 70 cm, 10 cm to 50 cm, 10 cm to 40 cm 10 cm to 30 cm, or 10 cm to 20 cm. In certain embodiments, each parallel row of the second plant or plant population, line, cultivar, or variety is at most 1 m spaced apart, preferably ranging from 10 cm to 1 m, such as from 10 cm to 90 cm, 10 cm to 80 cm, 10 cm to 70 cm, 10 cm to 50 cm, 10 cm to 40 cm, 10 cm to 30 cm, or 10 cm to 20 cm.
B4 In certain embodiments, each row of the first and second plant or plant population, line, cultivar, or variety is at most 1 m spaced apart, preferably ranging from 10 cm to 1 m, such as from 10 cm to 90 cm, 10 cm to 80 cm, 10 cm to 70 cm, 10 cm to 50 cm, 10 cm to 40 cm 10 cm to 30 cm, or 10 cm to 20 cm. In certain embodiments, each parallel row of the first and second plant or plant population, line, cultivar, or variety is at most 1 m spaced apart, preferably ranging from 10 cm to 1 m, such as from 10 cm to 90 cm, 10 cm to 80 cm, 10 cm to 70 cm, 10 cm to 50 cm, 10 cm to 40 cm, 10 cm to 30 cm, or 10 cm to 20 cm.

It will be understood that (parallel) rows within a specific plant or plant population, line, cultivar, or variety may have the same or a different spacing and that rows between different plants or plant populations, lines, cultivars, or varieties may have the same or a different spacing. By means of example and without limitations, two parallel rows of plant line A may be spaced apart 0.5 m, two parallel rows of pant line B may be spaced apart 0.75 m and the adjacent row(s) of plant line A and plant line B may be spaced apart 1 m.

It will be understood that in the context of the present invention, the term “spaced apart” refers to the (average or mean) distance of adjacent rows.

Aspect C
C1 In certain embodiments, each row is at most 15 m long, preferably ranging from 1 m to 15 m, such as from 1 m to 14 m, 1 m to 13 m, 1 m to 12 m, 1 m to 11 m, 1 m to 10 m, 2 m to 15 m, such as from 12 m to 14 m, 2 m to 13 m, 2 m to 12 m, 2 m to 11 m, 2 m to 10 m, 3 m to 15 m, such as from 3 m to 14 m, 3 m to 13 m, 3 m to 12 m, 3 m to 11 m, 3 m to 10 m, 4 m to 15 m, such as from 4 m to 14 m, 4 m to 13 m, 4 m to 12 m, 4 m to 11 m, 4 m to 10 m, 5 m to 15 m, such as from 5 m to 14 m, 5 m to 13 m, 5 m to 12 m, 5 m to 11 m, 5 m to 10 m, such as (about) 1, 2, 3, 4, 5, 6, 7, 8, 9, 10, 11, 12, 13, 14, or 15 m. In certain embodiments, each parallel row is at most 15 m long, preferably ranging from 1 m to 15 m, such as from 1 m to 14 m, 1 m to 13 m, 1 m to 12 m, 1 m to 11 m, 1 m to 10 m, 2 m to 15 m, such as from 12 m to 14 m, 2 m to 13 m, 2 m to 12 m, 2 m to 11 m, 2 m to 10 m, 3 m to 15 m, such as from 3 m to 14 m, 3 m to 13 m, 3 m to 12 m, 3 m to 11 m, 3 m to 10 m, 4 m to 15 m, such as from 4 m to 14 m, 4 m to 13 m, 4 m to 12 m, 4 m to 11 m, 4 m to 10 m, 5 m to 15 m, such as from 5 m to 14 m, 5 m to 13 m, 5 m to 12 m, 5 m to 11 m, 5 m to 10 m, such as (about) 1, 2, 3, 4, 5, 6, 7, 8, 9, 10, 11, 12, 13, 14, or 15 m.
C2 In certain embodiments, each row of the first plant or plant population, line, cultivar, or variety is at most 15 m long, preferably ranging from 1 m to 15 m, such as from 1 m to 14 m, 1 m to 13 m, 1 m to 12 m, 1 m to 11 m, 1 m to 10 m, 2 m to 15 m, such as from 12 m to 14 m, 2 m to 13 m, 2 m to 12 m, 2 m to 11 m, 2 m to 10 m, 3 m to 15 m, such as from 3 m to 14 m, 3 m to 13 m, 3 m to 12 m, 3 m to 11 m, 3 m to 10 m, 4 m to 15 m, such as from 4 m to 14 m, 4 m to 13 m, 4 m to 12 m, 4 m to 11 m, 4 m to 10 m, 5 m to 15 m, such as from 5 m to 14 m, 5 m to 13 m, 5 m to 12 m, 5 m to 11 m, 5 m to 10 m, such as (about) 1, 2, 3, 4, 5, 6, 7, 8, 9, 10, 11, 12, 13, 14, or 15 m. In certain embodiments, each parallel row of the first plant or plant population, line, cultivar, or variety is at most 15 m long, preferably ranging from 1 m to 15 m, such as from 1 m to 14 m, 1 m to 13 m, 1 m to 12 m, 1 m to 11 m, 1 m to 10 m, 2 m to 15 m, such as from 12 m to 14 m, 2 m to 13 m, 2 m to 12 m, 2 m to 11 m, 2 m to 10 m, 3 m to 15 m, such as from 3 m to 14 m, 3 m to 13 m, 3 m to 12 m, 3 m to 11 m, 3 m to 10 m, 4 m to 15 m, such as from 4 m to 14 m, 4 m to 13 m, 4 m to 12 m, 4 m to 11 m, 4 m to 10 m, 5 m to 15 m, such as from 5 m to 14 m, 5 m to 13 m, 5 m to 12 m, 5 m to 11 m, 5 m to 10 m, such as (about) 1, 2, 3, 4, 5, 6, 7, 8, 9, 10, 11, 12, 13, 14, or 15 m.
C3 In certain embodiments, each row of the second plant or plant population, line, cultivar, or variety is at most 15 m long, preferably ranging from 1 m to 15 m, such as from 1 m to 14 m, 1 m to 13 m, 1 m to 12 m, 1 m to 11 m, 1 m to 10 m, 2 m to 15 m, such as from 12 m to 14 m, 2 m to 13 m, 2 m to 12 m, 2 m to 11 m, 2 m to 10 m, 3 m to 15 m, such as from 3 m to 14 m, 3 m to 13 m, 3 m to 12 m, 3 m to 11 m, 3 m to 10 m, 4 m to 15 m, such as from 4 m to 14 m, 4 m to 13 m, 4 m to 12 m, 4 m to 11 m, 4 m to 10 m, 5 m to 15 m, such as from 5 m to 14 m, 5 m to 13 m, 5 m to 12 m, 5 m toll m, 5 m to 10 m, such as (about) 1, 2, 3, 4, 5, 6, 7, 8, 9, 10, 11, 12, 13, 14, or 15 m. In certain embodiments, each parallel row of the second plant or plant population, line, cultivar, or variety is at most 15 m long, preferably ranging from 1 m to 15 m, such as from 1 m to 14 m, 1 m to 13 m, 1 m to 12 m, 1 m to 11 m, 1 m to 10 m, 2 m to 15 m, such as from 12 m to 14 m, 2 m to 13 m, 2 m to 12 m, 2 m to 11 m, 2 m to 10 m, 3 m to 15 m, such as from 3 m to 14 m, 3 m to 13 m, 3 m to 12 m, 3 m to 11 m, 3 m to 10 m, 4 m to 15 m, such as from 4 m to 14 m, 4 m to 13 m, 4 m to 12 m, 4 m to 11 m, 4 m to 10 m, 5 m to 15 m, such as from 5 m to 14 m, 5 m to 13 m, 5 m to 12 m, 5 m to 11 m, 5 m to 10 m, such as (about) 1, 2, 3, 4, 5, 6, 7, 8, 9, 10, 11, 12, 13, 14, or 15 m.
C4 In certain embodiments, each row of the first and second plant or plant population, line, cultivar, or variety is at most 15 m long, preferably ranging from 1 m to 15 m, such as from 1 m to 14 m, 1 m to 13 m, 1 m to 12 m, 1 m to 11 m, 1 m to 10 m, 2 m to 15 m, such as from 12 m to 14 m, 2 m to 13 m, 2 m to 12 m, 2 m to 11 m, 2 m to 10 m, 3 m to 15 m, such as from 3 m to 14 m, 3 m to 13 m, 3 m to 12 m, 3 m to 11 m, 3 m to 10 m, 4 m to 15 m, such as from 4 m to 14 m, 4 m to 13 m, 4 m to 12 m, 4 m to 11 m, 4 m to 10 m, 5 m to 15 m, such as from 5 m to 14 m, 5 m to 13 m, 5 m to 12 m, 5 m to 11 m, 5 m to 10 m, such as (about) 1, 2, 3, 4, 5, 6, 7, 8, 9, 10, 11, 12, 13, 14, or 15 m. In certain embodiments, each parallel row of the first and second plant or plant population, line, cultivar, or variety is at most 15 m long, preferably ranging from 1 m to 15 m, such as from 1 m to 14 m, 1 m to 13 m, 1 m to 12 m, 1 m to 11 m, 1 m to 10 m, 2 m to 15 m, such as from 12 m to 14 m, 2 m to 13 m, 2 m to 12 m, 2 m to 11 m, 2 m to 10 m, 3 m to 15 m, such as from 3 m to 14 m, 3 m to 13 m, 3 m to 12 m, 3 m to 11 m, 3 m to 10 m, 4 m to 15 m, such as from 4 m to 14 m, 4 m to 13 m, 4 m to 12 m, 4 m to 11 m, 4 m to 10 m, 5 m to 15 m, such as from 5 m to 14 m, 5 m to 13 m, 5 m to 12 m, 5 m to 11 m, 5 m to 10 m, such as (about) 1, 2, 3, 4, 5, 6, 7, 8, 9, 10, 11, 12, 13, 14, or 15 m.

It will be understood that (parallel) rows within a specific plant or plant population, line, cultivar, or variety may have the same or a different length and that rows between different plants or plant populations, lines, cultivars, or varieties may have the same or a different length. By means of example and without limitations, a first row of plant line A may have a length of 15 m, a second row of plant line A may have a length of 10 m, and a row of plant line B may have a length of 12 m.

The skilled person will understand that typically a row is straight or about straight. However, curved rows are also within the scope of the present invention.

Aspect D
D1 In certain embodiments, the plants (or seeds) in each row are spaced apart from 1 to 50 cm, such as from 1 to 40 cm, 1 to 30 cm, 1 to 20 cm, 1 to 10 cm, 5 to 50 cm, 5 to 40 cm, 5 to 30 cm, 5 to 20 cm, 5 to 10 cm, 10 to 50 cm, 10 to 40 cm, 10 to 30 cm, 10 to 20 cm, 20 to 50 cm, 20 to 40 cm, 20 to 30 cm, 30 to 50 cm, 30 to 40 cm, or 40 to 50 cm. In certain embodiments, the plants (or seeds) in each parallel row are spaced apart from 1 to 50 cm, such as from 1 to 40 cm, 1 to 30 cm, 1 to 20 cm, 1 to 10 cm, 5 to 50 cm, 5 to 40 cm, 5 to 30 cm, 5 to 20 cm, 5 to 10 cm, 10 to 50 cm, 10 to 40 cm, 10 to 30 cm, 10 to 20 cm, 20 to 50 cm, 20 to 40 cm, 20 to 30 cm, 30 to 50 cm, 30 to 40 cm, or 40 to 50 cm.
D2 In certain embodiments, the plants (or seeds) in each row of the first plant or plant population, line, cultivar, or variety are spaced apart from 1 to 50 cm, such as from 1 to 40 cm, 1 to 30 cm, 1 to 20 cm, 1 to 10 cm, 5 to 50 cm, 5 to 40 cm, 5 to 30 cm, 5 to 20 cm, 5 to 10 cm, 10 to 50 cm, 10 to 40 cm, 10 to 30 cm, 10 to 20 cm, 20 to 50 cm, 20 to 40 cm, 20 to 30 cm, 30 to 50 cm, 30 to 40 cm, or 40 to 50 cm. In certain embodiments, the plants (or seeds) in each parallel row of the first plant or plant population, line, cultivar, or variety are spaced apart from 1 to 50 cm, such as from 1 to 40 cm, 1 to 30 cm, 1 to 20 cm, 1 to 10 cm, 5 to 50 cm, 5 to 40 cm, 5 to 30 cm, 5 to 20 cm, 5 to 10 cm, 10 to 50 cm, 10 to 40 cm, 10 to 30 cm, 10 to 20 cm, 20 to 50 cm, 20 to 40 cm, 20 to 30 cm, 30 to 50 cm, 30 to 40 cm, or 40 to 50 cm.
D3 In certain embodiments, the plants (or seeds) in each row of the second plant or plant population, line, cultivar, or variety are spaced apart from 1 to 50 cm, such as from 1 to 40 cm, 1 to 30 cm, 1 to 20 cm, 1 to 10 cm, 5 to 50 cm, 5 to 40 cm, 5 to 30 cm, 5 to 20 cm, 5 to 10 cm, 10 to 50 cm, 10 to 40 cm, 10 to 30 cm, 10 to 20 cm, 20 to 50 cm, 20 to 40 cm, 20 to 30 cm, 30 to 50 cm, 30 to 40 cm, or 40 to 50 cm. In certain embodiments, the plants (or seeds) in each parallel row of the second plant or plant population, line, cultivar, or variety are spaced apart from 1 to 50 cm, such as from 1 to 40 cm, 1 to 30 cm, 1 to 20 cm, 1 to 10 cm, 5 to 50 cm, 5 to 40 cm, 5 to 30 cm, 5 to 20 cm, 5 to 10 cm, 10 to 50 cm, 10 to 40 cm, 10 to 30 cm, 10 to 20 cm, 20 to 50 cm, 20 to 40 cm, 20 to 30 cm, 30 to 50 cm, 30 to 40 cm, or 40 to 50 cm.
D4 In certain embodiments, the plants (or seeds) in each row of the first and second plant or plant population, line, cultivar, or variety are spaced apart from 1 to 50 cm, such as from 1 to 40 cm, 1 to 30 cm, 1 to 20 cm, 1 to 10 cm, 5 to 50 cm, 5 to 40 cm, 5 to 30 cm, 5 to 20 cm, 5 to 10 cm, 10 to 50 cm, 10 to 40 cm, 10 to 30 cm, 10 to 20 cm, 20 to 50 cm, 20 to 40 cm, 20 to 30 cm, 30 to 50 cm, 30 to 40 cm, or 40 to 50 cm. In certain embodiments, the plants (or seeds) in each parallel row of the first and second plant or plant population, line, cultivar, or variety are spaced apart from 1 to 50 cm, such as from 1 to 40 cm, 1 to 30 cm, 1 to 20 cm, 1 to 10 cm, 5 to 50 cm, 5 to 40 cm, 5 to 30 cm, 5 to 20 cm, 5 to 10 cm, 10 to 50 cm, 10 to 40 cm, 10 to 30 cm, 10 to 20 cm, 20 to 50 cm, 20 to 40 cm, 20 to 30 cm, 30 to 50 cm, 30 to 40 cm, or 40 to 50 cm.

It will be understood that (parallel) rows within a specific plant or plant population, line, cultivar, or variety may have the same or a different (average or mean) spacing of plants within a row and that rows between different plants or plant populations, lines, cultivars, or varieties may have the same or a different (average or mean) spacing within a row. By means of example and without limitations, in a row of plant line A individual plants may be (on average) spaced apart 20 cm, in a different row of plant line A individual plants may be (on average) spaced apart 30 cm, and in a row of plant line B individual plants may be (on average) spaced apart 15 cm.

It will be understood that in the context of the present invention, the term “spaced apart” refers to the (average or mean) distance between plants (or seeds) within a row.

Aspect E
E1 In certain embodiments, the density of plants (in each row or area) ranges from 10 to 500 plants/m², such as from 10 to 400 plants/m², 10 to 300 plants/m², 10 to 200 plants/m², 10 to 100 plants/m², 50 to 500 plants/m², 50 to 400 plants/m², 50 to 300 plants/m², 50 to 200 plants/m², 50 to 100 plants/m², 100 to 500 plants/m², 100 to 400 plants/m², 100 to 300 plants/m², 100 to 200 plants/m², 200 to 500 plants/m², 200 to 400 plants/m², 200 to 300 plants/m², 300 to 500 plants/m², 300 to 400 plants/m², or 400 to 500 plants/m².
E2 In certain embodiments, the density of plants (in each row or area) of the first plant or plant population, line, cultivar, or variety ranges from 10 to 500 plants/m², such as from 10 to 400 plants/m², 10 to 300 plants/m², 10 to 200 plants/m², 10 to 100 plants/m², 50 to 500 plants/m², 50 to 400 plants/m², 50 to 300 plants/m², 50 to 200 plants/m², 50 to 100 plants/m², 100 to 500 plants/m², 100 to 400 plants/m², 100 to 300 plants/m², 100 to 200 plants/m², 200 to 500 plants/m², 200 to 400 plants/m², 200 to 300 plants/m², 300 to 500 plants/m², 300 to 400 plants/m², or 400 to 500 plants/m².
E3 In certain embodiments, the density of plants (in each row or area) of the second plant or plant population, line, cultivar, or variety ranges from 10 to 500 plants/m², such as from 10 to 400 plants/m², 10 to 300 plants/m², 10 to 200 plants/m², 10 to 100 plants/m², 50 to 500 plants/m², 50 to 400 plants/m², 50 to 300 plants/m², 50 to 200 plants/m², 50 to 100 plants/m², 100 to 500 plants/m², 100 to 400 plants/m², 100 to 300 plants/m², 100 to 200 plants/m², 200 to 500 plants/m², 200 to 400 plants/m², 200 to 300 plants/m², 300 to 500 plants/m², 300 to 400 plants/m², or 400 to 500 plants/m².
E4 In certain embodiments, the density of plants (in each row or area) of the first and second plant or plant population, line, cultivar, or variety ranges from 10 to 500 plants/m², such as from 10 to 400 plants/m², 10 to 300 plants/m², 10 to 200 plants/m², 10 to 100 plants/m², 50 to 500 plants/m², 50 to 400 plants/m², 50 to 300 plants/m², 50 to 200 plants/m², 50 to 100 plants/m², 100 to 500 plants/m², 100 to 400 plants/m², 100 to 300 plants/m², 100 to 200 plants/m², 200 to 500 plants/m², 200 to 400 plants/m², 200 to 300 plants/m², 300 to 500 plants/m², 300 to 400 plants/m², or 400 to 500 plants/m².

It will be understood that the density may vary locally and that preferably the average or mean density is calculated.

It will be understood that (parallel) rows or areas within a specific plant or plant population, line, cultivar, or variety may have the same or a different (average or mean) density of plants within a (parallel) row or area and that (parallel) rows or areas between different plants or plant populations, lines, cultivars, or varieties may have the same or a different (average or mean) spacing within a (parallel) row or area. By means of example and without limitations, in a row or area of plant line A plant density may be (on average) be 200 plants/m², in a different row or area of plant line A plant density may be (on average) 150 plants/m², and in a row or area of plant line B plant density may be (on average) 300 plants/m².

Aspect F
F1 In certain embodiments, the density of plants in each row ranges from 2 to 100 plants/m, such as from 5 to 100 plants/m, 10 to 100 plants/m, 20 to 100 plants/m, 40 to 100 plants/m, 60 to 100 plants/m, 80 to 100 plants/m, 5 to 80 plants/m, 10 to 80 plants/m, 20 to 80 plants/m, 40 to 80 plants/m, 60 to 80 plants/m, 5 to 60 plants/m, 10 to 60 plants/m, 20 to 60 plants/m, 40 to 60 plants/m, 5 to 40 plants/m, 10 to 40 plants/m, 20 to 40 plants/m, 5 to 20 plants/m, or 10 to 20 plants/m. In certain embodiments, the density of plants in each parallel row ranges from 2 to 100 plants/m, such as from 5 to 100 plants/m, 10 to 100 plants/m, 20 to 100 plants/m, 40 to 100 plants/m, 60 to 100 plants/m, 80 to 100 plants/m, 5 to 80 plants/m, 10 to 80 plants/m, 20 to 80 plants/m, 40 to 80 plants/m, 60 to 80 plants/m, to 60 plants/m, 10 to 60 plants/m, 20 to 60 plants/m, 40 to 60 plants/m, 5 to 40 plants/m, 10 to 40 plants/m, 20 to 40 plants/m, 5 to 20 plants/m, or 10 to 20 plants/m.
F2 In certain embodiments, the density of plants in each row of the first plant or plant population, line, cultivar, or variety ranges from 2 to 100 plants/m, such as from 5 to 100 plants/m, 10 to 100 plants/m, 20 to 100 plants/m, 40 to 100 plants/m, 60 to 100 plants/m, 80 to 100 plants/m, 5 to 80 plants/m, 10 to 80 plants/m, 20 to 80 plants/m, 40 to 80 plants/m, 60 to 80 plants/m, 5 to 60 plants/m, 10 to 60 plants/m, 20 to 60 plants/m, 40 to 60 plants/m, 5 to 40 plants/m, 10 to 40 plants/m, 20 to 40 plants/m, 5 to 20 plants/m, or 10 to 20 plants/m. In certain embodiments, the density of plants in each parallel row of the first plant or plant population, line, cultivar, or variety ranges from 2 to 100 plants/m, such as from 5 to 100 plants/m, 10 to 100 plants/m, 20 to 100 plants/m, 40 to 100 plants/m, 60 to 100 plants/m, 80 to 100 plants/m, 5 to 80 plants/m, 10 to 80 plants/m, 20 to 80 plants/m, 40 to 80 plants/m, 60 to 80 plants/m, 5 to 60 plants/m, 10 to 60 plants/m, 20 to 60 plants/m, 40 to 60 plants/m, 5 to 40 plants/m, 10 to 40 plants/m, 20 to 40 plants/m, 5 to 20 plants/m, or 10 to 20 plants/m.
F3 In certain embodiments, the density of plants in each row of the second plant or plant population, line, cultivar, or variety ranges from 2 to 100 plants/m, such as from 5 to 100 plants/m, 10 to 100 plants/m, 20 to 100 plants/m, 40 to 100 plants/m, 60 to 100 plants/m, 80 to 100 plants/m, 5 to 80 plants/m, 10 to 80 plants/m, 20 to 80 plants/m, 40 to 80 plants/m, 60 to 80 plants/m, 5 to 60 plants/m, 10 to 60 plants/m, 20 to 60 plants/m, 40 to 60 plants/m, 5 to 40 plants/m, 10 to 40 plants/m, 20 to 40 plants/m, 5 to 20 plants/m, or 10 to 20 plants/m. In certain embodiments, the density of plants in each parallel row of the second plant or plant population, line, cultivar, or variety ranges from 2 to 100 plants/m, such as from 5 to 100 plants/m, 10 to 100 plants/m, 20 to 100 plants/m, 40 to 100 plants/m, 60 to 100 plants/m, 80 to 100 plants/m, 5 to 80 plants/m, 10 to 80 plants/m, 20 to 80 plants/m, 40 to 80 plants/m, 60 to 80 plants/m, 5 to 60 plants/m, 10 to 60 plants/m, 20 to 60 plants/m, 40 to 60 plants/m, 5 to 40 plants/m, 10 to 40 plants/m, 20 to 40 plants/m, 5 to 20 plants/m, or 10 to 20 plants/m.
F4 In certain embodiments, the density of plants in each row of the first and second plant or plant population, line, cultivar, or variety ranges from 2 to 100 plants/m, such as from 5 to 100 plants/m, 10 to 100 plants/m, 20 to 100 plants/m, 40 to 100 plants/m, 60 to 100 plants/m, 80 to 100 plants/m, 5 to 80 plants/m, 10 to 80 plants/m, 20 to 80 plants/m, 40 to 80 plants/m, 60 to 80 plants/m, 5 to 60 plants/m, 10 to 60 plants/m, 20 to 60 plants/m, 40 to 60 plants/m, 5 to 40 plants/m, 10 to 40 plants/m, 20 to 40 plants/m, 5 to 20 plants/m, or 10 to 20 plants/m. In certain embodiments, the density of plants in each parallel row of the first and second plant or plant population, line, cultivar, or variety ranges from 2 to 100 plants/m, such as from 5 to 100 plants/m, 10 to 100 plants/m, 20 to 100 plants/m, 40 to 100 plants/m, 60 to 100 plants/m, 80 to 100 plants/m, 5 to 80 plants/m, 10 to 80 plants/m, 20 to 80 plants/m, 40 to 80 plants/m, 60 to 80 plants/m, 5 to 60 plants/m, 10 to 60 plants/m, 20 to 60 plants/m, 40 to 60 plants/m, 5 to 40 plants/m, 10 to 40 plants/m, 20 to 40 plants/m, 5 to 20 plants/m, or 10 to 20 plants/m.

It will be understood that the density may vary locally and that preferably the average or mean density is calculated.

It will be understood that (parallel) rows within a specific plant or plant population, line, cultivar, or variety may have the same or a different (average or mean) density of plants within a (parallel) row and that (parallel) rows or areas between different plants or plant populations, lines, cultivars, or varieties may have the same or a different (average or mean) spacing within a (parallel) row. By means of example and without limitations, in a row of plant line A plant density may be (on average) be 50 plants/m, in a different row of plant line A plant density may be (on average) 20 plants/m, and in a row of plant line B plant density may be (on average) 30 plants/m.

Aspect G
G1 In certain embodiments, the number of plants (in total or in each (parallel) row or area) ranges from 10 to 1000 plants, such as from 10 to 800 plants, 10 to 600 plants, 10 to 400 plants, 10 to 200 plants, 50 to 1000 plants, 50 to 800 plants, 50 to 600 plants, 50 to 600 plants, 50 to 400 plants, 50 to 200 plants, 100 to 1000 plants, 100 to 800 plants, 100 to 600 plants, 100 to 400 plants, 300 to 1000 plants, 300 to 800 plants, 300 to 600 plants, 500 to 1000 plants, 500 to 800 plants or 700 to 1000 plants.
G2 In certain embodiments, the number of plants (in total or in each (parallel) row or area) of the first plant or plant population, line, cultivar, or variety ranges from 10 to 1000 plants, such as from 10 to 800 plants, 10 to 600 plants, 10 to 400 plants, 10 to 200 plants, 50 to 1000 plants, 50 to 800 plants, 50 to 600 plants, 50 to 600 plants, 50 to 400 plants, 50 to 200 plants, 100 to 1000 plants, 100 to 800 plants, 100 to 600 plants, 100 to 400 plants, 300 to 1000 plants, 300 to 800 plants, 300 to 600 plants, 500 to 1000 plants, 500 to 800 plants or 700 to 1000 plants.
G3 In certain embodiments, the number of plants (in total or in each (parallel) row or area) of the second plant or plant population, line, cultivar, or variety ranges from 10 to 1000 plants, such as from 10 to 800 plants, 10 to 600 plants, 10 to 400 plants, 10 to 200 plants, 50 to 1000 plants, 50 to 800 plants, 50 to 600 plants, 50 to 600 plants, 50 to 400 plants, 50 to 200 plants, 100 to 1000 plants, 100 to 800 plants, 100 to 600 plants, 100 to 400 plants, 300 to 1000 plants, 300 to 800 plants, 300 to 600 plants, 500 to 1000 plants, 500 to 800 plants or 700 to 1000 plants.
G4 In certain embodiments, the number of plants (in total or in each (parallel) row or area) of the first and second plant or plant population, line, cultivar, or variety ranges from 10 to 1000 plants, such as from 10 to 800 plants, 10 to 600 plants, 10 to 400 plants, 10 to 200 plants, 50 to 1000 plants, 50 to 800 plants, 50 to 600 plants, 50 to 600 plants, 50 to 400 plants, 50 to 200 plants, 100 to 1000 plants, 100 to 800 plants, 100 to 600 plants, 100 to 400 plants, 300 to 1000 plants, 300 to 800 plants, 300 to 600 plants, 500 to 1000 plants, 500 to 800 plants or 700 to 1000 plants.

It will be understood that (parallel) rows or areas within a specific plant or plant population, line, cultivar, or variety may have the same or a different number of plants within a (parallel) row or area and that (parallel) rows or areas between different plants or plant populations, lines, cultivars, or varieties may have the same or a different number of plants within a (parallel) row or are. By means of example and without limitations, in a row or area of plant line A plant number may be 500 plants, in a different row or area of plant line A plant number may be 300 plants, and in a row or area of plant line B plant number may be 800 plants.

Combination of Plant Scheme Features

In certain embodiments, plants are planted, or seeds thereof are sown or have been sown in one or more (parallel) rows, preferably wherein
a) the number of rows of a first and/or second plant or plant population, line, cultivar, or variety ranges from 2 to 10, preferably 2 to 5, more preferably 2 to 3; and/or
b) each row of a first and/or second plant or plant population, line, cultivar, or variety is at most 1 m spaced apart, preferably ranging from 10 cm to 1 m, such as from 10 cm to 90 cm, 10 cm to 80 cm, 10 cm to 70 cm, 10 cm to 50 cm, 10 cm to 40 cm 10 cm to 30 cm, or 10 cm to 20 cm; and/or
c) each row of a first and/or second plant or plant population, line, cultivar, or variety is at most 15 m long, preferably ranging from 1 m to 15 m, such as from 1 m to 10 m, 1 m to 5 m, 2 m to 15 m, 2 m to 10 m, 2 m to 5 m, 5 m to 15 m, or 5 m to 10 m; and/or
d) the plants (or seeds) in each row of a first and/or second plant or plant population, line, cultivar, or variety are spaced apart from 1 to 50 cm, such as from 1 cm to 40 cm, 1 cm to 30 cm, 1 cm to 20 cm, 1 cm to 10 cm, 5 cm to 50 cm, 5 cm to 40 cm, 5 cm to 30 cm, 5 cm to 20 cm, 10 cm to 50 cm, 10 cm to 40 cm, 10 cm to 30 cm, or 10 cm to 20 cm; and/or
e) the density of plants in each row or area of a first and/or second plant or plant, line, cultivar, or variety population ranges from 10 to 500 plants/m², such as from 100 to 500 plants/m², 200 to 500 plants/m², 300 to 500 plants/m², or 400 to 500 plants/m²; and/or
f) the density of plants in each row of a first and/or second plant or plant population, line, cultivar, or variety ranges from 2 to 100 plants/m, preferably 5 to 100 plants/m, such as 10 to 100 plants/m, 30 to 100 plants/m, 50 to 100 plants/m, or 70 to 100 plants/m; and/or
g) the number of plants (in total or in each (parallel) row or area of a first and/or second plant or plant population, line, cultivar, or variety) ranges from 10 to 1000 plants, preferably 50 to 1000, such as 100 to 1000, 300 to 1000, 500 to 1000, or 700 to 1000.

In certain embodiments, planting schemes combine any one or more of features {a}, {b}, {c}, {d}, {e}, {f}, {g}, {a+b}, {a+c}, {a+d}, {a+e}, {a+f}, {a+g}, {b+c}, {b+d}, {b+e}, {b+f}, {b+g}, {c+d}, {c+e}, {c+f}, {c+g}, {d+e}, {d+f}, {d+g}, {e+f}, {e+g}, {f+g}, {a+b+c}, {a+b+d}, {a+b+e}, {a+b+f}, {a+b+g}, {a+c+d}, {a+c+e}, {a+c+f}, {a+c+g}, {a+d+e}, {a+d+f}, {a+d+g}, {a+e+f}, {a+e+g}, {a+f+g}, {b+c+d}, {b+c+e}, {b+c+f}, {b+c+g}, {b+d+e}, {b+d+f}, {b+d+g}, {b+e+f}, {b+e+g}, {b+f+g}, {c+d+e}, {c+d+f}, {c+d+g}, {c+e+f}, {c+e+g}, {c+f+g}, {d+e+f}, {d+e+g}, {d+f+g}, {e+f+g}, {a+b+c+d}, {a+b+c+e}, {a+b+c+f}, {a+b+c+g}, {a+b+d+e}, {a+b+d+f}, {a+b+d+g}, {a+b+e+f}, {a+b+e+g}, {a+b+f+g}, {a+c+d+e}, {a+c+d+f}, {a+c+d+g}, {a+c+e+f}, {a+c+e+g}, {a+c+f+g}, {a+d+e+f}, {a+d+e+g}, {a+d+f+g}, {a+e+f+g}, {b+c+d+e}, {b+c+d+f}, {b+c+d+g}, {b+c+e+f}, {b+c+e+g}, {b+c+f+g}, {b+d+e+f}, {b+d+e+g}, {b+d+f+g}, {b+e+f+g}, {c+d+e+f}, {c+d+e+g}, {c+d+f+g}, {c+e+f+g}, {d+e+f+g}, {a+b+c+d+e}, {a+b+c+d+f}, {a+b+c+d+g}, {a+b+c+e+f}, {a+b+c+e+g}, {a+b+c+f+g}, {a+b+d+e+f}, {a+b+d+e+g}, {a+b+d+f+g}, {a+b+e+f+g}, {a+c+d+e+f}, {a+c+d+e+g}, {a+c+d+f+g}, {a+c+e+f+g}, {a+d+e+f+g}, {b+c+d+e+f}, {b+c+d+e+g}, {b+c+d+f+g}, {b+c+e+f+g}, {b+d+e+f+g}, {c+d+e+f+g}, {a+b+c+d+e+f}, {a+b+c+d+e+g}, {a+b+c+d+f+g}, {a+b+c+e+f+g}, {a+b+d+e+f+g}, {a+c+d+e+f+g}, {b+c+d+e+f+g}, {a+b+c+d+e+f+g}.

The aspects and embodiments of the invention are further supported by the following non-limiting examples. The following examples, including the experiments conducted and the results achieved, are provided for illustrative purposes only and are not constructed as limiting the present invention.

### EXAMPLES

### Example 1: Hybrid Seed Production

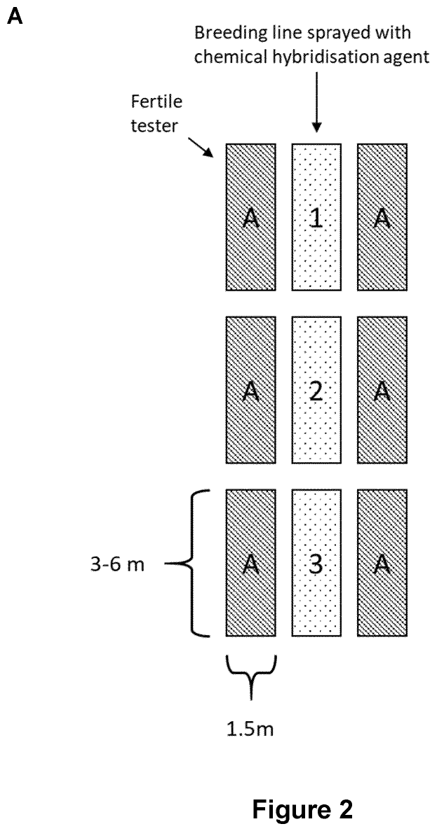

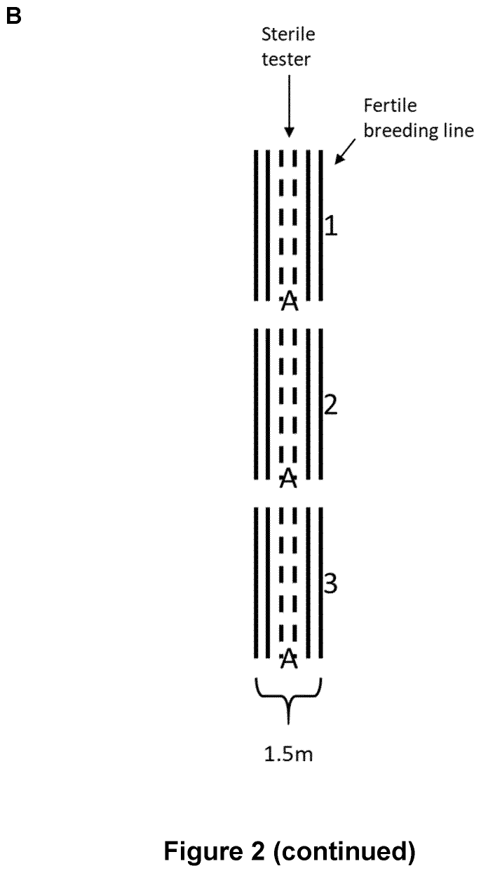

697 unique spring wheat hybrids were produced using the following procedure. The lines were drilled using a 6-row Hege-drill, drilling 6 m rows with two male sterile female rows in the center and two male rows on either side (see FIG. 2B).

The male lines were a mix of KWS breeding lines, Australian breeding lines and a wide collection of spring wheat germplasm obtained through various germplasm exchanges over the years.

The male sterile female lines were KWS breeding lines and Australian breeding lines all converted to be homozygous for genetic deletion on the region on chromosome 4B where the Ms1 gene is located.

The hybrids were based on parental lines from various countries globally, hence had a wide spread of anther extrusion (measured on 0-3 scale with 0=no anthers fully extruded and 3=all anthers fully extruded), as well as a wide range of anthesis dates with positive numbers showing the number of days male lines flowered after female lines, and negative numbers showing the number of days the males flowered before the female.

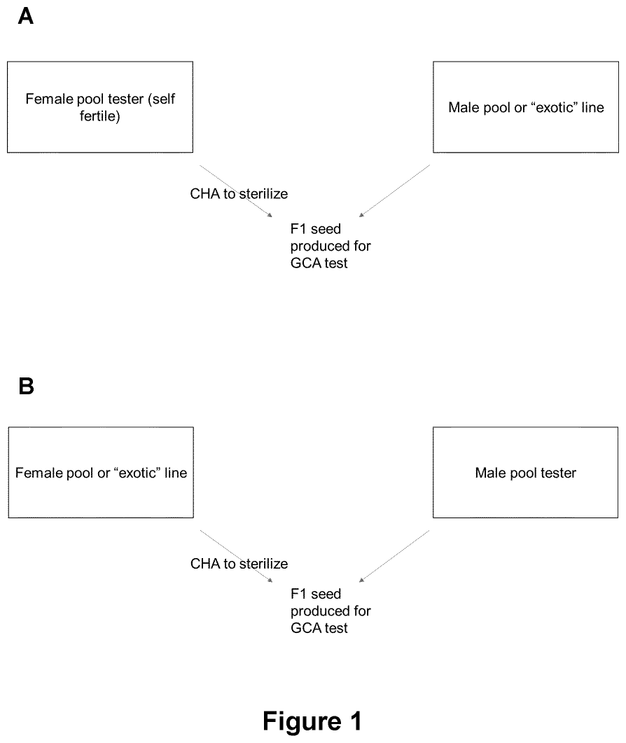

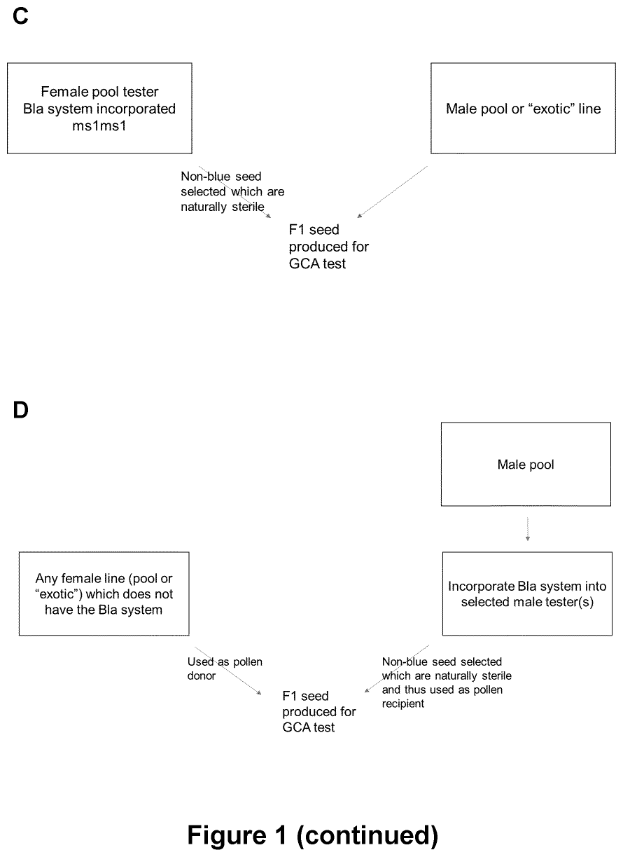

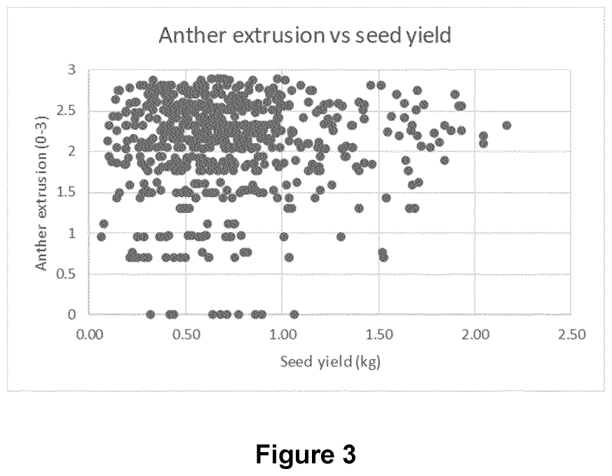

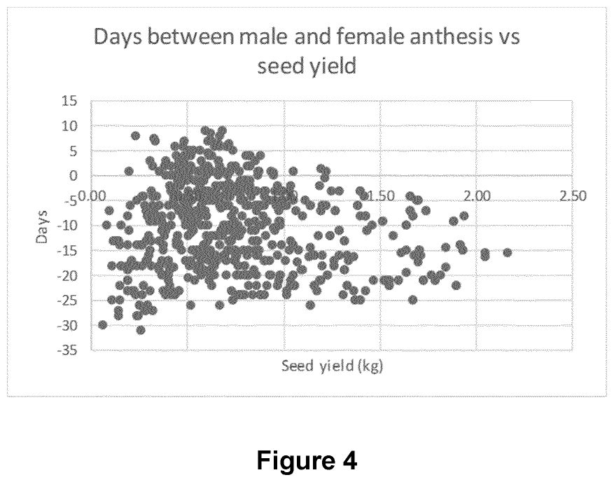

FIGS. 3 and 4 show the seed set in kilogram harvested from 6 m of double rows of females in relation to anther extrusion of the male used and difference in heading date between the male and female. FIG. 3 particularly exhibits that even the anther extrusion at lower level (0.5-1) can results in high level of yield. Also FIG. 4 presents that even when male and female are flowering at different days (period) a satisfied yield can be achieved. As is clear, there is no correlation between any of these traits. This can be explained by the abundance of males over females (ratio 2:1), the close proximity and the drilling with row spacing which encourage tillering and hence prolonging the time in which both males and female plants have activity flowering florets. This can advantageously be achieved by the use of a breeding scheme as for instance set forth in FIG. 2B and the use of male sterile testers (which can be selected from either the male pool or the female pool, such as for instance exemplified in FIGS. 1C and 1D).

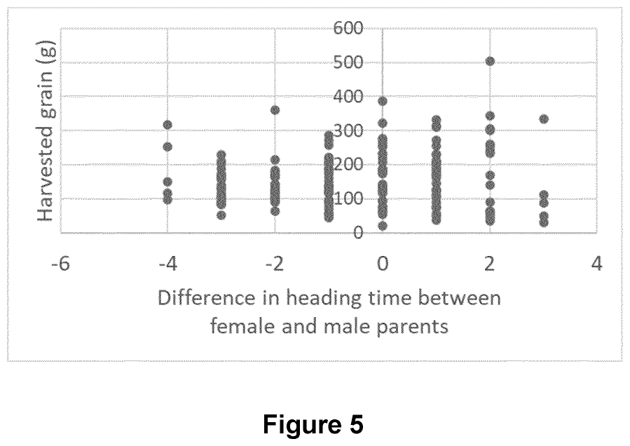

FIG. 5 shows the amount of harvested grain (in grams) in relation to the difference in heading time (in days) between female and male parents. Good seed production is obtained even when flowering was not completely synchronized.

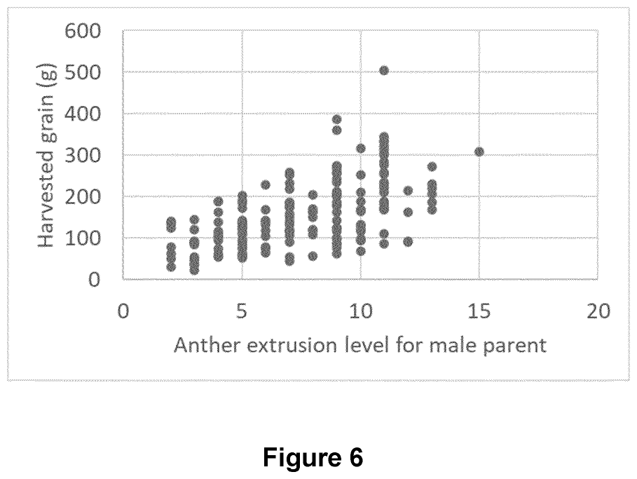

FIG. 6 shows the amount of harvested grain (in grams) in relation to the anther extrusion level for the male parent.

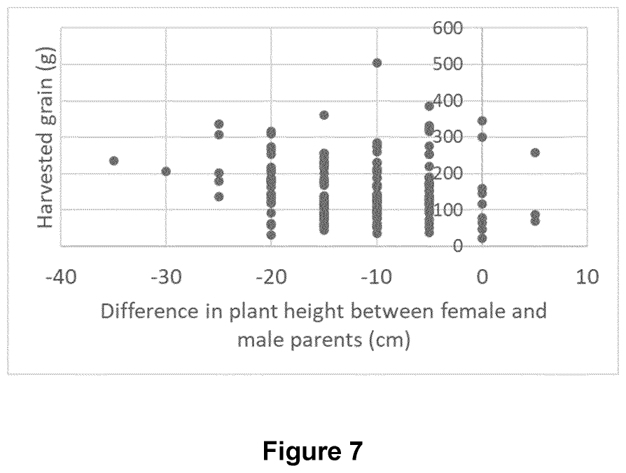

FIG. 7 shows the amount of harvested grain (in grams) in relation to the difference in plant height (in cm) between the female and male parent.

The results of FIGS. 5 to 7 show that hybrid test seed can be generated from combinations in which male and female plants differ in heading date, anther extrusion and plant height. While good anther extrusion provides higher seed set, even very poor anther extrusion leads to the generation of seed. These results further indicate that even with large (>2 days) differences in flowering time and with low anther extrusion it is still possible to produce useful amount of F1 seed for testing. In conclusion, good seed production is observed even when flowering was not completely synchronized.

1. A method for evaluating plant hybrid test crosses or for evaluating general and/or specific combining ability or heterosis in plant hybrid test crosses of or in a particular plant or plant population, line, cultivar, or variety or combination of parent plants of the genus Triticum, preferably Triticum aestivum, comprising
providing a F1 hybrid plant or part thereof, or plant population obtained by crossing said particular plant as a first or plant population, line, cultivar, or variety with a different plant or plant population, line, cultivar, or variety as a second plant or plant population, line, cultivar, or variety, wherein said first or second plant is a genetically male sterile plant obtained by selecting non-blue seed derived from a mix of seed collected from self-fertilised ms1-deletion plant having an alien addition chromosome comprising a restorer gene and BLA gene and wherein said other plant is a genetically male fertile plant, or wherein said first or second plant is a ms1-deletion plant having an alien addition chromosome comprising a restorer gene and BLA gene and wherein said other plant is a genetically male sterile plant;
determining one or more agronomic, physiologic, or quality characteristics or traits of said hybrid plant or plant population so as to evaluate the plant hybrid test cross or to determine the general and/or specific combining ability or heterosis of said particular plant or combination of parent plants.

2. The method according to claim 1, wherein said hybrid plant is obtained from a cross in which seeds of one or more first plant or plant population, line, cultivar, or variety or planting plants of one or more first plant or plant population, line, cultivar, or variety have been sown in one or more parallel row; and in which seeds of one or more second plant or plant population, line, cultivar, or variety or planting plants of one or more second plant or plant population, line, cultivar, or variety in one or more parallel row flanking or flanked by said one or more parallel row of said one or more first plant or plant population, line, cultivar, or variety have been sown.

3. A method for sowing or planting for generating a hybrid plant, evaluating heterosis or general/specific combining ability and/or for plant hybrid test crossing, comprising
sowing seeds of one or more first plant or plant population, line, cultivar, or variety of the genus Triticum, preferably Triticum aestivum, or planting plants of one or more first plant or plant population, line, cultivar, or variety of the genus Triticum, preferably Triticum aestivum in one or more parallel row;
sowing seeds of one or more second plant or plant population, line, cultivar, or variety of the genus Triticum, preferably Triticum aestivum, or planting plants of one or more second plant or plant population, line, cultivar, or variety of the genus Triticum, preferably Triticum aestivum in one or more parallel row flanking or flanked by said one or more parallel row of said one or more first plant or plant population, line, cultivar, or variety;
wherein said first or second plant or plant population, line, cultivar, or variety is a genetically male sterile plant or plant population, line, cultivar, or variety is obtained by selecting non-blue seed derived from a mix of seed collected from self-fertilised ms1-deletion plant having an alien addition chromosome comprising a restorer gene and BLA gene, or wherein said first or second plant is a ms1-deletion plant having an alien addition chromosome comprising a restorer gene and BLA gene and wherein said other plant is a genetically male sterile plant.

4. The method according to claim 2, wherein said one or more row of said one or more first and/or second plant or plant population, line, cultivar, or variety is at most 5 rows, preferably at most 4 rows, more preferably at most three rows, most preferably at most two rows.

5. The method according to claim 2, wherein each of said rows is at most 1 meter spaced apart.

6. The method according to any claim 2, wherein each of said rows is at most 15 m long.

7. The method according to claim 2, wherein individual plants within a row are spaced apart 1 to 50 cm.

8. The method according to claim 2, wherein the plant density of said first and/or second plant or plant population, line, cultivar, or variety is from 10 to 500 plants/m².

9. The method according to claim 2, wherein the number of individual plants of said one or more first and/or second plant or plant population, line, cultivar, or variety is from 10 to 1000.

10. The method according to claim 1, wherein said first or second plant or plant population, line, cultivar, or variety is a tester.

11. The method according to claim 1, wherein said first plant or plant population, line, cultivar, or variety is a male sterile plant or plant population, line, cultivar, or variety selected from plants or plant populations, lines, cultivars, or varieties in a male pool of plants or plant populations, lines, cultivars, or varieties.

12. The method according to claim 11, wherein said second plant or plant population, line, cultivar, or variety is selected from plants or plant populations, lines, cultivars, or varieties in a female pool of plants or plant populations, lines, cultivars, or varieties.

13. The method according to any claim 1, wherein said method does not involve the use of a chemical hybridization agent and/or the use of cytoplasmic male sterility.

14. The method according to n claim 1, wherein said genetically male sterile plant or plant population, line, cultivar, or variety comprises a mutation is the ms1 and/or ms5 gene, preferably a knockout mutation or a frameshift mutation.

15. Use of a genetically male sterile plant or plant population, line, cultivar, or variety or the genus Triticum, preferably Triticum aestivum, for evaluating heterosis or general/specific combining ability, or for plant hybrid test crossing, preferably wherein said plant or plant population, line, cultivar, or variety is planted or sown as defined in claim 2.
